(19) United States
(12) Patent Application Publication
(10) Pub. No.: US 2019/0015518 A1
(43) Pub. Date: Jan. 17, 2019

(54) DRUG DELIVERY COMPOSITIONS AND METHODS

(71) Applicants: UNIVERSITY OF KANSAS, LAWRENCE, KS (US); NANOPHARM LLC D/B/A HYLAPHARM, LAWRENCE, KS (US)

(72) Inventors: MARCUS LAIRD FORREST, LAWRENCE, KS (US); SHUANG CAI, LAWRENCE, KS (US); TI ZHANG, LAWRENCE, KS (US); SP SANJEEWA NILENDRA SENADHEERA, LAWRENCE, KS (US); CHAD GROER, LAWRENCE, KS (US); DANIEL AIRES, MISSION HILLS, KS (US)

(21) Appl. No.: 16/067,630

(22) PCT Filed: Jan. 4, 2017
(86) PCT No.: PCT/US2017/012134

§ 371 Date: Jul. 2, 2018

Related U.S. Application Data
(60) Provisional application No. 62/274,508, filed on Jan. 4, 2016.

(51) Int. Cl. A61K 47/61; A61K 33/24; A61K 31/282; A61K 9/19

(52) U.S. Cl. CPC A61K 47/61; A61K 9/19; A61K 31/282; A61K 33/24

(57) ABSTRACT

One aspect of the present invention is directed to conjugates that comprise a linear polymer carrier, a linker and one or more drugs or imaging agents, as well as pharmaceutical compositions that include such conjugates. The drug may be a platinum-containing drug with a linker comprised of a modified amino acid. The conjugate may alternatively include a metal complexing ligand with a metal used for imaging or chemotherapeutic purposes. Another aspect of the invention is directed to formulations and processes for lyophilization of hyaluronan conjugates. Another aspect of the invention is directed to methods for treating and/or inhibiting cancer utilizing the conjugates and compositions described herein.

### CROSS-REFERENCE TO RELATED APPLICATIONS

This application is based on and claims priority to U.S. Provisional Application Ser. No. 62/274,508 filed on Jan. 4, 2016, which is hereby incorporated herein by reference.

### BACKGROUND OF THE INVENTION

Over one thousand cisplatin analogues have been developed since the 1960s to improve tolerability, broaden activity, and to overcome drug resistance. Yet, less than 20 candidates have made it to different phases of human clinical trials, and only 3 have regulatory approvals in the US. They are the original cisplatin initially approved for ovarian and testicular cancers in 1978, carboplatin initially approved in 1988 for palliative treatment of recurrent ovarian cancer, and oxaliplatin initially approved in 2002 for second line treatment of metastatic colorectal cancer. Cisplatin remains the most potent in most indications, but these analogues have equivalent overall survival and/or remission rates and reduced toxicity in the approved indications.

Despite different chemical structures, cisplatin and its analogues undergo similar hydrolysis reactions in vivo and form the active platinum species cis-[Pt(X)₂(OH₂)(Y)]⁺, (Pt-monoaqua) and cis-[Pt(X)₂(OH₂)₂]²+, (Pt-diaqua). The aqua ligands on the Pt-monoaqua and Pt-diaqua are easily displaced by DNA bases, preferentially adenine and guanine, forming [Pt(X)₂(Y)(adenine-DNA)]⁺ and [Pt(X)₂(Y)(guanine-DNA)]⁺, subsequently [Pt(X)₂(adenine-DNA)₂]²⁻ and [Pt(Y)₂(guanine-DNA)₂]²⁺ via the displacement of the other ligands. Thus, the aquated platinum species lead to the formation of intra- and inter-strand DNA crosslinks in cancer cells, which interfere with cell division and DNA repair, and in turn trigger cell death. The ligands function primarily to alter the reactivity to the DNA bases and lipophilicity, and hence alter tissue distribution and pharmacokinetics.

Non-targeted platinum chemotherapy is toxic not only to cancer cells, but it also damages normal cells that divide rapidly. Approximately 30% of patients who received 50-100 mg/m² cisplatin as an initial dose developed severe nephrotoxicity, due to the damaged pars recta of the proximal tubules of the kidney, and over half of patients develop hypomagnesaemia and hypocalcaemia. In contrast, nephrotoxicity is greatly reduced in the carboplatin regimen, but thrombocytopenia, which is uncommon for cisplatin, is dose limiting in 25% of carboplatin patients.

The distinct toxicity profiles of cisplatin and carboplatin are likely attributed to the differences in the chemical reactivity, pharmacology, and toxicology of the two platinum compounds. The cyclobutane-dicarboxylate ligands of carboplatin are slowly hydrolyzed to form the same membrane impregnable aquated species as cisplatin, but the hydrolysis rate of carboplatin is approximately 10-fold slower than cisplatin. In terms of excretion, 25% of cisplatin is excreted unchanged in the urine, while 90% of carboplatin is unchanged. In regards to metabolism, cisplatin undergoes inactivation by sulfhydryl groups in vivo, while carboplatin is not significantly metabolized. These differences in pharmacokinetics may contribute to the longer elimination half-life and improved toxicity profile of carboplatin.

U.S. Pat. No 8,088,412, incorporated herein by reference, discloses a first-generation cisplatin-based polymeric conjugate (HA-Pt), using the biocompatible and non-immunogenic polysaccharide hyaluronan (HA) for locoregional delivery of platinum chemotherapy to treat spontaneous canine cancers. The conjugate was based on a direct attachment of cisplatin to the polymer, and it released active aquated cisplatin over several hours (t_{1/2} 10 hrs). It demonstrated superior pharmacokinetics in rodents and canines compared to cisplatin, and showed a 23% cure in heterogeneous oral squamous cell carcinomas in dogs (compared to 7% for cisplatin). The conjugate did not demonstrate renal toxicity in either rodents or dogs, which is the dose-limiting side effect of cisplatin chemotherapy. However, it resulted in myelosuppression, hepatic, and cardiac toxicities in some patients in a Phase I/II canine clinical trial. Platinum chemotherapeutics rarely cause hepatic and cardiac toxicities.

### BRIEF SUMMARY OF THE INVENTION

One aspect of the present invention is directed to a composition comprising a drug conjugate, where the drug conjugate comprises a hyaluronan, a chelated platinum, and a linker joining the hyaluronan and the platinum.

In certain embodiments the linker comprises an amine, amide or carboxyl group capable of chelating the platinum. In certain embodiments, the linker may comprise at least two groups selected from the group consisting of amine, amide and carboxyl, wherein each of the two groups may be the same or different. In certain embodiment, the linker comprises a modified amino acid capable of chelating the platinum, and in some such embodiments comprises one or two modified amino acids. In some such embodiments the linker comprises a derivative of lysine, which may be N-acetyl lysine. In certain embodiments, the linker comprises a ligand selected from the group consisting of diglycine, dicarboxylato, maloyl, and diamino. In certain embodiments, the linker comprises HOOC—X—NH—Y, wherein X is selected from the group consisting of CH₂, CH₂Z, and CHZCH₂, wherein Z is an aliphatic group, and wherein Y is a moiety selected from the group consisting of acetyl, ethyl and an aliphatic chain. In certain embodiments, the linker further comprises a hydrazide.

In certain embodiments, the platinum is in the II oxidative state. In some embodiments, the linker and the platinum form a ring selected from the group consisting of 5 member rings and 6 member rings. In certain embodiments, the platinum forms part of a drug selected from the group consisting of cisplatin, carboplatin and oxaliplatin.

In certain embodiments, the hyaluronan of the drug conjugate has a molecular weight from 6 to 300 kDa. In some embodiments, the composition further comprises, in addition to the drug conjugate, a second hyaluronan having a molecular weight greater than 600 kDa. In certain embodiments, the composition further comprises an excipient selected from the group consisting of a sugar, a monovalent or polyvalent cation, and combinations thereof. In some such embodiments, the sugar is mannitol, sucrose, or trehalose. In some such embodiments, the cation is a calcium salt or sodium salt.

Another aspect of the invention is directed to a method of treating and/or inhibiting cancer, the method including administering the composition of any of any of the aforementioned embodiments to a patient in need thereof.

Yet another aspect of the invention is directed to processes for lyophilizing a hyaluronan-drug conjugate. Such processes include forming a lyophilization formulation comprising a hyaluronan-drug conjugate and an excipient selected from the group consisting of a salt having a concentration from 0.01 to 0.9 wt %, a sugar having a concentration between 1 and 20 wt % and combinations thereof. In certain embodiments the salt is selected from the group consisting of calcium and sodium salts, and the sugar is selected from the group consisting of mannitol, sucrose, or trehalose.

In certain embodiments, the process further includes lyophilizing the conjugate. In certain such embodiments, the lyophilizing step comprises a freezing step following by a multistep drying process.

In certain embodiments the process further includes rehydrating the conjugate. In certain such embodiments the rehydrating step comprising rehydrating with a diluent comprising a hyaluronan with a molecular weight greater than 600 kDa.

In any of the aforementioned embodiments of the lyophilization processes, the hyaluronan-drug conjugate may be a hyaluronan-platinum conjugate. In some such embodiments the hyaluronan-platinum conjugate is any of the hyaluronan-platinum conjugates described with respect to the first aspect of the invention.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 includes a library of potential linkers.

FIG. 2 includes a drawing of the synthesis scheme of HA-Lys-Pt using 35- or 75-kDa HA.

FIG. 3 includes the synthesis of HA_{6.4}-cis-[Pt(NH₃)₂(N_{α}-Acetyl-L-lysine)] (HA_{6.4}-lys-Pt) conjugates.

FIG. 4A includes the Transmission Electron Microscopy (TEM) images of freshly made HA-Lys-Pt. The images were collected on a FEI Tecnai F20 XT Field Emission TEM. The diameters of HA-Lys-Pt nanoparticles ranged from 25-50 nm.

FIG. 4B includes the Transmission Electron Microscopy (TEM) images of freshly made HA-Lys-Pt after salt treatment for 2 weeks. The images were collected on a FEI Tecnai F20 XT Field Emission TEM. The diameters of HA-Lys-Pt nanoparticles ranged from 25-50 nm. Salt treated HA-Lys-Pt showed low intensity of Pt due to partial release of the platinum.

FIG. 5 includes the UV spectra of HA, cisplatin, HylaPlat (HA-Pt nanoparticles with direct Pt conjugation to the carboxylate of HA, and HA-Lys-Pt (75 kDa HA).

FIG. 6 includes a graph of the in vitro release of Pt(II) from HA-Lys-Pt in PBS at pH 7.4 or acetate at pH 5.5 and 37° C. Release half-lives were 49, 15, and 11 hours for 75, 35, and 6.4-kDa conjugates at pH 7.4, respectively, and 24 hours for the 75-kDa at pH 5.5.

FIG. 7 includes a graph of the degradation profiles of HA, HA-Lys, and 75HA-Lys-Pt by HAdase in 24 hours.

FIG. 8 includes a graph of the chromatograms of Pt species mixture solution and the filtrate of HA-Lys-Pt solution generated by cation exchange HPLC.

FIG. 9 includes the anti-proliferative activity graphs of HA-Lys-Pt conjugates in MDA-1986 human head and neck squamous cell carcinoma cell line. Cells were treated with increasing concentrations of compound for 72 hours and cell proliferation was quantified using Resazurin Blue. Data from at least three separate experiments performed in duplicate was analyzed by non-linear regression. Representative curves are shown. Complete data set is in Table 1.

FIG. 10 includes two graphs of the levels of platinum in A) Pt-DNA adducts and B) rat plasma. Rats were treated with cisplatin (i.v., 10 mg/kg) or HA-Lys-Pt (s.c.; 10 mg/kg cisplatin basis). Pt content was quantified by ICP-MS in white blood cell DNA and plasma. A) DNA adduct formation (mean±S.E.M.; n=2-6). Cisplatin Cₘₐₓ=16.1±5.3 pg Pt/μg DNA; Tₘₐₓ=1 hr; AUC=1255 pg Pt×h/μg DNA. HA-Lys-Pt Cₘₐₓ=135.1±45.2 pg Pt/μg DNA; Tₘₐₓ=48 hr; AUC=5247 pg Pt×h/μg DNA. B) Plasma Pt (mean±S.E.M.; n=2-6). Cisplatin Cₘₐₓ=7.0±2.1 μg Pt/mL plasma; Tₘₐₓ=0.25 hr; AUC=70.5 pg Pt×h/mL. HA-Lys-Pt Cₘₐₓ=4.1±2.4 μg Pt/mL plasma; Tₘₐₓ=24 hr; AUC=274.7 pg Pt×h/mL.

FIG. 11 includes two graphs of body weight. A) Percent weight loss after a single injection of 75HA-Lys-Pt at 20 (N=2), 30 (N=3), and 40 mg/kg (N=2). B) Percent weight loss after a single injection of 33HA-Lys-Pt at 8.86 (N=2), 13.3 (N=2), and 17.7 (N=1) mg/kg.

FIG. 12 includes four graphs. A) Growth of head and neck tumors after three weekly injections of saline (i.v., N=9), 75HA-Lys-Pt (20 mg/kg s.c., N=8), and cisplatin (10 mg/kg i.v., N=8); B) Tumor growth in individual mouse that received three weekly injections of 20 mg/kg 75HA-Lys-Pt subcutaneously; C) Percent weight loss of mice that received saline, 75HA-Lys-Pt, or cisplatin; D) Survival rate of the animals.

FIG. 13A includes a graph of the pharmacokinetics of HA-Lys-Pt, a four-compartmental model for analyzing the pharmacokinetics of the 75HA-Lys-Pt after sub-mucosal injection.

FIG. 13B includes a graph of the pharmacokinetics of HA-Lys-Pt, the serum platinum concentration vs. time curve for the first dose (1.5 mg/kg, sub-mucosal injection).

FIG. 13C includes a graph of the pharmacokinetics of HA-Lys-Pt, urine platinum concentration vs. time curve for the first dose (1.5 mg/kg, sub-mucosal injection).

FIG. 13D includes a graph of the pharmacokinetics of HA-Lys-Pt, the serum platinum concentration vs. time curve for the second dose (2.0 mg/kg, sub-mucosal injection). The urine platinum concentration vs. time curve for the second dose was not available due to the lack of urine samples for analysis and modeling.

FIGS. 14A, 14B, and 14C include three graphs of plasma cisplatin concentration vs. time curves. All three dogs were treated with an injection of 10 mg/m² 75HA-Lys-Pt. The pharmacokinetics were modeled using a SAAM II software.

FIG. 15 includes the synthetic scheme of HA-GluH-Pt.

FIG. 16 includes the release of HA-GluH-Pt. The release half-lives of HA-GluH-Pt were less than 2 hours in both PBS and acetate buffers.

FIG. 17 includes the HA-GluH-Pt degradation curve in the presence of hyaluronidase.

FIG. 18 includes cellular uptake result of HA-GluH-Pt in the presence of anti-CD44 antibody and chlorpromazine in MDA-1986 cells.

FIG. 19 includes the tumor size measurements after treatment of HA-GluH-Pt. HA-GluH-Pt was administered subcutaneously next to the tumor at a dose of 20 mg/kg on the basis of cisplatin. The HA-GluH-Pt was given weekly for 5 weeks.

FIGS. 20A, 20B, and 20C include three graphs of the viscosity of HA, HA-Lys, and HA-Lys-Pt in the presence of 0, 0.1, 0.5 and 1 M mannitol.

FIGS. 21A, 21B, and 21C include three graphs of the viscosity of HA, HA-Lys, and HA-Lys-Pt in the presence of 0, 0.1, 0.5 and 1 M sucrose.

FIGS. 22A, 22B, and 22C include three graphs of the viscosity of HA, HA-Lys, and HA-Lys-Pt in the presence of 0, 0.1, 0.5 and 1 M trehalose.

FIGS. 23A and 23B include two graphs of the viscosity of 75 kDa HA in the presence of 0, 12 and 24 mM CaCl₂ at pH 7 and 6.

FIGS. 24A and 24B include two graphs of the viscosity of 75HA-Lys-Pt in the presence of 0, 12 and 24 mM CaCl₂ at pH 5 and 6.

FIG. 25 includes the viscosity of 75HA-Lys-Pt in the presence of calcium acetate at pHs 5 and 6.

FIG. 26 includes the viscosity of 75HA-Lys-Pt in the presence of calcium lactate at pHs 5 and 6.

FIG. 27 includes the viscosity of 75HA-Lys-Pt in the presence of NaCl at pH 5.

FIG. 28 includes the viscosity of 75HA-Lys-Pt in the presence of sodium citrate at pH 5.

FIG. 29 includes the viscosity of 75HA-Lys-Pt in the presence of sodium acetate at pH 5.

FIG. 30 includes the viscosity of 75HA-Lys-Pt in the presence of sodium carbonate at pH 5.

FIG. 31 includes the viscosity of 75HA-Lys-Pt in the presence of sodium citrate and trehalose at pH 5.

FIG. 32 includes the viscosity of 75HA-Lys-Pt in the presence of sodium acetate and trehalose at pH 5.

FIG. 33 includes the viscosity of 75HA-Lys-Pt in the presence of sodium carbonate and trehalose at pH 5.

FIG. 34 includes the viscosity of 75HA-Lys-Pt in the presence of Chelex 100 at pH 5.

FIG. 35 includes the synthetic scheme of HA-PEG-DOTAGA conjugates. a. DMF, TEA, 50° C.; b. TFA, 0° C.; c. NaBH₃CN, acetate buffer (pH 5.6), 50° C.

FIG. 36 includes the chromatograms of sodium hyaluronate (solid line) and HA-PEG-DOTAGA conjugate (dashed line) generated by a GFC with a RI detector.

FIG. 37 includes the synthetic scheme of HA-DBCO-DOTA-Tm conjugate. a. DMF, TEA, 50° C.; b, water, pH 6.0, 80° C.; c. water, DMTMM, pH 4.5, 30° C.

FIG. 38 includes the UV absorbance spectra of Arsenazo III solution (25 nM) prepared in the presence of free Tm(III), DOTA-Tm and HA-DBCO-DOTA-Tm solution.

FIG. 39 includes the FTIR spectra of DOTAGA, DOTA-azido and DOTA-Tm.

FIG. 40 includes the UV/Vis spectra of DOTA-azido (dashed), DOTA-Tm (solid) and Tm(III) (dash-dot).

FIG. 41 includes the in vitro release profiles of Tm(III) from HA-DBCO-DOTA-Tm and HA-Tm conjugates in PBS and 10% FBS/PBS (pH 7.4) at 37° C. FIG. 42 includes the plasma concentration of Tm(III) after subcutaneous injection of HA-DBCO-DOTA-Tm or DOTA-Tm (0.03 mg/kg on Tm(III) basis) into lower cheeks of rats.

FIG. 43 includes the synthesis of HA-pHP-Lys conjugate.

FIG. 44 includes the synthesis of HA-Homo-Lys conjugate.

FIG. 45 is molecular modeling of a 5 member ring chelate of cisplatin with acetyl lysine. C2 corresponds to the alpha carbon of lysine and C3 to the first carbon of the lysine alkyl amine side chain.

FIG. 46 is a molecular modeling of a 6 member ring chelate of cisplatin with acetyl homolysine. C2 corresponds to the alpha carbon of homolysine and C3 to the first carbon of the homolysine alkyl amine side chain.

FIG. 47 depicts PK results for a rabbit toxicity study of lyophilized HA-Pt.

FIG. 48 depicts rabbit toxicity data for low dose HA-Pt.

FIG. 49 depicts PK of Rabbit toxicity study of lyophilized HA-Pt.

FIG. 50 depicts total and free Pt levels for a dog study.

### DETAILED DESCRIPTION OF PREFERRED EMBODIMENT

One aspect of the present invention is directed to conjugates that comprise a linear polymer carrier, a linker and one or more drugs or imaging agents, as well as pharmaceutical compositions that include such conjugates. Another aspect of the invention is directed to formulations and processes for lyophilization of hyaluronan conjugates. Another aspect of the invention is directed to methods for treating and/or inhibiting cancer by administering the compositions of the invention.

Hyaluronan-Linker-Platinum (HA-Linker-Pt) Drug Conjugates

One embodiment of the conjugates of the present invention is directed to a drug conjugate comprising hyaluronan (HA), a chelated platinum (Pt), and a linker joining the hyaluronan and platinum. It is believed the unexpected toxicity of the HA-Pt conjugates discussed in the Background, above, was due to the altered deposition of the HA bound platinum, the prolonged retention in the liver during HA metabolism, and the rapid release of the Pt-diaqua from the conjugate. HA-linker-Pt conjugates of the present invention have extended release half-life and improved safety both in the presence and absence of salts. It was believed that slower and acid-sensitive release would decrease burst release after injection, prolong release into tissues, and alleviate toxicities.

The linker preferably includes an amine, amide or carboxyl group capable of chelating the platinum. Preferably the linker is capable of chelating the platinum in the II oxidative state. The linker may include at least two groups selected from the group consisting of amine, amide and carboxyl. Each of the groups may be the same or different. For example, the linker may include an amide group and a carboxyl group, or the linker may include 2 amino groups.

The linker may include a modified amino acid capable of chelating the platinum, preferably in the II oxidative state, and in certain embodiments contains one or two modified amino acids. Derivatives of lysine, such as N-acetyl lysine, have been found to be particularly suitable for use in the hyaluronan-linker-Pt conjugates of the present invention. Other exemplary modified amino acids include derivatives of glutamic acid, such as N-acetyl glutamic acid, as well as L-β-homolysine and homolysine derivatives, such as N-acetyl L-homolysine.

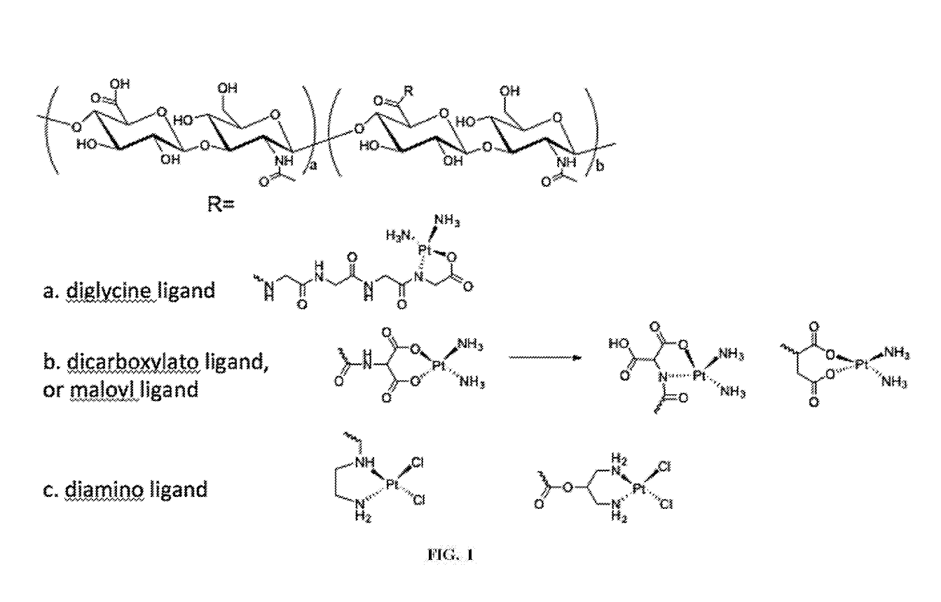

In other embodiments, the linker comprises a ligand selected from the group consisting of diglycine, dicarboxylato, maloyl, and diamino, as depicted in FIG. 1, or comprises HOOC—X—NH—Y, wherein X may be CH₂, CH₂Z, CHZCH₂, wherein Z is an aliphatic group, and Y is an acetyl, ethyl or an aliphatic chain. Suitable aliphatic chains may be cyclic or non-cyclic, and saturated or unsaturated. The aliphatic groups can be CH₂, (CH₂)n, CH═CH, and CH₂(CH═CH)CH₂, or ring structures that contain a 5- or 6-membered ring.

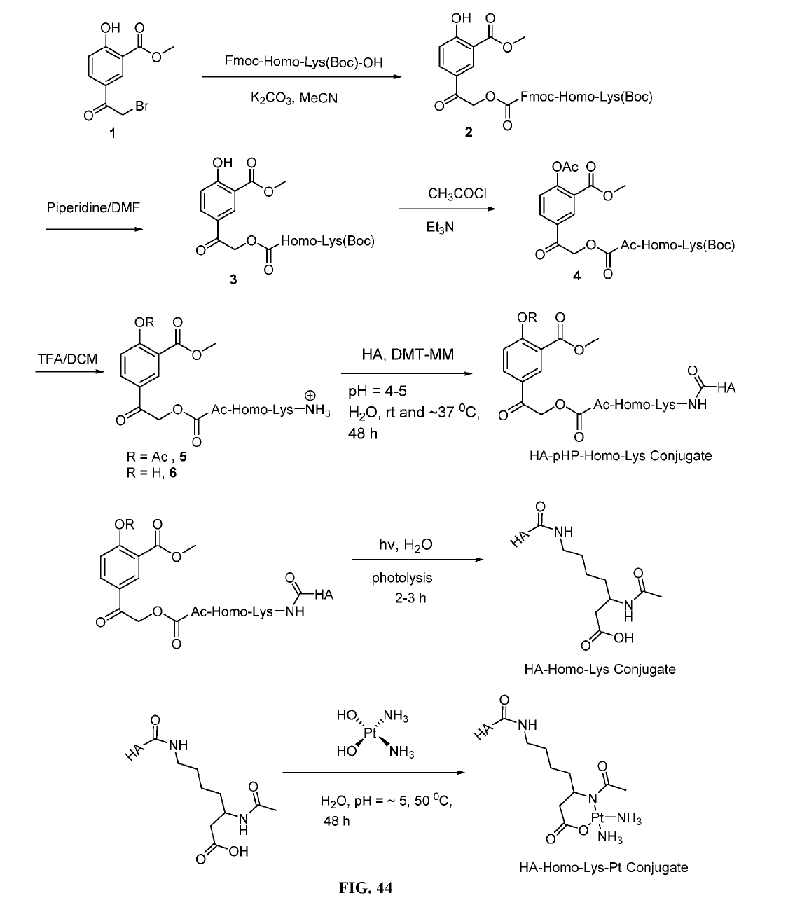

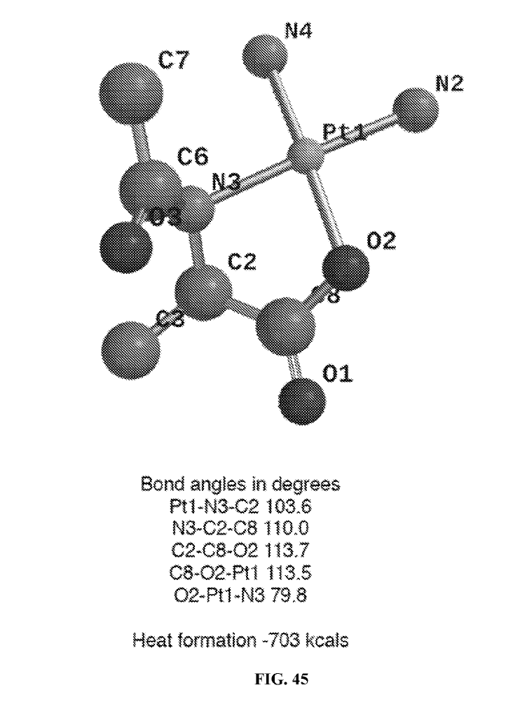

In certain embodiments, the platinum may form a 5 member ring or a 6 member ring with the ring forming ligand of the linker. FIGS. 44 and 45 depict examples of 5 and 6 member rings. It was surprisingly found that the 6 member ring is both easier to form, and has faster hydrolysis and release of the platinum, than the 5 member ring. Both of these results were very unexpected. It is believed these unexpected results could be due to relaxed bond stretching in the 6 member ring compared to the 5 member ring.

The linker may include additional elements. For example, the linker may include additional groups to facilitate cleavage from the hyaluronan or to facilitate the linkage between the linker and the hyaluronan. Hydrazine groups may be employed with linkers having a free carboxyl group, such as glutamic acid and aspartic acid and their derivatives.

The linkers may be acid-labile or degradable by enzymes, light and/or temperature. For example, the linker may employ esters, amino acids or amides that are enzymatically degradable. It has been found that drug conjugates of the present invention are degradable by hyaluronidases. The linker may be degraded in the presence of enzymes (e.g. PSMA or matrix metalloproteinases), proteins, proteases, esterases, amidases, peptidase, reducing or hypoxid environment, acidic environment, or chemicals (for example peroxides) present at higher concentrations in tumor or lymphatic tissues than some other tissues. In another aspect of this embodiment, the linker may be degraded by light from an external source, for example a laser light that is between 500 and 1200 nm (e.g. 500, 550, 600, 650, 700, 750, 800, 850, 900, 950, 1000, 1050, 1100, 1150, or 1200 nm and all values and ranges therebetween). In another aspect of this embodiment, the linker may be degraded when the tissue is at a higher temperature than commonly found in the body, for example with focused heating from ultrasound, magnetic sources, laser or infrared light irradiation, or an applied heating source (e.g. heating pad, perfusions of heated fluids, heated bath).

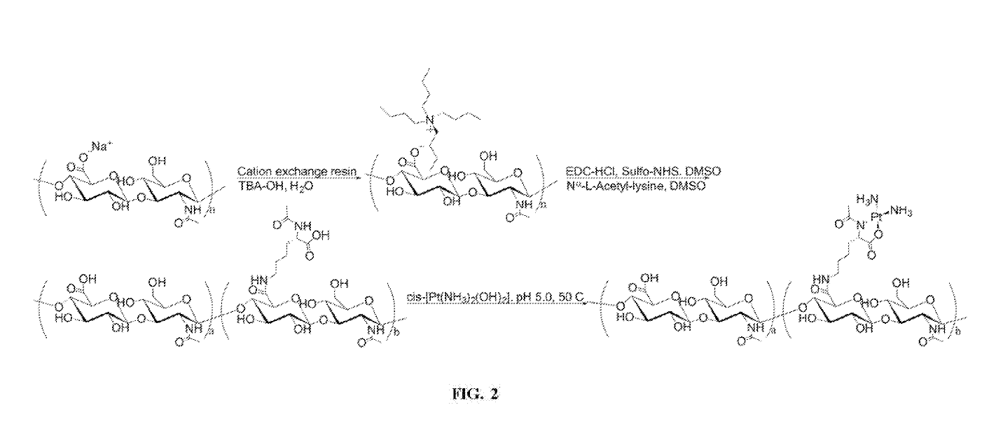

The linkers may be attached to one or more carboxyl groups on the hyaluronan molecule, as shown in FIG. 2, and/or to the linking end/reducing end of the hyaluronan molecule, as shown in FIG. 3. Attachment at the reducing end is preferred for very small hyaluronan molecules, <15 kDa, as it should not interfere with cell recognition. The carboxyl linking scheme is used for hyaluronans 15 kDa and larger, including hyaluronans from 15 to 300 kDa. Attachment of the linker to the carboxyl group allows much more drug to be loaded, and it has been found enzymatically that hyaluronidases can still recognize even the more fully loaded drug conjugates. It is also possible during conjugation of the linker to the carboxyl groups for some linkers to conjugate on the reducing end of the hyaluronan, such that a drug conjugate may have linkers attached to both the reducing end and one or more carboxyl groups. It is well known that amines can react with reducing sugars spontaneously at physiological and acid pHs to produce hydrazone that can release, be biodegraded, or spontaneously reduced to amine linkages.

The drug conjugate can preferably be loaded with drugs from 0.1% to 40% w/w (e.g. 0.1, 0.5, 1, 2, 4, 6, 8, 10, 12, 15, 18, 20, 23, 25, 30, 35, 40% w/w and all values and ranges therebetween), preferably from 1 to 20% w/w. In one aspect, the drug conjugate compositions comprise 10% to 200% (e.g., 10, 15, 20, 25, 30, 35, 40, 45, 50, 55, 60, 65, 70, 75, 80, 85, 90, 95,100, 110, 120, 130, 140, 150, 160, 170, 180, 190, and 200% and all values and ranges therebetween) of the maximum tolerated dose of cisplatin. MTD is determined as a maximum dose that does not result in acute toxicity, immediate death, more than 40% weight loss, or can be a level that does not produce severe side effects or the side effects are manageable or partially reversible.

In certain embodiments, the platinum forms part of a drug. The drug may be selected from the group consisting of cisplatin, carboplatin and oxaliplatin. The platinum may be in the II oxidative state.

The hyaluronan carrier is preferably a linear polymer of 5 kDa to 10000 kDa (e.g. 5, 10, 15, 20, 25, 30, 35, 40, 45, 50, 55, 60, 65, 70, 75, 80, 90, 100, 110, 120, 130, 150, 175, 200, 300, 400, 500, 600, 700, 800, 1000, 1500, 2000, 3000, 4000, 5000, 6000, 7000, 8000, 9000 or 10000 kDa and all values and ranges therebetween). The hyaluronan carrier of the present invention is preferably 6 kDa to 300 kDa, more preferably from 20 kDa to 200 kDa. The HA-linker-Pt conjugates may form a nanoconjugate. A nanoconjugate generally has a size less than 1 micron. Nanoconjugates of the present invention generally have a size less than 1 micron, more preferably less than 400 nm, less than 200 nm, less than 150 nm, and less than 50, and all values and ranges therebetween.

The HA-linker-Pt conjugates of the present invention are configured for percutaneous, subcutaneous, intratumoral, intradermal, peritumoral, submucosal, intravascular, intralymphatic, nodal, intratissue, intravenous, intraorgan, transdermal, intramucosal, intramuscular, intrathecal, intrapleural, intravesical, intralesional, intra-articular, intra-lobular, bronchoscopic, or endoscopic administration, administration via a catheter, or any other means of injection or insertion into an anatomic site. The HA-linker-Pt conjugates of the present invention can be tailored for sustained release and preferential intralymphatic or tumoral accumulation after administration, preferably via subcutaneous, peritumoral or intratumoral injections. The present invention contemplates administering the HA-linker-Pt conjugates, and compositions containing such conjugates, by any such methods for purpose of treating and/or inhibiting cancer. The composition may include a pharmaceutically acceptable diluent. Further, the composition may be co-administered with one or more other active agents and/or may be administered as an adjuvant.

The HA can have a charge, which may be anionic, neutral or cationic. Preferably, the charge is anionic or neutral. More preferably, the charge when measured by zeta potential is between −10 and −100 mV, and more preferentially between −20 and −80 mV, and yet more preferentially between −30 and −50 mV.

In one embodiment, the conjugate is highly water soluble, in excess of 10 mg/mL, more preferred in excess of 20 mg/mL, and more preferred in excess of 30 mg/mL and even more preferred in excess of 50 mg/mL, and even more preferred in excess of 70 mg/mL and even more preferred in excess of 200 mg/mL and even more preferred in excess of 300 mg/mL.

The compositions of the present invention may include, in addition to the drug conjugate, a second hyaluronan having a molecular weight greater than 600 kDa. The second hyaluronan is not part the drug conjugate but is added to the composition comprising the drug conjugate. Although the second higher molecular weight HA is not linked to any drug when it is added to the composition, some of the drug conjugated to the smaller HA molecules could spontaneously transfer from the drug conjugate to the second higher molecular weight HA. Thus, the second higher molecular weight HA is linked to no drug, or substantially less drug than the smaller HA molecules onto which the drug is loaded.

The use of higher molecular weight hyaluronan can slow or delay release, normalize PK between patients (i.e. reduce patient to patient variability), reduce maximum plasma concentration of drug (Cmax), and reduce hepatic toxicity. Typically the second hyaluronan is a high molecular weight hyaluronan, typically having a molecular weight from 600 kDa to 6MA, preferably over 600 kDa, preferably over 1 MDa and more preferably over 2 MDa. Suitable high molecular weight HA include 600 kDa, 800 kDa, 1 MDa, 1.3 MDa, 1.6 MDa, 2 MDa 4 MDa, 6 MDa, and all values and ranges therebetween. The concentration of the high molecular weight hyaluronan is typically 0.1 to 2% wt, preferably 0.5 to 1.5% wt, and more preferably 1 to 1.5% wt in the drug conjugate composition.

The benefits of a higher molecular weight hyaluronan must be considered against the difficulties in achieving high drug loading with the higher molecular weight hyaluronan, which is difficult to work with synthetically because of is high viscosity. As discussed above, a lower molecular weight hyaluronan, less than 300 kDa, preferably less than 200 kDa, is preferred for use in the drug conjugate to achieve high drug loading. To obtain the benefits of both the high molecular weight and lower molecular weight hyaluronan, lower molecular weight hyaluronan can be loaded with high levels of the drug, and then mixed with the high molecular weight hyaluronan described above, to obtain the benefits of longer release and better safety associated with high molecular weight hyaluronan.

The impact of hyaluronans with various molecular weights are shown in the Examples, including Examples 71, 72, and 74, which assessed both mouse and rabbit toxicity, and rabbit PK. Hyaluronans having a molecular weight of 2 MDa have been administered to several canine patients, as discussed in Example 75.

The compositions of the present invention comprising the HA-linker-Pt conjugates may also comprise excipients, such as salts and sugars, as described with respect to the lyophilization formulations.

It was surprisingly found that the conjugates of the present invention are substantially less viscous, preferably more than 15% less viscous, in the presence of a sugar excipient such as mannitol, sucrose and trehalose than a drug conjugate without the presence of an excipient. For example, with the addition of sugars, therapeutically relevant concentrations of drugs can be formulated for administration through a small needle, e.g. 23 ga or less, or 25 ga, 27 ga, or 30 ga, and all values and ranges therebetween. The concentration of the sugar in the composition can be 0.1 to 25% wt (e.g. 0.1, 0.5, 1, 2, 4, 6, 8, 10, 12, 14, 16, 18, 20, 22, 25% wt and all values and ranges therebetween). The excipient can be mixed, vortexed, or nutated with the drug conjugate for 1 minute to 24 hours (e.g., 1, 5, 10, 20, 30, 45, and 60 minutes, and 2, 4, 6, 8, 10, 12, 14, 16, 18, 20, 22, and 24 hours and all values and ranges therebetween).

It was also surprisingly found that the conjugates of the present invention are substantially less viscous after addition of a monovalent or a polyvalent cation. The cation may be added by the addition of an excipient consisting of a cation and a corresponding counterion. In one embodiment, the drug conjugate is substantially less viscous in the presence of an excipient such as sodium carbonate, sodium acetate, sodium citrate, sodium Chelex® 100, calcium chloride, calcium acetate, calcium lactate, magnesium chloride, magnesium acetate and magnesium lactate than a drug conjugate without the presence of the aforementioned excipient. The concentration of the salt in the composition can be 1 to 50 mM (e.g. 1, 2, 4, 6, 8, 10, 12, 15, 20, 25, 30, 35, 40, 45, 50 mM and all values and ranges therebetween). The excipient can be mixed, vortexed, or nutated with the drug conjugate for 1 minute to 24 hours (e.g., 1, 5, 10, 20, 30, 45, and 60 minutes, and 2, 4, 6, 8, 10, 12, 14, 16, 18, 20, 22, and 24 hours and all values and ranges therebetween).

The viscosity of the composition after the addition of the salt and/or sugar excipients is preferably less than 2000 cP, and more preferable a lower viscosity (e.g. less than 1500, 1000, 700, 500, 400, 300, 200, 150, 100, 75, 50, and all ranges and values therebetween), or even more preferably less than 25 cP. In certain embodiments, the composition comprises both a sugar and salt and has a viscosity from 5 to 200 cP.

The less viscous conjugate composition can be more easily administered to the body through a delivery device, such as a needle, catheter, or other devices used to place drugs into the body via the injection routes described herein. The viscosities described herein can apply to the composition comprising the drug conjugate in a solution that includes sufficient drug for safe administration or reasonable therapeutic effect after one or more injections into the body over a treatment session.

Additional details of the formulations described with respect to the lyophilization formulations, discussed below, can also apply to the compositions of the present invention comprising HA-linker-Pt conjugates. For example, the compositions of the present invention many include the HA-linker-Pt conjugate at concentrations (on a platinum drug basis) from 1 to 13 mg/mL, preferably from 5 to 10 mg/mL, and more preferably from 6 to 8 mg/mL. Further, the pH range for the formulations compositions of the present invention is typically from 4 to 7.5, preferably 4.5 to 6.5, and more preferably 5 to 6.

Formulations and Methods for Lyophilization

One aspect of the present invention is a formulation and method for lyophilizing a hyaluronan conjugate.

In one such embodiment, the hyaluronan conjugate is a hyaluronan-drug conjugate. The conjugate preferably contains a platinum drug and may be, for example, the hyaluronan-linker-platinum conjugate described above, or a hyaluronan-platinum conjugate, such as the hyaluronan-cisplatin conjugate disclosed in U.S. Pat. No. 8,088,412, which is incorporated by reference for its disclosure relating to hyaluronan-platinum conjugates (HA-Pt), including a hyaluronan-cisplatin conjugate. The HA-linker-Pt and HA-Pt conjugates are collectively referred to as hyaluronan platinum conjugates. Although hyaluronan platinum conjugates are discussed herein, the lyophilization formulations and methods may also be applied to hyaluronan conjugates comprising other drugs or other compounds, such an imaging agents.

The lyophilization formulations comprise excipients that may include one or more salts, one or more sugars, or a combination of salt and sugar. The salt prevents the formation of diaqua products by converting the aquated platinum back to cisplatin, or other applicable platinum-containing drug. The sugar acts as a cryoprotectant to make the formulation stable for lyophilization and allow the drug conjugate to be rehydrated in a reasonable amount of time.

Early experiments showed that salt was very destabilizing of the HA-Pt conjugates, causing the platinum, in the form of cisplatin, to fall off the carrier. Further, experiments with sugar excipients caused the formation of diaqua platinum. However, attempts to lyophilize without salt made the drug conjugate hard to rehydrate. Thus, it was very unexpected that controlled amounts of salt prior to lyophilization under controlled conditions could actually stabilize the hyaluronan platinum conjugates, resulting in a lyophilized product that would rehydrate well without diaqua platinum products and with the drug still on the conjugate.

Suitable concentrations of the excipients in the formulation include 1 to 20% wt of sugars, more specifically, 1, 2, 3, 4, 5, 6, 7, 8, 9, 10, 11, 12, 13, 14, 15, 16, 17, 18, 19 and 20% wt, and all values and ranges therebetween. The ranges of concentrations of salts that are suitable include 0.01 to 0.9% wt, more specifically, 0.01, 0.02, 0.05, 0.1, 0.2, 0.3, 0.4, 0.5, 0.6, 0.7, 0.8 and 0.9% wt and all values and ranges therebetween. Suitable salts include sodium and calcium salts, including calcium chloride, calcium acetate, calcium lactate, sodium chloride, sodium citrate, sodium acetate, sodium Chelex® 100, and sodium carbonate. Sodium chloride has been shown to be particularly suitable in the lyophilization process of the present invention. Suitable sugars include trehalose, mannitol and sucrose. Trehalose has been shown to be particularly suitable in the lyophilization process of the present invention.

Formulations consistent with the present invention include the hyaluronan platinum conjugate at concentrations (on a platinum drug basis) from 1 to 13 mg/mL, preferably from 5 to 10 mg/mL, and more preferably from 6 to 8 mg/mL. The pH range for the formulations is typically from 4 to 7.5, preferably 4.5 to 6.5, and more preferably 5 to 6.

It was found that lyophilization stabilizes the formulation and prevents the formation of potentially toxic free platinum such as monoquated platinum and di-aquated platinum. The lyophilization process preferably includes freezing the formulation, followed by drying the formulation.

One embodiment of the lyophilization process of the present invention includes gradual freezing the formulation at about −80° C., followed by a multi-step drying process. More specifically, the lyophilization process preferably comprises freezing the formulation at a temperature from −60° C. to −85° C., preferably −80° C. Preferably the formulation is frozen in a −80 freezer, which will be understood by one in the art to refer to a freezer set at −80° C., but in which the temperature may generally vary from −60° C. to −85° C. Such freezing step can typically range from 5 min to 72 hours, preferably 2 to 48 hours, and more preferably 4 to 24 hours. Alternatively, the formulation may be flash frozen in less than one hour, preferably in liquid nitrogen, followed by a multi-step drying process.

The multi-step drying process preferably comprises a primary drying process and a secondary drying process.

The primary drying process is typically conducted a temperature ranging from −90 to −15° C., and more preferably −80 to −30° C. The duration is typically 30 to 96 hours, preferably 48 to 96 hours, and more preferably 72 to 96 hours.

The secondary drying process is typically conducted at a temperature from 1 to 50° C., preferably from 2 to 25° C. and more preferably from 2 to 10° C. The secondary drying process can typically last 2 to 24 hours, preferably 2 to 12 hours, more preferably 2 to 8 hours.

In certain embodiments, a two-step annealing process precedes the primary drying step. The annealing process typically includes a first step with annealing temperatures typically from −20 to −1° C., preferably −10 to −2° C. The first annealing step typically ranges from 1 to 24 hours, preferably 2 to 12 hours. The second annealing step typically utilizes temperatures from −90 to −30° C., preferably −80 to −40° C. The second annealing step typically ranges from 1 to 24 hours, preferably 2 to 12 hours, prior to the primary drying step.

The drying steps may be conducted in a freeze dryer. Any modern shelf freeze drying would be capable of conducting a programmable multistep freeze drying procedure. Exemplary lyophilization processes are described in Examples 68 and 69, below.

The lyophilized conjugates may be stored at a temperature near −20° C. for up to 6 months, or longer. The conjugates are then rehydrated. Suitable media for rehydration include water, which may include salt, sugar and/or high molecular weight HA. The sugar is typically <10% wt, more preferably <5% wt. The salt is typically <0.9% wt, more preferably <5% wt. Exemplary salts and sugars include sodium chloride and trehalose, as well as the salts and sugars discussed with respect to the lyophilization formulations. The rehydration medium may comprise water with high molecular weight HA and with or without salt and/or sugar. Suitable high molecular weight hyaluronans and concentrations include those discussed above with respect to the compositions of the HA-linker-Pt conjugates. As discussed in more detail above, the presence of high molecular weight HA stabilizes the hyaluronan-Pt conjugate by slowing the formation of free platinum. The lyophilized conjugates can be rehydrated in relatively short periods of time, including less than 30 seconds when the rehydration medium does not include high molecular weight HA, and approximately 10 minutes when high molecular weight HA is present.

Pharmacokinetics and toxicity studies were conducted in mice, rabbits and dogs to evaluate the tolerability of HA-Pt. Pharmacokinetic studies help to identify the formulation candidates with more favorable plasma concentrations v. time curve, lower Cmax and longer retention. Toxicity studies include MTD determination, body weight monitoring, survival rate determination and evaluation of changes in blood work, including complete blood counts and liver enzymes.

Hyaluronan-Linker-Metal Complex Conjugates

Another embodiment of the conjugates of the present invention is directed to a conjugate comprising hyaluronan, a metal complexing ligand and a linker joining the hyaluronan and the metal complexing ligand. The metal complexing ligand is preferably 1,4,7,10-tetraazacyclododecane-1,4,7,10-tetraacetic acid (DOTA), ethylenediaminetetraacetic acid (EDTA) or DTPA (diethylenetriaminepentaacetic acid), most preferably of DOTA. In certain embodiments the metal is complexed to the metal complexing ligand.

The metal may function as a drug or imaging agent. The imaging agent may provide for contrast in one or more imaging techniques, including but not limited to: PET, MRI, photoacoustic imaging, fluorescence imaging, ultrasound, CAT, and SPECT. The drug is preferably a chemotherapeutic drug. Suitable metals include lanthanides, such as thulium, and radiometals that emit positrons, such as ⁶⁴Cu, ^{66,68}Ga and ⁸⁶Y.

The linker may comprise polyethylene glycol or dibenzocyclooctyl (DBCO). Any linker with an amino-terminus or a hydroxyl-terminus can be linked to DOTA. The linker may comprise additional elements, including groups that connect the linker to the hyaluronan or metal complexing ligand, such as a hydrazide or amino group. In certain embodiments, the DOTA is 2,2′,2″-(10-(2,6-dioxotetrahydro-2H-pyran-3-yl)-1,4,7,10-tetraazacyclododecane-1,4,7-triyl)triacetic acid (DOT-GA), although any DOTA with functional groups for conjugation can be used.

The hyaluronan used in, compositions comprising, methods of administering and other descriptions above with respect to the HA-linker —Pt conjugate and hyaluronan-platinum drug conjugates may also be applied to the HA-linker-metal complexing ligand.

Certain aspects of the present invention are illustrated by the following non-limiting examples.

### EXAMPLES

Unless noted, all reagents were of highest grade available from Fisher Scientific (Pittsburgh, Pa.). The MDA-1986 human oral squamous carcinoma and the 4T1.2-neu murine breast cancer cell lines were kindly provided by Dr. Jeffery Myers (M.D. Anderson Cancer Center, Houston, Tex.), and Dr. Zhaoyang You (The University of Pittsburgh, Pittsburgh, Pa.), respectively. The human melanoma cell line was purchased from ATCC (Manassas, Va.). Water for all aqueous solutions was ASTM Type 1. All reactions were conducted at ambient temperature, ca. 20° C., unless noted otherwise. Animal procedures were approved by the University of Kansas Institutional Animal Care and Use Committee. Sprague Dawley rats, Balb/C and Nu/Nu mice were purchased from Charles River Laboratories (Wilmington, Mass.).

### Example 1

### Synthesis of Hyaluronic Acid-Tetrabutylamonium (HA-TBA) Salt

Sodium hyaluronate (35- or 75-kDa, Lifecore Biomedical, Chaska, Minn.) was dissolved in water (1 g in ca. 300 mL) and then stirred overnight with 10 grams of cation exchange resin (Dowex 50W-X8, Bio-Rad, Hercules, Calif.). After filtration, the filtrate was neutralized with tetrabutylammonium hydroxide solution (TBA-OH, 55-60% w/v in water), and then freeze dried to obtain an orange to pink powder. The products were named 35HA-TBA and 75HA-TBA to reflect the molecular weights of the HA used.

### Example 2

### NMR Characterization of HA-TBA

¹H-NMIR (400 MHz, D₂O) δ (ppm): 4.46 (d, J=6.8 Hz, 1H), 4.36 (d, J=6.8 Hz, 1H), 3.74-3.25 (m, 10H), 3.12-3.08 (t, J=8.4 Hz, 8H), 1.92 (s, 3H), 1.60-1.52 (tt, J=8 Hz, 8H), 1.27 (qt, J=7.2 Hz, 8H), 0.85 (t, J=7.2 Hz, 12H).

### Example 3

### Synthesis of HA-Na-Acetyl-L-lys (HA-Lys)

Two hundred milligrams of HA-TBA was dissolved in 10 mL of dimethyl sulfoxide (DMSO) and after 10 minutes the solution turned clear and homogenous. The polymer was activated with 123.6 mg of N-(3-Dimethylaminopropyl)-N′-ethylcarbodiimide hydrochloride (EDC.HCl) and 140 mg of N-hydroxysulfosuccinimide (sulfo-NHS), and after 2 hours of stirring, 121.3 mg of N^{α}-Acetyl-L-lysine (Chem-Impex International Inc., Wood Dale, Ill.) in 4 mL of DMSO was added and stirred for another 22-24 hours. The product was dialyzed (10,000 MWCO, Fisher Scientific) against water for 4 hours, 100-mM sodium chloride for 2 hours, followed by two water changes over 18 hours. The product was filtered (0.22-μm PES syringe filter) and concentrated using a centricon filter (10,000 MWCO, Fisher Scientific). The products were named 35HA-Lys and 75HA-Lys to reflect the molecular weights of the HA used.

### Example 4

### NMR Characterization of HA-Lys

¹H-NMR (400 MHz, D₂O) δ (ppm): 4.57-4.29 (m, 2H), 4.06 (dd, J=5.6 Hz, J=7.6 Hz, 1H), 3.74-3.25 (m, 10H), 1.95 (s, 3H), 1.94 (s, 3H), 1.66-1.58 (m, 2H), 1.43 (tt, J=6.4 Hz, 2H), 1.12 (m, 2H).

### Example 5

### Synthesis of HA-cis-[Pt(NH₃)₂(N^{α}-Acetyl-L-Lysine)] (HA-lys-Pt)

FIG. 2 shows the synthesis of HA-cis-[Pt(NH₃)₂(N^{α}-Acetyl-L-Lysine)] (HA-lys-Pt). Cisplatin (60 mg, 200 μmol, Qilu Pharmaceutical Inc., Shandong, China) was suspended in 3 mL of ca. 35° C. water and then stirred with AgNO₃ (68 mg, 400 μmol) for 1 day at ca. 20° C. in the dark. The AgCl₂ precipitate was removed by centrifuging (4,000 rpm, 10 minutes), and the supernatant was filtered (0.22-μm nylon syringe filter) to obtain cis-[Pt(NH₃)₂(OH)₂].

The HA-lys (35HA-Lys or 75HA-Lys, 125 mg) was dissolved in 15 mL of water, to which 815 μL of cis-[Pt(NH₃)₂(OH)₂] (27 mg/mL on cisplatin basis) was added, and the pH of the mixture periodically adjusted to ˜5.0 using 1-N NaOH. The reaction proceeded at 40° C. for 1 day and 50° C. for 2 days in the dark (FIG. 2). The resulting solution was filtered (0.22-μm nylon syringe filter) and then dialyzed (10,000 MWCO) against water for 4 hours, sodium phosphate solution (100-mM NaCl, 10-mM NaH₂PO₄, pH 7.4) for 2 hours, and two water changes over 18 hours.

### Example 6

### NMR Characterization of HA-lys-Pt

¹³C-NMR (500 MHz, D₂O) δ (ppm): 190.2, 178.0, 174.9, 174.0, 103.1, 100.5, 82.4, 79.8, 76.2, 75.3, 73.5, 72.4, 68.4, 65.2, 60.5, 54.3, 42.7, 36.0, 28.4, 24.1, 22.5, 21.7.

### Example 7

### Synthesis of HA_{6.4}-N^{α}-Acetyl-L-lys (HA_{6.4}-lys)

HA (6,400 Da, 1 g) was dissolved in 100 mL of borate buffer (0.1 M, pH 8.5) with 0.4-M NaCl, followed by the addition of N^{α}-acetyl-lysine (970.5 mg, 33 eq.). The mixture was stirred at 40° C. for several minutes, until the lysine was dissolved completely, and sodium cyanoborohydride (324 mg, 33 eq.) was added to the solution. The reaction proceeded at 40° C. for 3 days. The product was purified by dialysis (1,000 MWCO) against two changes of 0.5-M NaCl and two changes of water within 48 hours, and then concentrated to 25 mg/mL using centrifugal filters (PES, 3000 MWCO). One milliliter of concentrated solution was filtered through a PES syringe filter (0.22 μm) and dried under reduced pressure before the substitution degree was determined by ¹H-NMR.

### Example 8

### NMR Characterization of HA_{6.4}-lys

¹H-NMR (400 MHz, D₂O) δ (ppm): 4.46-4.36 (m, 2H), 4.12 (d, J=3.3 Hz, 1H), 4.05-3.99 (m, 3H), 3.74-3.25 (m, 10H), 3.07 (dd, J=9.7 Hz, J=12 Hz, 1H), 2.98 (t, J=7.9 Hz), 1.91 (s, 3H), 1.75-1.56 (m, 4H), 1.31 (tt, J=7.7 Hz, 2H).

### Example 9

### Synthesis of HA_{6.4}-cis-[Pt(NH₃)₂(N_{α}-Acetyl-L-lysine)] (HA_{6.4}-lys-Pt) Conjugates

FIG. 3 shows the synthesis of HA_{6.4}-cis-[Pt(NH₃)₂(N_{α}-Acetyl-L-lysine)] (HA_{6.4}-lys-Pt) conjugates. HA_{6.4}-lys (375 mg) was dissolved in 11 mL of water, to which 1.4 mL of cis-[Pt(NH₃)₂(OH)₂] (25 mg/mL on cisplatin basis) was added, and the pH of the mixture was monitored and adjusted using 1-N NaOH to keep it at ca. 5.0. The reaction proceeded at 50° C. for 2 days in the dark (FIG. 3). The resulting solution was filtered through a PES syringe filter (0.22 μm) and transferred to dialysis tubing (1,000 MWCO). Final product was purified by dialysis against water 6 hours, sodium phosphate solution (100 mM NaCl, 10 mM NaH₂PO₄, pH 7.4) 6 hours, and two water changes over 18 hours. The loading degree of Pt was determined by inductively coupled plasma mass spectrometry (ICP-MS) to be 6-7% wt on a cisplatin basis.

### Example 10

### NMR Characterization of HA_{6.4}-lys-Pt

¹³C-NMR (500 MHz, D₂O) δ (ppm): 190.3, 178.0, 175.4, 174.9, 174.0, 103.1, 100.5, 82.5, 79.9, 76.2, 75.3, 73.6, 72.5, 68.4, 65.3, 60.6, 54.3, 34.6, 26.3, 22.8, 22.5, 21.8.

### Example 11

### Transmission Electron Microscopy of HA-Lys-Pt

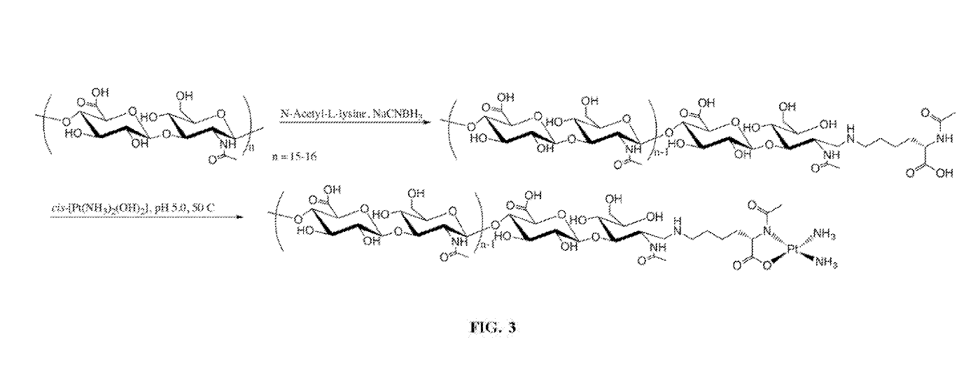

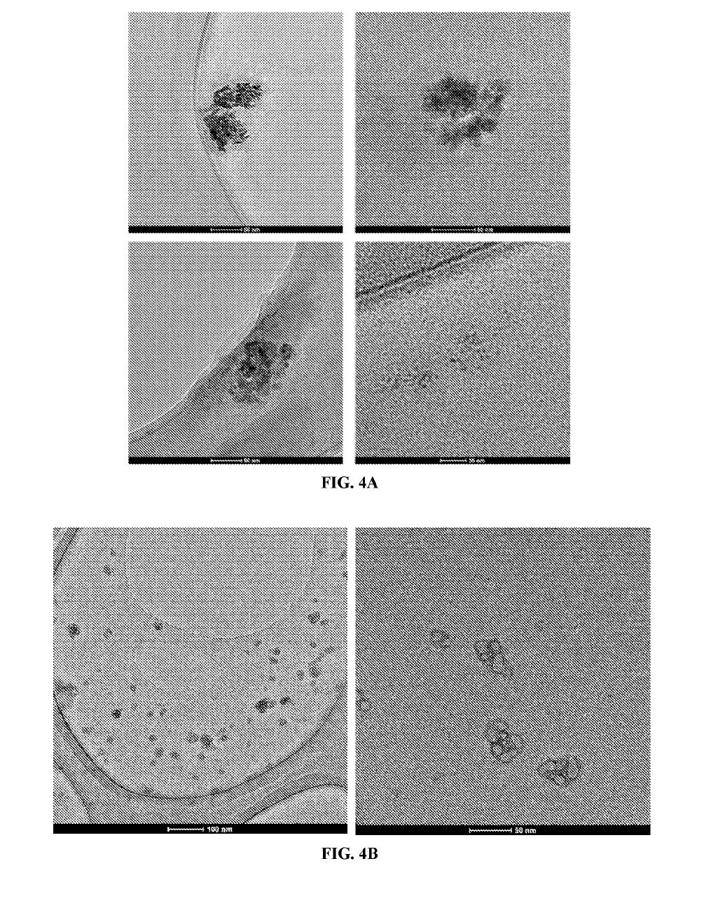

FIGS. 4A and 4B show the transmission electron microscopy images of HA-Lys-Pt (75 kD HA) before and after salt treatment for 2 weeks. The average diameters of HA-Lys-Pt particles ranged from 25 to 50 nm. Salt treated HA-Lys-Pt showed low intensity of Pt due to partial release of the platinum. Intense darkness results from the high electron density of Pt rich area. Salt treated HA-Lys-Pt showed low intensity of Pt due to partial release of the Pt. Reduced clustering of the salt treated nanoparticles may be due to reduced hydrophobicity due to release of Pt and availability of charged chelator groups.

### Example 12

### UV-Vis Spectra of HA-Lys-Pt

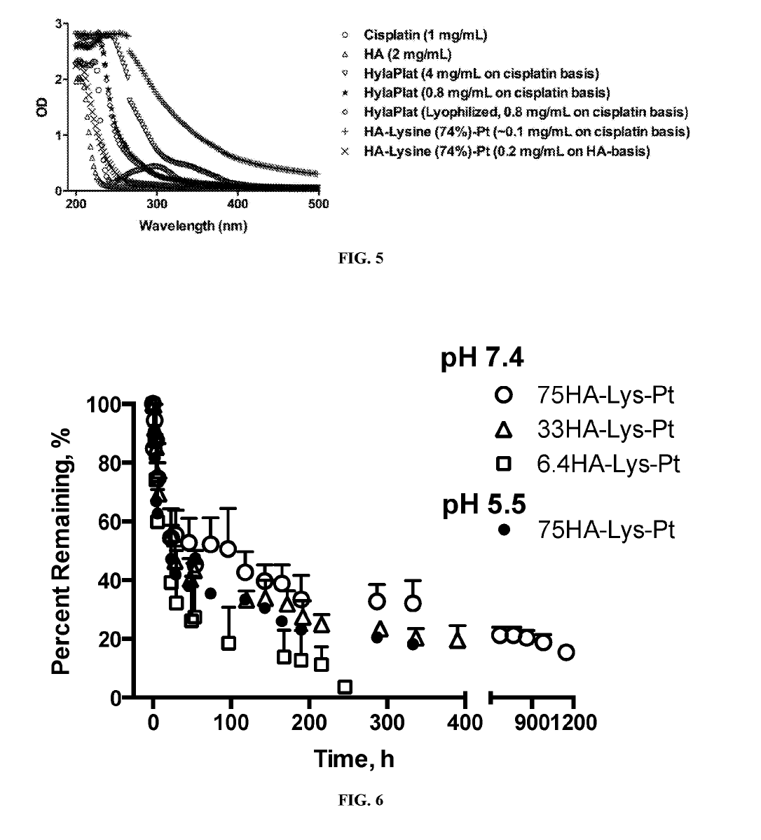

FIG. 5 shows the UV spectra of HA, cisplatin, HylaPlat (HA-Pt nanoparticles with direct Pt conjugation to the carboxylate of HA, and HA-Lys-Pt (75 kDa HA). The spectra were collected using a UV spectrophotometer from 200 to 500 nm.

### Example 13

### Release of Platinum from HA-Lys-Pt

FIG. 6 shows the release of HA-Lys-Pt. A HA-lys-Pt solution (0.1 mg/mL on cisplatin basis) was dialyzed (10,000 MWCO) against phosphate buffered saline release medium (PBS, 12 mM sodium phosphate, 137-mM NaCl, pH 7.4) at 37° C. At pre-determined intervals, 100-4, samples were collected and frozen at −20° C. until analysis. Samples were diluted 10,000-fold using 1% HNO₃ prior to ICP-MS analysis.

The release profiles of platinum from HA-Lys-Pt were evaluated via dialysis against PBS at physiological pH of 7.4, and against acetate buffer at pH 5.5 and 37° C. to simulate the acidic environment of tumor interstitium and cell endosomes. The platinum concentrations in tubing were measured and plotted as the percentage of cumulative drug released against time (FIG. 6). The drug release from 75HA-lys-Pt followed pseudo-first-order kinetics with half-lives of 48 and 24 hours in PBS and acetate buffer, respectively. The slightly more rapid release of Pt(II) ions in an acidic environment was anticipated. This is likely attributed to the acid labile nature of the N^{α}-acetylamido ligand that is protonated easily under acidic condition, followed by de-chelation from the Pt(II). Pt(II) was released from the 75HA-Lys carriers in the form of Pt-diaqua complex. This suggests that the enhanced release of the Pt aqua-active intermediate form at a lower pH may be advantageous to the formation of Pt-purine-DNA adducts in tumor cells.

### Example 14

### Degradation by Hyaluronidase

The hyaluronidase specificity of HA-Lys-Pt was evaluated by incubating 1.5-mg/mL HA-Lys-Pt (concentration on HA basis) with 5-μg/mL hyaluronidase (HAdase, from bovine testes, type I-S, 400-1000 units/mg solid, Fisher Scientific) in sodium phosphate buffer (140-mM NaCl, 16-mM NaH₂PO₄, and 7-mM Na₂HPO₄ at pH 6.4) at 37° C. for up to 24 hours. Timed samples were stored at −80° C. until analysis by size exclusion chromatography (SEC). Samples (N=3) were thawed, diluted 5-fold using mobile phase, and analyzed with a Shodex OHpak SB-804 HQ column using a refractive index (RI) detector.

Conditions were 5-mM ammonium acetate (pH 5.0) mobile phase at 0.8 mL/min, column temperature of 40° C., and injections of 80 μL. Known molecular weights of HA were used as calibration standards.

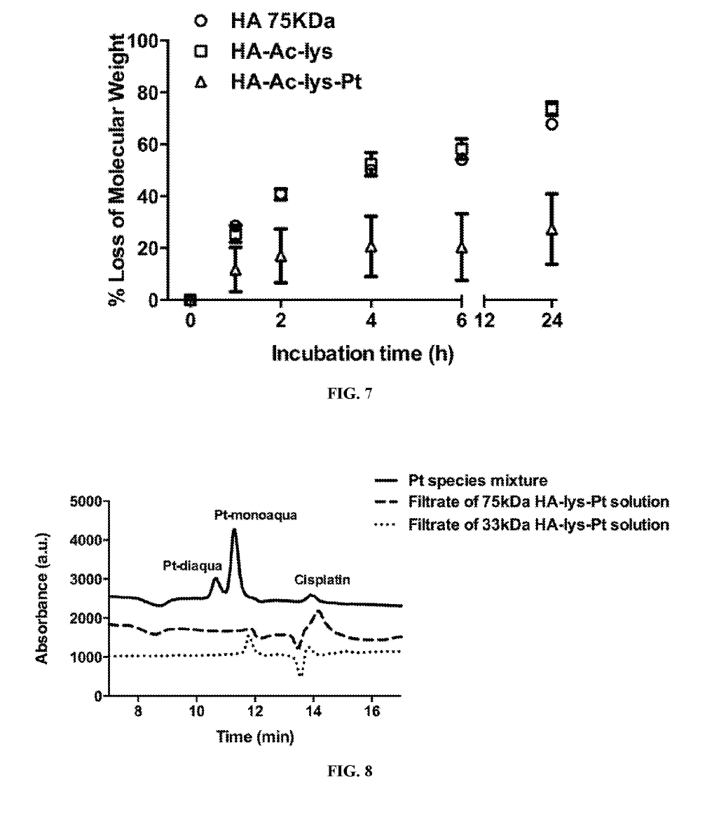

The biocompatibility and biodegradability of HA have led to its usage in dermatology, wound healing, and drug delivery. HA is degraded in vivo by radical attacks, hydrolysis, and enzymatic digestions. The carboxyl group of the glucuronic acid is the recognition site of HA receptors, such as CD44, and hyaluronidase enzymes, such as HYAL1, 2, which cleave HA at the β (1,4) link, thus significant modification may alter its biological interactions and degradability. Both HA-Lys and HA-Lys-Pt maintained similar rates of degradation in the presence of HAdase (p>0.6, FIG. 7). The slower degradation rate of the HA-Lys-Pt compared to the native HA and HA-Ac-Lys may be due to presence of the conjugated platinum. Within the incubation time of 24 hours, only a small amount of drug was liberated, and it is possible that the carrier is only recognized and degraded by the enzyme once the platinum releases from the carrier.

### Example 15

### Purity Analysis by HPLC

Small molecular platinum species including cisplatin, Pt-monoaqua, and Pt-diaqua were quantified by HPLC. The mobile phase was sodium phosphate solution (10-mM, pH 3.0) at a flow rate of 0.25 mL/min on an Alltech® 250 mm×4.6 mm M/M RP8/cation column thermostated at 25° C. The internal standard, a mixture of platinum species containing cisplatin, monoaquated and diaquated platinum, was prepared by dissolving 10 mg of cisplatin in 10 mL of water and stirring for 48 hours. A series of cisplatin solutions in saline were used as calibration standards. Prior to HPLC analysis, 1 mL of HA-lys-Pt solution (1.8 mg/mL on cisplatin basis) was incubated at 4° C. for 4 days, and then separated on a centricon centrifugal filter unit (10,000 MWCO, 4000 rpm for 10 min at 4° C.). The filtrate was immediately injected into the HPLC. The peaks of cisplatin, Pt-monoaqua, and Pt-diaqua were detected by a UV detector at 283 nm.

Pt-diaqua is a potentially toxic impurity in cisplatin-based formulations that may induce hepatic injury and may interfere with a vital nervous system function at the neuromuscular junction. The purity of the HA-Lys-Pt (33 and 75 kDa) solution after being stored at 4° C. for 4 days was tested by HPLC. The level of diaqua was below the limit of quantitation of the assay (18.4 μg/mL). Neither Pt-monoaqua nor Pt-diaqua was detected in the HA-Lys-Pt (FIG. 8).

### Example 16

### Anti-Proliferative Activity of HA-Lys-Pt

MDA-1986 (human head and neck squamous cell carcinoma), 4T1.2-neu (murine breast cancer cells), and A2058 (human melanoma) cells were maintained in Dulbecco's Modified Eagle Medium (DMEM, Corning, Manassas, Va.) with 4.5 g/L glucose, 4.5 g/L L-glutamine, and 10% fetal bovine serum in a humidified incubator at 37° C. and 5% CO₂. Cells were seeded into 96-well plates (3,000 cells/well in 90 μL media) and allowed to attach overnight. Cisplatin, HA-Pt, or HA-lys-Pt was dissolved in water, added to cells (7 concentrations in duplicate; 10-μl addition), and incubated at 37° C. for 72 hours. All doses were on cisplatin basis. Resazurin Blue (Acros Organics, Geel, Belgium) in PBS was added to each well (5-μM final concentration; 10-μL addition) and incubated at 37° C. for 4 hours. Fluorescence (λₑₓ 550 nm, λₑₘ 605 nm) was quantified with a SpectraMax Gemini XS plate-reader (Molecular Devices, Sunnyvale, Calif.). The relative growth of cells incubated with each compound concentration was normalized to vehicle-treated controls (100% proliferation). Data from at least three separate experiments performed in duplicate was analyzed using non-linear regression (GraphPad Prism 5.0) to generate IC₅₀ values.

When cisplatin is directly conjugated to the carboxylate groups of hyaluronan, it forms a HA-Pt ionic and covalent complex that has been evaluated in our previous studies. HA-Pt exhibited a similar anti-proliferative activity as cisplatin against the human head and neck squamous cell carcinoma (HNSCC) cell line, MDA-1986. The in vitro toxicity of the conjugates was due to the release of the active forms of Pt species upon hydrolysis. The cytotoxicity of HA-Pt and HA-Lys-Pt conjugates was likely due to either carrier-mediated endocytosis of the conjugates prior to the cellular release of the drug and/or free drug internalization via passive diffusion following the extracellular cleavage of the drugs.

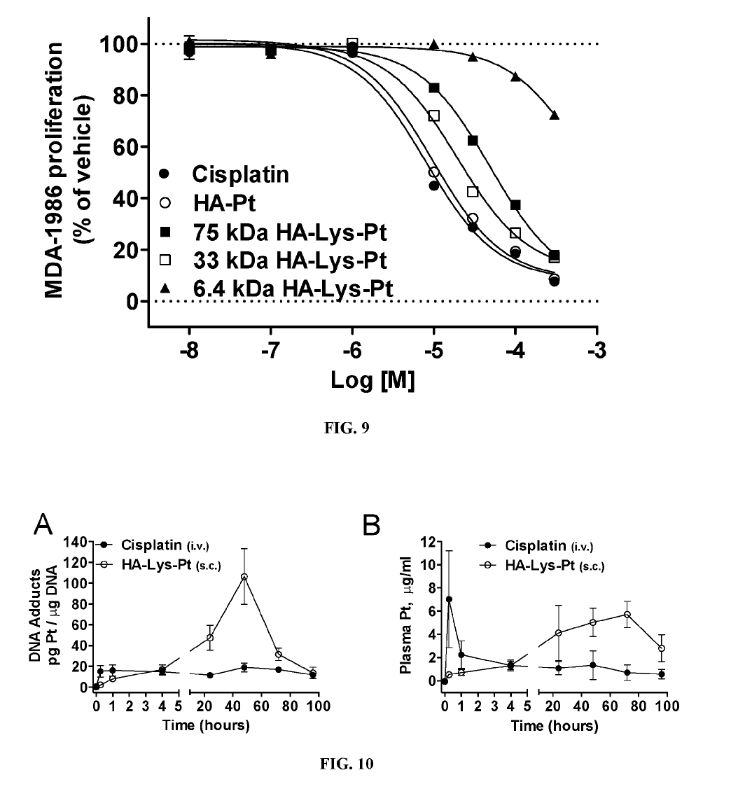

The anti-proliferative effect of HA-Lys-Pt was evaluated using MDA-1986 human head and neck cancer, A2058 human melanoma, and 4T1.2-neu murine breast cancer cell lines (FIG. 9, Table 1). In all three cell lines, the positive control, cisplatin, and HA-Pt show equivalent growth inhibition potencies (IC₅₀≅8 μM), as well as complete inhibition of growth at the highest doses (full efficacy). The 75- and 33-kDa HA-Lys-Pt conjugates also show full anti-proliferative efficacy in MDA-1986 cells, but they are significantly less potent (IC₅₀≅21 and 40 respectively) than cisplatin or HA-Pt. This is attributed to the extended release of the free drug from the conjugate (t_{1/2}=48 hours in PBS) compared to HA-Pt (t_{1/2}=10 hours in PBS). The active Pt species were almost completely cleaved from HA-Pt during the 3-day incubation in cells. However, within the same duration, HA-Lys-Pt partially released the hydrolyzed free drug, resulting in decreased in vitro toxicity. The 6.4-kDa conjugate inhibited cell growth<50% at the highest dose tested (300 μM), and therefore was not evaluated further.

Table 1 shows the anti-proliferative potency of Pt-based anti-cancer drugs in vitro.

TABLE 1

| | Cisplatin | HA-Pt | HA-Lys-Pt (μM) | | |
|---|---|---|---|---|---|
| Cell line | (μM) | (μM) | 6.4 kDa | 33 kDa | 75 kDa |
| MDA-1986 | 8.2 ± 0.6 | 8.4 ± 0.6 | >500^{a} | 20.7 ± 2.9 ^{b, c, d} | 39.8 ± 4.4 ^{b, c} |
| A2058 | 6.8 ± 2.7 | 8.0 ± 3.4 | N.T. | N.T. | 69.8 ± 7.0 ^{e} |
| 4T1.2-neu | 9.4 ± 2.5 | 8.4 ± 2.7 | N.T. | N.T. | 80.1 ± 12.7 ^{f} |

Data are mean IC₅₀ ± S.E.M.
Student's t-test:
^{a}estimated;
^{b} p < 0.001 vs cisplatin;
^{c} p < 0.05 vs HA-Pt;
^{d} p < 0.05 vs 75 kDa;
^{e} p< 0.001 vs cisplatin and HA-Pt;
^{f} p < 0.01;
N.T. (not tested);
N ≥ 5.

### Example 17

### Pt-DNA Adduct and Plasma Pt Quantitation

Female Sprague-Dawley rats (225-275 g; Charles River, Wilmington, Mass.) were treated with cisplatin (10 mg/kg, i.v. tail vein) or 75HA-Lys-Pt (10 mg/kg cisplatin basis, s.c. cheek directly below ear) under 4% isoflurane (VetOne, Boise, Idaho) anesthesia. After 15 minutes to 96 hours, whole blood was collected via cardiac puncture into heparin collection tubes. Whole blood was centrifuged (1500×g, 5 min., 4° C., no brake). Top layer (plasma) and middle layer (white blood cells) were transferred to new tubes. Plasma was frozen (−20° C.) until analysis. Ice-cold PBS (137-mM NaCl, 2.7-mM KCl, 10-mM Na₂HPO₄, 1.8-mM KH₂PO₄, pH 7.4, 5 mL) and red blood cell lysis buffer (0.83% NH₄Cl, 0.1% KHCO₃, 1-mM EDTA, 15 mL) were added to white blood cells, shaken gently, incubated on ice (15 min), and centrifuged (1500×g, 5 min., 4° C.). The white blood cell pellet was washed twice with cold PBS and re-suspended in 9 mL of white blood cell buffer (10-mM Tris, pH 7.4, 2.3% NaCl, 2-mM EDTA), and 900 μL of 1.1-M NH₄HCO₃, 900 μL of 10% SDS, and 75 μL of 2% Proteinase K were added and incubated at 42° C. overnight. Then, 3.3 mL of 5-M NaCl was added, samples shaken vigorously, and centrifuged twice (3200×g, 5 min, 4° C.), each time discarding the pellet. DNA was pelleted with 20 mL of ice-cold 100% ethanol and centrifuged (3200×g, 5 min, 4° C.); the DNA pellet was washed twice with 75% ethanol, re-suspended in water, and quantitated. DNA was digested in 1% HNO₃ (800 μL total volume) overnight at 70° C. DNA samples were diluted 1:10 in 1% HNO₃ and plasma samples were diluted 1:10,000 in 1% HNO₃. Diluted samples were analyzed for Pt content using ICP-MS with a calibration curve from 1-1000 ppt.

The formation of DNA-platinum adduct contributes to the cytotoxicity of cisplatin and DNA-platinum adduct formation in white blood cells are often used as a proxy for adduct formation in tumors. Therefore, we compared DNA-platinum adduct formation in PBMCs of healthy rats over time between cisplatin and 75HA-Lys-Pt ((A) of FIG. 10). Cisplatin shows consistent levels of adduct formation (11-16 pg Pt/μg DNA) over the time course with an area-under-the-curve (AUC) of 1466 pg Pt×h/μg DNA). In contrast, HA-Lys-Pt shows increasing adduct formation over time peaking at 48 hours (106 pg Pt/μg DNA), followed by a reduction over the next 2 days. The resulting AUC for 75HA-Lys-Pt is 4730 pg Pt×h/μg DNA. Simultaneously, we measured Pt content in plasma from the same animals ((B) of FIG. 10). Cisplatin shows an early spike in plasma Pt concentration at 15 minutes of 7 μg Pt/mL plasma with an AUC of 102.2 pg Pt×h/mL. In contrast to cisplatin, but similar to adduct formation, 75HA-Lys-Pt shows increasing plasma Pt levels over time plateauing between 24 and 72 hours with concentration between 4-5 μg Pt/mL plasma with an AUC of 398.5 pg Pt×h/mL.

### Example 18

### Toxicity in Mice and Rats

The toxicity of HA-Lys-Pt was evaluated in healthy Balb/c mice. A group of Balb/c mice was injected subcutaneously with 20 (N=2), 30 (N=3), and 40 mg/kg (N=2) of the 75HA-Lys-Pt. Another group of Balb/c mice was injected subcutaneously with 8.86 (N=2), 13.3 (N=2), 17.7 (N=2), 20 (N=2), 30 (N=2), and 40 mg/kg (N=1) of the 33HA-Lys-Pt. Their body weights and symptoms of toxicities were monitored twice per week for up to 2 months.

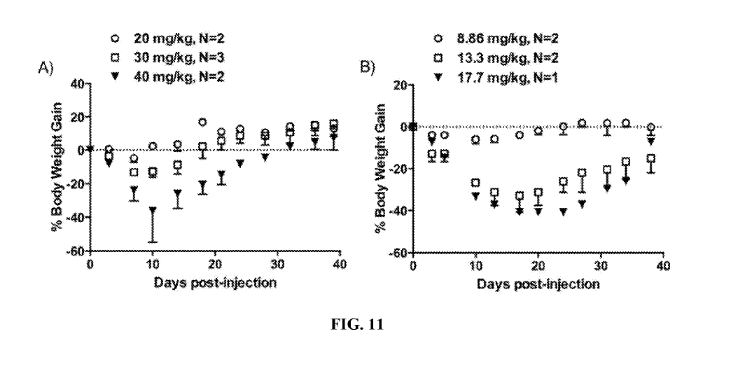

To assess the tolerability of HA-Lys-Pt, healthy Balb/C mice were given six different doses of the drug, and their body weights and toxic reactions were monitored twice per week for 40 days (FIG. 11). All mice maintained normal body conditions and had some degree of weight loss during the treatment, but returned to pre-dose weights at the completion of the study. Mice that received a dose of 20 or 30 mg/kg (on cisplatin basis) began to lose weight one-week post treatment but returned to pre-dose weight within another two weeks. It took one additional week for the mice that received the highest dose (40 mg/kg) to return to pre-dose weight. Acute toxic reactions including lethargy, paralysis, and respiratory depression were not observed at any dose levels during the entire study.

### Example 19

### Anticancer Efficacy in Mice

The MDA-1986 cells were prepared in PBS at a concentration of 2×10⁷ cells/mL. Female NU/NU mice were anesthetized under 2% isoflurane in O₂, and 50 μL of cell solution was injected into the oral sub-mucosa of the mice using a 27-ga needle. All procedures in the animal study were approved by the Institutional Animal Care and Use Committee of the University of Kansas. Tumor growth was monitored twice weekly by measurement with a digital caliper, and the tumor volume was calculated as 0.52×(width)²×(length). The primary cheek tumors reached 50-100 mm³ typically after 2 weeks, and HA-Lys-Pt was administered peritumorally subcutaneously at 20 mg/kg on the basis of cisplatin. Cisplatin was administered intravenously via tail veins at a dose of 10 mg/kg, the maximum determined previously. The drugs were given weekly for 3 consecutive weeks.

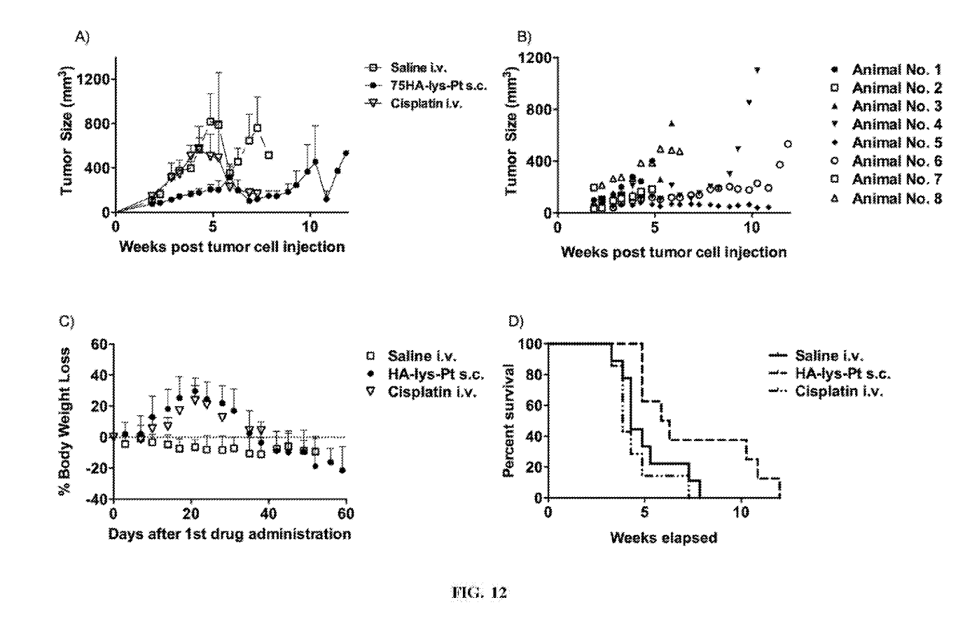

An orthotropic xenograft model of human HNSCC was established to examine the anti-cancer efficacy of HA-Lys-Pt. Tumors on mice in the control group reached a size of approximately 1000 mm³ within 6 weeks after tumor cell implantation. In comparison, the average tumor growth of mice treated with 75HA-Lys-Pt began to show a lower rate of proliferation immediately after the first treatment at the beginning of the third week. Tumor progression was delayed by four weeks after subcutaneous 75HA-Lys-Pt therapy (student t test, p<0.05). The treated animals developed tumors of an average size less than 200 mm³ by the end of the sixth week ((A) of FIG. 12), and the survival rate was significantly extended relative to the control and cisplatin treated groups ((D) of FIG. 12); Log-rank (Mantel-Cox) test, p<0.05). A single dose of 20 mg/kg HA-Lys-Pt resulted in less than 5% of weight loss, whereas three weekly doses of 20 mg/kg caused more than 20% weight loss after the last injection, though body weight returned to normal three weeks after the last injection ((C) of FIG. 12).

The treatment outcome of each individual animal is reported in Table 2 and (B) of FIG. 12. The rates of partial response (PR) and stable disease (SD) were 62.5% and 37.5% for 75HA-Lys-Pt treated animals and 75% and 25% for 33HA-Lys-Pt treated animals, respectively. Neither group contained any animals with progressive disease (PD). However, the rates of PR, SD, and PD were 37.5%, 12.5%, and 50% for cisplatin treated groups, which suggests significantly lower effectiveness of the standard-of-care IV therapy. As a control arm, 100% of the saline treated animals developed PD shortly after tumor implantation.

Table 2 shows tumor response based on Modified RECIST Criteria. 75HA-Lys-Pt stands for HA-Lys-Pt made with 75 kDa HA. 22HA-Lys-Pt stands for HA-Lys-Pt made with 33 kDa HA. PR=Partial Response (>30% reduction); PD=Progressive Disease (>30% tumor growth); SD=Stable Disease (neither PR nor PD criteria met).

TABLE 2

| Treatment | #1 | #2 | #3 | #4 | #5 | #6 | #7 | #8 |
|---|---|---|---|---|---|---|---|---|
| 75HA-Lys-Pt | PR | PR | PR | SD | SD | PR | SD | PR |
| 33HA-Lys-Pt | PR | PR | SD | PR | PR | SD | PR | PR |
| Cisplatin | PR | PD | SD | PR | PD | PR | PD | PD |
| Saline | PD | PD | PD | PD | PD | PD | PD | PD |

### Example 20

### Pharmacokinetics in a Healthy Dog

Pharmacokinetics of HA-Lys-Pt were determined in a healthy dog by Xenometrics LLC (Stillwell, Kans.). A naive female dog (ca. 6 kg) was given 75HA-Lys-Pt into the oral sub-mucosa; 1.5 mg/kg on cisplatin basis on day 1 and 2.0 mg/kg on day 68. After each injection, 2-mL serum samples were collected at 0, 0.25, 1, 2, 6, 24, 48, 96, and 168 hours. Cumulative urine samples were collected at 0-6, 6-24, 24-48, 48-72, 72-96, 96-120, 120-144, and 144-168 hours. The samples were frozen at −80° C. until ICP-MS analysis. Prior to analysis, serum and urine samples were diluted 250 and 500-fold using 1% HNO₃, respectively. The platinum calibration curve contained 4 replicates of 6 concentrations (0, 1, 10, 20, 40, and 50 ppb). The internal standard was bismuth at 50 ppb.

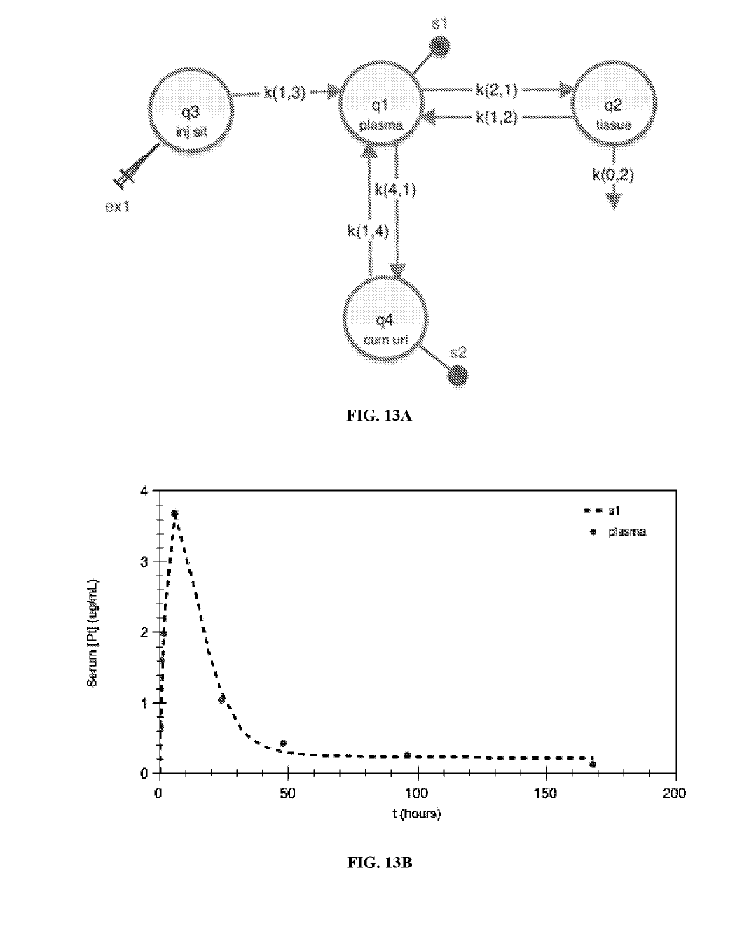

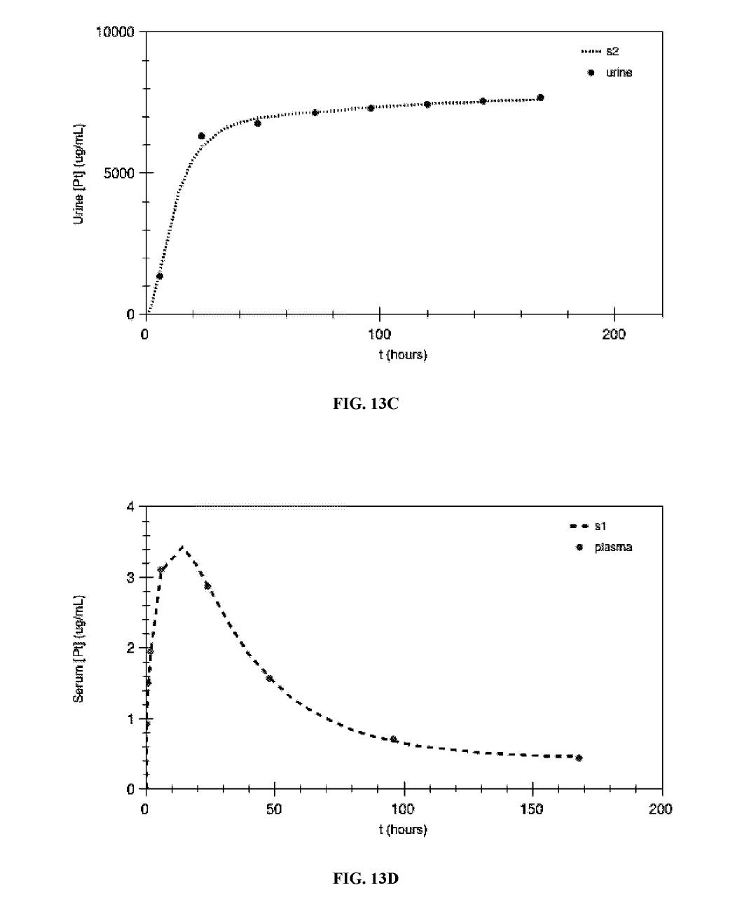

The pharmacokinetics of the HA-Lys-Pt was measured using the serum and urine samples. The data was modeled using SAAM II software with a four compartmental model. FIG. 13A demonstrates the model, and FIGS. 13B, 13C and 13D show the serum drug concentration vs. time curve and the cumulative drug concentration in urine vs. time curve, respectively, after the first and the second dose.

As indicated by the model, q1, q2, q3, and q4 represent the central blood compartment, the body/tissue compartment, the injection site, and the urine compartment, respectively. The syringe represents the dose that was given to the injection site compartment. The two red circles indicate the two sampling sources, the serum from the q1 (s1), and the urine from the q4 (s2). Furthermore, five transfer rate constants and one loss rate constant were applied to the model to link the compartments and their individual differential equations for data analysis and simulation. The assignment of rate constants and pharmacokinetic interpretation has been discussed previously. The calculated PK parameters including AUC, clearance, volume of distribution at steady state (Vₛₛ), elimination half-lives (tₑ), and transfer rate constants, are listed in Table 3.

Table 3 shows the pharmacokinetic parameters calculated from a four-compartmental model using SAAM II software. *Pharmacokinetics of HA-Pt treated dogs were modeled using a three-compartmental model; thus, the transfer rate constants were not compared. HA-Pt stands for a hyaluronan-cisplatin conjugate made by attaching cisplatin directly to HA.

TABLE 3

| | 75HA-Lys-Pt, | 75HA-Lys-Pt, | |
|---|---|---|---|
| Parameters | 1^{st} dose | 2^{nd} dose | HA-Pt* |
| Dose, mg/kg | 1.5 | 2.0 | 0.5-1.5 |
| Cₘₐₓ, μg/mL | 3.680 | 3.437 | 0.997 ± 0.956 |
| Tₘₐₓ, h | 6 | 14.4 | 0.5 or 1 |
| AUC, μg × h/mL | 102 | 218 | 6.3 ± 3.6 |
| Clearance, mL/h/kg | 17.0 | 9.2 | 43.5 ± 20.2 |
| Vₛₛ, L/kg | 7.4 | 2.5 | 2.1 ± 1.3 |
| tₑ (β), h | 6.1 | 3.1 | 3.8 ± 2.9 |

### Example 21

### Clinical Chemistry, Hematology, and Urinalysis in a Healthy Dog

Before each dose, the dog was examined by clinical chemistry, hematology, and urinalysis. Additional examinations of clinical chemistry and hematology were performed on days 7, 14, and 42 after the first dose. Additional tests of urinalysis were performed on days 7 and 14 after the first dose. The potential drug-induced toxicities were monitored for 56 days after the second dose. Specifically, clinical chemistry and urinalysis were performed on days 7, 14, 21, and 56, and hematology was performed on days 7, 14, and 56 after the second dose to evaluate the tolerability and toxicity of the formulation.

The clinical chemistry panel (Na, K, Cl, glucose, Ca, urea nitrogen, creatinine, total protein, albumin, total bilirubin, and inorganic phosphorus) was unaffected by dosing. Creatinine kinase, globulinm, and A/G ratio varied slightly between time points, but still remained in the normal ranges (REF column in Table 4). Alkaline phosphatase (ALP), alanine aminotransferase (ALT), and aspartate aminotransferase (AST) levels are commonly used markers for evaluating liver function after chemotherapy. Elevated levels of these liver enzymes usually indicate some degree of hepatic toxicity due to damage to the liver. Although slightly elevated, the ALP and ALT levels fell in the normal ranges after the first dose. AST was mildly elevated but returned to a near pre-dose value on the 42^{nd} day. In comparison, the second dose (2.0 mg/kg) resulted in more significant increases in the enzyme levels post-dose. The ALP, ALT, and AST elevated by 0.7-, 4.6-, and 8.7-fold, respectively, at 7-days post-dose. However, the increases were reversible, and at 14-days post-dose the ALT and AST fell to 2.8- and 2.7-fold of the pre-dose values. The ALP level returned to only 0.27-fold higher than the pre-dose level by 8-weeks post-dose. In summary, the first dose (1.5 mg/kg) resulted in slight reversible changes to the liver enzymes and it appeared to be tolerable. The second dose (2.0 mg/kg) triggered prolonged elevation of the liver enzymes; however, the elevations were reversible and levels began to recover starting at two weeks post injection. In addition, the serum cholesterol levels dropped after both injections, suggesting reduced liver biosynthesis capacity possibly due to the administration of the drug.

White and red blood cell counts, hemoglobin, hematocrit, and differential white blood cell count varied slightly over time, but the values were within the normal range (REF column in Table 5). The platelet counts were reduced after both injections; however, the reduction was greater after the second injection, suggesting that bone marrow toxicity may be dose-dependent. The bone marrow toxicity was reversible as the platelet counts increased from 36×10³/μL at 7-day to 232×10³/μL at 14-day after the second dose. This result suggests that a minimum of a two-week interval between doses is necessary for platelet counts to recover.

The urinalysis results within normal ranges except for the 14-day test result after the second dose (Tables 6 and 7), in which elevated levels of glucose and protein were detected in the urine samples, suggesting potential early signs of kidney damage. However, seven days later, the glucose became negative and the protein level was 30 mg/dL, which is lower than the pre-dose value of the first dose. This result suggests that the drug may affect the kidney temporarily and the effect is dose-dependent. Though cisplatin is known to cause nephrotoxicity, the 75HA-Lys-Pt did not appear to cause long-term renal damage to a healthy dog.

Table 4 shows the clinical chemistry of a dog received 1.5 mg/kg of 75HA-Lys-Pt on day 1 and 2.0 mg/kg of 75HA-Lys-Pt on day 70. Clinical chemistry examinations were performed at pre- and post-dose for both injections. The injections were given via the sub-mucosa in the mouth.

TABLE 4

| | 1.5 mg/kg (30 mg/m²) | | | | 2 mg/kg (40 mg/m²) | | | | | |
|---|---|---|---|---|---|---|---|---|---|---|
| | | 7 days | | | | 7 days | 14 days | 21 days | 56 days | |
| Parameter | Predose | after | 14 days | 42 days | Predose | after | after | after | after | REF |
| Na (mmol/L) | 145 | 148 | 149 | | 147 | 148 | 152 | 147 | 150 | |
| K (mmol/L) | 4.9 | 4.0 | 5.0 | | 4.4 | 3.8 | 4.5 | 4.4 | 4.0 | |
| Cl (mmol/L) | 114 | 112 | 119 | | 113 | 115 | 122 | 113 | 119 | |
| Glucose (mg/dL) | 83 | 70 | 80 | | 75 | 83 | 76 | 64 | 73 | |
| Calcium (mg/dL) | 9.2 | 8.8 | 9.4 | | 10.1 | 9.3 | 8.6 | 9.3 | 9.7 | |
| Urea Nitrogen (mg/dL) | 18 | 13 | 19 | | 18 | 11 | 14 | 14 | 14 | |
| Creatinine (mg/dL) | 0.4 | 0.33 | 0.35 | | 0.43 | 0.25 | 0.3 | 0.33 | 0.3 | |
| Creatine kinase (U/L) | 123 | 93 | 207 | 159 | 139 | 105 | 143 | 113 | 117 | 59-895 |
| Total Protein (g/dL) | 4.6 | 5.0 | 5.2 | 5.5 | 5.5 | 5.4 | 5.5 | 5.8 | 6.3 | 5-7.5 |
| Albumin (g/dL) | 2.9 | 2.4 | 2.4 | 3.0 | 3.0 | 2.5 | 2.4 | 2.7 | 3.0 | 2.7-4.4 |
| Globulin (g/dL) | 1.7 | 2.6 | 2.8 | 2.5 | 2.5 | 2.9 | 3.1 | 3.1 | 3.3 | 1.6-3.6 |
| A/G Ratio | 1.71 | 0.92 | 0.86 | 1.2 | 1.2 | 0.86 | 0.77 | 0.87 | 0.91 | 0.8-2 |
| Gamma glutamyltransferase (U/L) | — | — | — | — | 0 | 0 | 0 | 0 | 0 | |
| Alkaline phosphatase (U/L) | 97 | 132 | 116 | 125 | 136 | 237 | 249 | 219 | 173 | 10-150 |
| Alanine aminotransferase (U/L) | 29 | 36 | 57 | 74 | 59 | 330 | 223 | 198 | 220 | 5-107 |
| Aspartate aminotransferase (U/L) | 22 | 67 | 89 | 39 | 32 | 309 | 119 | 129 | 138 | 5-55 |
| Total Bilirubin (mg/dL) | 0.0 | 0.1 | 0.0 | 0.0 | 0.0 | 0.3 | 0.1 | 0.2 | 0.4 | 0-0.4 |
| Inorganic Phosphorus (mg/dL) | 4.1 | 3.3 | 5.5 | | 4.7 | 2.8 | 3.4 | 3.9 | 2.5 | |
| Cholesterol (mg/dL) | 127 | 89 | 97 | | 101 | 81 | 75 | 70 | 86 | |
| Triglyceride (mg/dL) | 26 | 34 | 38 | | 25 | 32 | 43 | 39 | 42 | |

Table 5 shows the hematology of a dog received 1.5 mg/kg of 75HA-Lys-Pt on day 1 and 2.0 mg/kg of 75HA-Lys-Pt on day 70. Hematology examinations were performed at pre- and post-dose for both injections. The injections were given via the sub-mucosa in the mouth.

TABLE 5

| | | 1.5 mg/kg (30 mg/m²) | | | | 2 mg/kg (40 mg/m²) | | | | |
|---|---|---|---|---|---|---|---|---|---|---|
| | | | 7 days | | | | 7 days | 14 days | 56 days | |
| Parameter | | Predose | after | 14 days | 42 days | Predose | after | after | after | REF |
| White blood cell count (10³/μL) | | 8.62 | 7.69 | 12.91 | 10.82 | 9.65 | 8.2 | 10.43 | 11.3 | 4.0-15.5 |
| Red blood cell count (10⁶/μL) | | 6.41 | 5.53 | 5.56 | 6.83 | 6.71 | 5.79 | 5.47 | 5.64 | 4.8-9.3 |
| Hemoglobin (g/dL) | | 14.7 | 12.7 | 12.6 | 15.3 | 14.8 | 12.7 | 11.8 | 12.1 | 12.1-20.3 |
| Hematocrit (%) | | 44.5 | 37.8 | 38.2 | 46.4 | 43.9 | 38 | 35.6 | 36.9 | 37-55 |
| Mean corpuscular (cell) volume (fL) | | 69.5 | 68.4 | 68.7 | 67.9 | 65.4 | 65.6 | 65.2 | 65.4 | 58-79 |
| Mean corpuscular (cell) hemoglobin (pg) | | 22.9 | 23.0 | 22.6 | 22.4 | 22.1 | 21.9 | 21.6 | 21.4 | 19-28 |
| Mean corpuscular (cell) hemoglobin | | 32.9 | 33.6 | 33.0 | 33.0 | 33.7 | 33.4 | 33.1 | 32.8 | 30-38 |
| concentration (g/dL) | | | | | | | | | | |
| Red cell distribution width (%) | | 13.9 | 13.3 | 13.7 | 12.4 | 13.1 | 13.5 | 14.2 | 13.7 | 14-19 |
| Hemoglobin distribution width (g/dL) | | 1.97 | 2.08 | 2.29 | 1.71 | 1.68 | 1.84 | 2.33 | 2.04 | NA |
| Platelet count (10³/μL) | | 411 | —^{a} | 307 | 307 | 344 | 36 | 232 | —^{b} | 170-400 |
| Differential white blood cell | % NEUT | 50.9 | 55.4 | 55.4 | 53.1 | 58.4 | 60 | 62.7 | 63.4 | NA |
| count (%) | % LYMPH | 39.5 | 37.6 | 37.6 | 37.6 | 32.9 | 29 | 28.5 | 26.6 | NA |
| | % MONO | 4.3 | 3.8 | 3.8 | 5.1 | 4.4 | 7.5 | 4.3 | 4.5 | NA |
| | % EOS | 3.7 | 2.0 | 2.0 | 2.0 | 2.3 | 1.9 | 3.4 | 4.3 | NA |
| | % BASO | 0.9 | 0.7 | 0.7 | 1.4 | 1.2 | 0.9 | 0.5 | 0.7 | NA |
| | % LUC | 0.6 | 0.6 | 0.6 | 0.8 | 0.8 | 0.8 | 0.6 | 0.5 | NA |

| | | | | | | | | | |
|---|---|---|---|---|---|---|---|---|---|
| Reticulocyte count (%) | 1.67 | 1.40 | 1.84 | 0.91 | 1.83 | 1.59 | 3.2 | 1.3 | 0-1.5 |

^{a}Platelets appear adequate. Platelet clumps observed, number not reported.
^{b}not available due to blood clots

Table 6 shows the urinalysis of a dog received 1.5 mg/kg of 75HA-Lys-Pt on day 1 and 2.0 mg/kg of 75HA-Lys-Pt on day 70. Urianlyses were performed at pre- and post-dose for both injections. The injections were given via the sub-mucosa in the mouth.

TABLE 6

| | 1.5 mg/kg (30 mg/m²) | | | 2 mg/kg (40 mg/m²) | | | | |
|---|---|---|---|---|---|---|---|---|
| Parameter | Predose | 7 days after | 14 days | Predose | 7 days after | 14 days after | 21 days after | 56 days after |
| Glucose (mg/dL) | Negative | Negative | Negative | Negative | Negative | 100 | Negative | Negative |
| Bilirubin | Small | Negative | ç | Small | Moderate | Negative | Moderate | Moderate |
| Ketones (mg/dL) | Negative | Negative | Negative | Negative | Negative | Negative | Trace | Negative |
| Blood | Negative | Negative | Large | Negative | Negative | Large | Small | Large |
| pH | 7.0 | 8.0 | 6.5 | 6.0 | 7.0 | 7.5 | 7.0 | 8.0 |
| Protein (mg/dL) | 100 | Negative | 30 | Trace | 30 | ≥300 | 30 | 30 |
| Urobilinogen (E.U./dL) | 1.0 | 0.2 | 0.2 | 0.2 | 0.2 | 0.2 | 1.0 | 1.0 |
| Nitrite | Negative | Negative | Negative | Negative | Negative | Negative | Negative | Negative |
| Leukocytes | Negative | Negative | Negative | Negative | Negative | Negative | Negative | Negative |
| Color | Dark Yellow | Yellow | Yellow | Dark Yellow | Dark Yellow | Light Yellow | Dark Orange | Dark Orange |
| Clarity | Clear | Cloudy | Cloudy | Clear | Clear | Clear | Clear | Clear |
| Specific Gravity | 1.060 | 1.023 | 1.038 | 1.051 | 1.053 | 1.010 | 1.044 | 1.030 |

Table 7 shows the microscopic urinalysis of a dog received 1.5 mg/kg of 75HA-Lys-Pt on day 1 and 2.0 mg/kg of 75HA-Lys-Pt on day 70. Microscopic urinalyses were performed at pre- and post-dose for both injections. The injections were given via the sub-mucosa in the mouth.

TABLE 7

| | 1.5 mg/kg (30 mg/m²) | | | 2 mg/kg (40 mg/m²) | | | | |
|---|---|---|---|---|---|---|---|---|
| Parameter | Predose | 7 days after | 14 days | Predose | 7 days after | 14 days after | 21 days after | 56 days after |
| Red Blood Cells | — | — | — | — | — | 16-30 | 1-5 | TNTC |
| White Blood Cells | — | — | — | — | — | — | — | — |
| Cast | — | — | — | — | Rare triple | — | — | — |
| | | | | | phosphate | | | |
| Epithelial | — | — | — | — | — | — | — | — |
| Mucus | — | — | — | — | — | — | — | — |
| Bac-U | Few | Moderate | Few | Rare | — | — | Few | Rare |
| Sperm-U | — | — | — | — | — | — | — | — |
| Trpl Ph Crystal | Moderate | Few | — | Rare triple | Rare | — | Rare triple | Few |
| | | | | phosphate | | | phosphate | |

### Example 22

### Pharmacokinetics in Three Tumor-Bearing Dogs

An open label, multiple dose/dosage phase I/II trial of 75HA-Lys-Pt was conducted at the University of Missouri Veterinary Medical Teaching Hospital (MU-VMTH). The clinical trial protocol was approved by the University of Missouri ACUC. Dogs of any age, sex, or breed presented to the MU-VMTH oncology service with the a histological diagnosis of anal sac carcinoma, oral squamous cell carcinoma, oral melanoma, nasal carcinoma, or digital squamous cell carcinoma with a performance score of 0 or 1 (0: normal activity; 1: restricted activity; 2: compromised activity; 3: disabled; 4: dead) and without comorbid conditions that would limit life expectancy to less than nine weeks were offered enrollment in the trial.

Eligible dogs had to meet the following criteria: a tumor>2 cm in longest dimension; fully staged with CBC, chemistry profile, thoracic radiographs (SCC of mouth or forepaw and melanoma), abdominal ultrasound (SCC of hindpaw or anal sac carcinoma), and fine needle aspiration and cytology of the draining lymph node. Dogs must not have had NSAIDs (nonsteroidal anti-inflammatory drugs) administered within seven days of starting the trial or during the trial. In addition, dogs were excluded from eligibility if they had received immunotherapy, radiation therapy, or another chemotherapy within 3 weeks of the trial, had grade 1 or higher renal dysfunction, persistent neutropenia or thrombocytopenia, or a significant infection of their tumor that could not be readily managed.

Following complete staging, client consent was obtained to enroll each dog in the study. Once enrolled in the study, longest tumor diameters of all target lesions were recorded. An indwelling jugular catheter was placed to facilitate collection of blood samples. Dogs received 10 to 30 mg/m² intra-tumoral injections through 1 to 3 pre-placed 22-ga spinal needles once every three weeks for up to four planned doses. Dogs were sedated or anesthetized for each treatment, and a 2-mL blood sample was collected from the jugular catheter at 0.5, 1, 2, 4, and 24 hours following drug administration. Blood samples were spun, plasma was collected and frozen within two hours. A CBC and renal profile with urinalysis were conducted prior to and one week after each treatment. Some dogs experiencing apparent adverse effects of drug administration had complete biomedical profiles performed. Tumor measurements were collected three weeks following each administration to assess response.

Dogs were withdrawn from the study if they experienced grade 3 nephrotoxicity, grade 3 local reaction, progression of disease while on study, or client request to withdraw from study. Necropsy was requested from all dogs dying during or after the study period to identify tumor response and evidence of systemic toxicity.

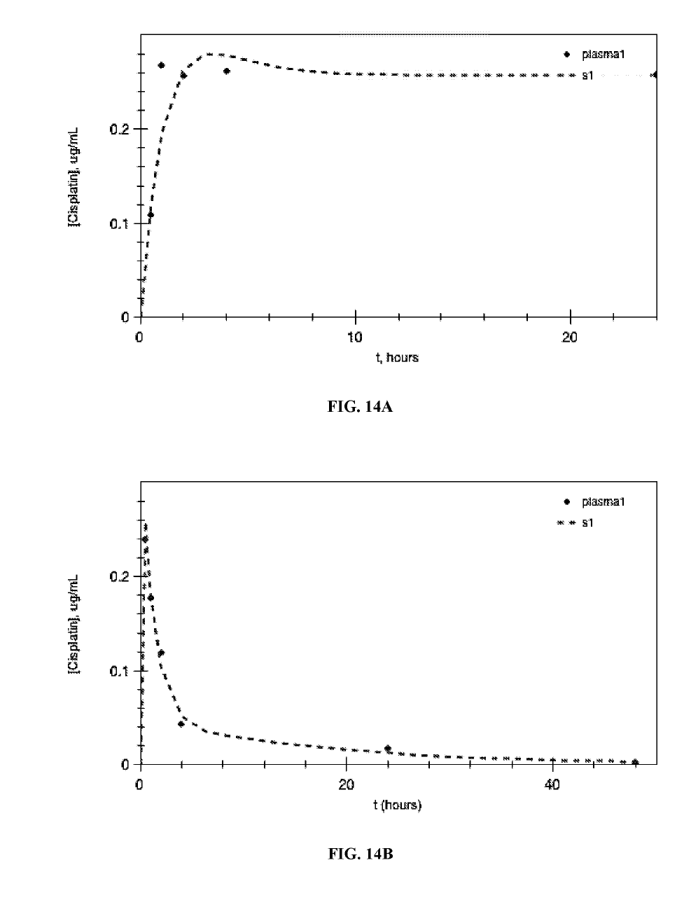

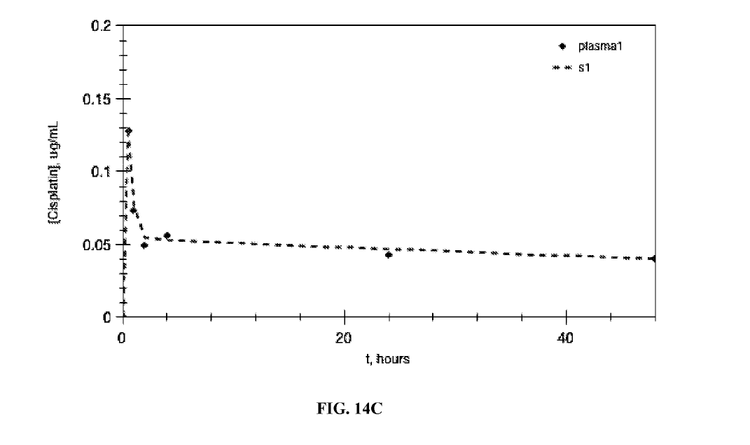

Serum samples of 3 dogs were diluted 20-fold in 1% HNO₃, and vortexed prior to inductively coupled plasma mass spectrometry (ICP-MS) analysis. The platinum concentration of serum was determined using an Agilent 7500i ICP-MS. A calibration curve was generated from 1 to 50 ppb platinum (acceptable criterion R²>0.995) and 50-ppb bismuth was used as an internal standard. Unknown samples were interweaved with a 20-ppb platinum quality control; the acceptance criterion was an IS recovery of 80-120%. The pharmacokinetics were modeled using a SAAM II three-compartmental model. The results are shown in FIGS. 14A, 14B, and 14C.

The serum pharmacokinetics data was modeled using a three-compartmental model with an injection site compartment (q3), a serum compartment (q1), and a tissue compartment (q2). The syringe represents the bolus dosing of 75HA-Lys-Pt (ex1), which is associated with the injection site compartment, while the dot (s1) represents the sampling event associated with the serum compartment. Three transfer constants, k(1,3), k(1,2) and k(2,1), and one loss constant, k(0,1), were assigned to the model. The k(1,3) defined the drug absorption process allowing the transfer of HA-Pt from the injection site compartment to the serum compartment. A reversible k(3,1) was not necessary as a sink condition was assumed. Due to the concentration gradient between the injection site and the central serum compartments, drug redistribution back to the injection site was not expected. A loss constant was designated by k(0,1) which represented the renal clearance of either the unchanged cisplatin or its metabolites. Drug transfer and equilibrium between plasma and tissue compartments were represented by k(1,2) and k(2,1). Post-injection drug diffused from the tissue at the injection site to the blood capillaries and the lymphatic vessels. Eventually the drug molecules entered the systemic circulation and distributed into organs. It is likely that the highly perfused organs, such as liver and kidneys, established equilibrium sooner than organs with low blood flow. The pharmacokinetic parameters were calculated and reported in Table 8.

Table 8 shows pharmacokinetic parameters of three dog received 10 mg/m² of 75HA-Lys-Pt. The injections were given via the sub-mucosa in the mouth.

TABLE 8

| | Treatment | | |
|---|---|---|---|
| | 75HA-Lys-Pt | | |
| Dog ID | 1 | 2 | 3 |
| Dose, mg/m2 | 10 | 10 | 10 |
| Weight, kg | 28.7 | 25.2 | 6.5 |
| AUC, ug × h/mL | 4 | 0.9 | 2.2 |
| Cₘₐₓ, ug/mL | 0.174 | 0.155 | 0.128 |
| Tₘₐₓ, h | 1 | 0.5 | 0.5 |
| Cle, mL/h/kg | 0.13 | 1.78175E−05 | 92 |
| Vₛₛ, L/kg | 2 | 33.4 | 8.9 |
| tₑ, h | 6076 | 69300000 | 11.1 |

### Example 23

### Synthesis of N_{α}-Acetyl-L-Glutamic acid α-tert-butyl ester (1)

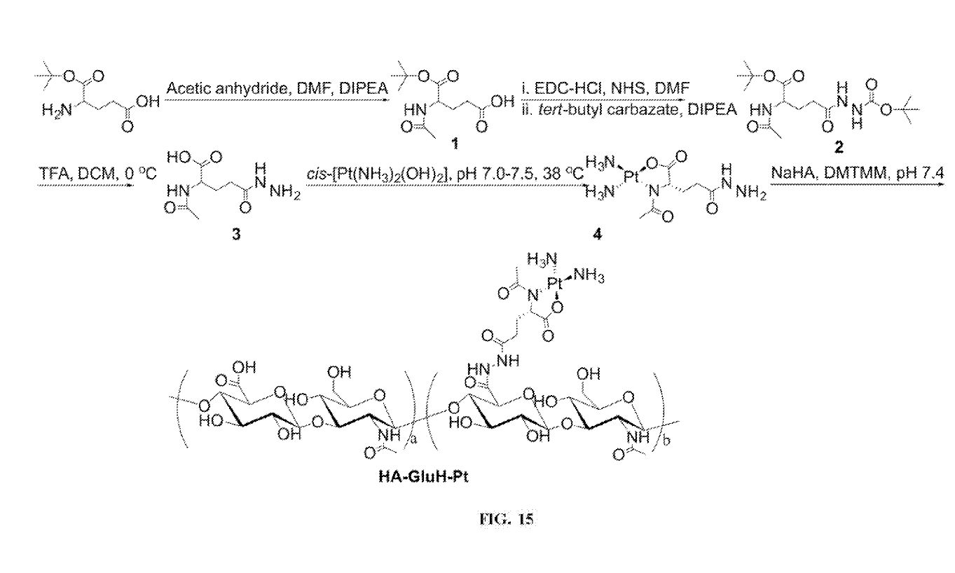

L-Glutamic acid α-tert-butyl ester (500 mg, 2.46 mmol) was added to a flame-dried flask equipped with a magnetic stir bar, and suspended in a mixture of 5-mL of anhydrous dimethylformamide (DMF) and 640-4, of N,N-diisopropylethylamine (DIPEA, 3.7 mmol). Acetic anhydride (280 μl, 3 mmol) was added dropwise, and the reaction was allowed to proceed at room temperature for 24 hours. After solvent was removed under reduced pressure, the desired compound was obtained without further purification and used directly in the next step. MS (ESI), calculated for C₁₁H₁₉NO₅ (M+Na)⁻: 268.1; found 268.1. The synthetic scheme for Examples 23-27 is shown in FIG. 15.

### Example 24

### Synthesis of tert-butyl 2-(4-acetamido-5-(tert-butoxy)-5-oxopentanoyl) hydrazinecarboxylate (2)

To a solution of compound 1 in 8-mL of anhydrous DMF, 566 mg of 1-ethyl-3-(3-dimethylaminopropyl)carbodiimide hydrochloride (EDC.HCl, 2.95 mmol) and 340 mg of N-hydroxysuccinimide (NHS, 2.95 mmol) were added. The mixture was stirred under argon at room temperature. After 3 hours, 487 mg of tert-butyl carbazate (2.95 mmol) was added to the mixture with 643 μL of DIPEA, and then stirred for another 20 hours. The solution was dried under reduced pressure and suspended in citric acid solution (25 mM, pH 4.0), followed by the extraction with ethyl acetate (EtOAc). The combined organic layers were washed with water and brine, dried over Na₂SO₄ and concentrated under reduced pressure. The crude product was chromatographed over silica gel (10:1 hexane/EtOAc, then 3:1, and finally with 1:2 hexane and EtOAc) to provide a white solid. ¹H-NMR (400 MHz, CDCl₃) δ (ppm): 8.70 (s, 1H), 4.54 (m, 1H), 2.31 (t, J=7.2 Hz, 2H), 2.07 (m, 2H), 2.03 (s, 3H), 1.89 (s, 18H). MS (ESI), calculated for C₁₆H₂₉N₃O₆ (M+H)⁻/(M+Na)⁺: 360.2/382.2; found 360.2/382.2.

### Example 25

### Synthesis of 2-acetamido-5-hydrazinyl-5-oxopentanoic acid (N_{α}-AcetylGluH, 3)

Compound 2 was dissolved in 1-mL of dichloromethane (DCM). One milliliter of trifluoroacetic acid (TFA) was added slowly at 0° C. in an ice bath. The solution was stirred at 0° C. for 3 hour and then room temperature for 3 hours. The excess TFA was removed under reduced pressure and the remaining residue was dissolved in water, and washed with EtOAc. The aqueous layer was collected and the water was removed by lyophilization to afford 320 mg of the desired compound as a white solid. Yield: 64%. ¹H-NMR (400 MHz, MeOD) δ (ppm): 4.24 (m, 1H), 2.19 (t, J=7.2 Hz, 2H), 2.03 (m, 2H), 1.96 (s, 3H). MS (ESI), calculated for C₇H₁₃N₃O₄ (M+H)⁺/(M+Na)⁺: 204.09/226.08; found 204.10/226.08.

### Example 26

### Synthesis of cis-[Pt(NH₃)₂(N_{α}-AcetylGluH)] (4)

Cisplatin (60 mg, 200 μmol) was added to 3-mL of warm water. The resulting suspension was added AgNO₃ (68 mg, 400 μmol) and allowed to stir for 1 day at room temperature in the dark. After AgCl₂ was removed by centrifuge at 4000 rpm for 10 min, the supernatant was further filtered by syringe filter (pore size 0.22-μm) to obtain cis-[Pt(NH₃)₂(OH)₂]. N_{α}-AcetylGluH (40 mg, 200 μmol) was added, and the pH of the mixture was adjusted above 5.0 using NaOH (1 N). After stirring at room temperature for overnight, the pH dropped to ˜4.0 and then was adjusted to 7.0-7.5. The reaction mixture was heated up to 38° C. and incubated at 38° C. for another 3 days in the dark. The pH was monitored during the reaction and kept at 7.0-7.5. The resulting solution was concentrated under reduced pressure. ¹³C-NMR (500 MHz, D₂O) δ (ppm): 189.4, 184.1, 174.2, 64.5, 29.3, 26.7, 21.9.

### Example 27

### Synthesis of HA-cis-[Pt(NH₃)₂(N_{α}-AcetylGluH)] (HA-GluH-Pt) Conjugates

Seventy-nine milligrams of sodium hyaluronate (NaHA, 35 KDa) were dissolved in 1-mL of sodium phosphate buffer (1 mM, pH 7.4), to which a 4-(4,6-dimethoxy-1,3,5-triazin-2-yl)-4-methylmorpholinium chloride (DMTMM, 109 mg, 0.39 mmol) solution in sodium phosphate buffer was added. To this mixture, 260-4, of cis-[Pt(NH₃)₂(N_{α}-AcetylGluH)] (0.39 mmol) solution in water was added. The reaction mixture was adjusted pH to 7.4 using 0.1 N NaOH, and incubated without agitation at room temperature for 4 days in the dark. The resulting solution was filtered through a nylon syringe filter (pore size 0.22-μm) and transferred to dialysis tubing with 10,000 MWCO. Final product was purified by dialysis against water 4 hours, sodium phosphate solution (100 mM NaCl, 10 mM NaH2PO4, pH 7.4) 2 hours, and two water changes over another 18 hours. The loading degree of Pt was determined by inductively coupled plasma mass spectrometry (ICP-MS) to be 26% on cisplatin weight basis. The synthetic scheme is shown in FIG. 15. C-NMR (500 MHz, D₂O) δ (ppm): 189.4, 184.1, 178.1, 174.8, 174.6, 174.1, 103.1, 102.9, 82.9, 79.2, 76.2, 75.3, 74.0, 71.7, 68.4, 64.5, 60.5, 54.2, 29.3, 26.7, 21.9, 21.8.

### Example 28

### Cytotoxicity of HA-GluH-Pt

Cell growth inhibition was determined in 96-well plates using head and neck squamous cell carcinoma cells, MDA-1986. The IC50 was calculated to be 623 μM. Plates were seeded with 3,000 cells/well in 90 uL of media (6 replicates/sample). Ten microliters of HA-GluH-Pt conjugate solutions were applied after 24 hours. Resazurin blue in 10 uL of PBS was applied to each well (final concentration 5 uM) after another 72 hours. After 4 hours, the well fluorescence was measured (ex/em 560/590) (SpectraMax Gemini, Molecular Devices), and the IC₅₀ concentration was determined as the midpoint between drug-free medium (positive) and cell-free (negative) controls.

### Example 29

### Release of Platinum from HA-GluH-Pt

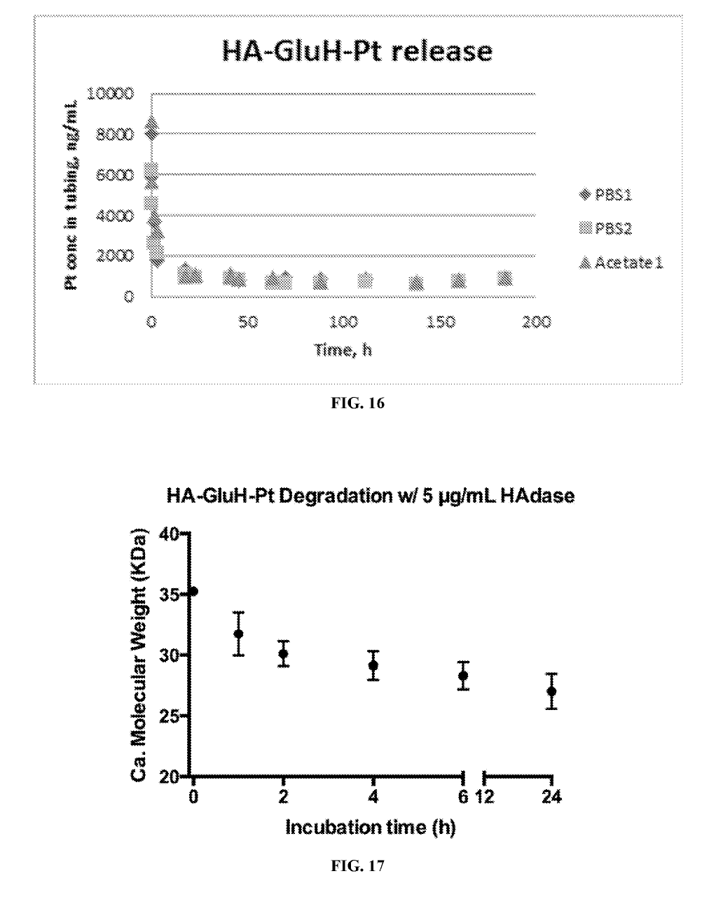

Release studies were conducted in phosphate buffered saline (PBS, 12 mM sodium phosphate, 137 mM NaCl, pH 7.4) and acetate buffered saline (12 mM sodium acetate, 137 mM NaCl, pH 5.5) at 37° C. A 0.1 mg/mL HA-GluH-Pt solution (concentration on cisplatin basis) was added into dialysis tubing with 10 kD MWCO. Dialysis tubing was placed in the buffer. At pre-determined intervals 100 μL of samples were collected and kept in −20° C. until analysis. Samples were diluted 1,000 fold using 1% HNO₃ prior to ICP-MS analysis. The platinum concentration in tubing was plotted in FIG. 16. The release half-lives of HA-GluH-Pt were less than 2 hours in both PBS and acetate buffers.

### Example 30

### Hyaluronidase Degradability of HA-GluH-Pt

The hyaluronidase (HAdase) specificity of HA-GLuH-Pt was evaluated by incubating 1.5 mg/mL HA-GluH-Pt (concentration on HA basis) with 5 μg/mL HAdase in sodium phosphate buffer containing 140 mM NaCl, 16 mM NaH₂PO₄, 7 mM Na₂HPO₄ at pH 6.4 at 37° C. for up to 24 hours. One hundred microliters of digested samples were collected at 0, 1, 2, 4, 6, and 24 hours and stored at −80° C. freezer until analysis. Samples (N=3) were diluted 5 fold using mobile phase at the time of analysis by size exclusion chromatography (SEC) with a Shodex OHpak SB-804 HQ column, coupled with a refractive index (RI) detector. The mobile phase was 5 mM ammonium acetate buffer (pH 5.0). The flow rate was 0.8 mL/min. The column temperature was 40° C. The injection volume was 80 μL. The HA-GluH-Pt degradation curve is shown in FIG. 17. HA-GluH-Pt maintained HAdase specificity and degradability. The molecular weight was reduced from 35 kD at t=0 h to 27 kD at t=24 h.

### Example 31

### CD44 Specificity of HA-GluH-Pt

CD44 specificity and cellular internalization mechanism of HA-GluH-Pt was investigated using MDA-1986 cells that overexpress CD44. Chlorpromazine was used as an endocytosis inhibitor. Cells were pretreated with 25-μM chlorpromazine or 10-μg/mL anti-CD44 1 h before the addition of 30-μg/mL HA-GluH-Pt (on platinum basis) and subsequent incubation for 6 h. Cells were trypsinized, lysed by sonication and diluted 20 fold prior to ICP-MS analysis. The result is shown in FIG. 18. The anti-CD44 antibody and chlorpromazine treated cells did not differ significantly from the non-treated control.

### Example 32

### Toxicity of HA-GluH-Pt in Mice

The toxicity of HA-GluH-Pt was evaluated in blab/C mice. Four blab/C mice were injected subcutaneously with 20, 40, 67 and 100 mg/kg HA-GluH-Pt. The body weights of the animals were checked weekly for 2 months. Less than 5% of weight loss was observed in all mice.

### Example 33

### Tumor Efficacy of HA-GluH-Pt in Mice

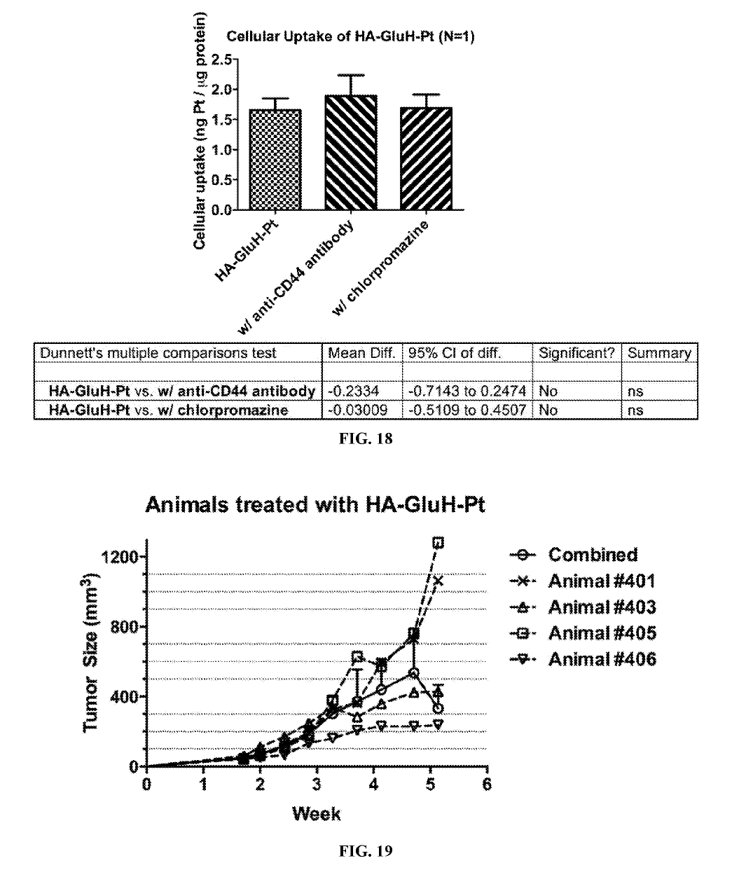

The MDA-1986 human head and neck squamous cell carcinoma cells were prepared in PBS at a concentration of 2×10⁷ cells/ml. Female NU/NU mice were anesthetized with 2% isoflurane in O₂, and 50 μL of cell solution was injected into the oral sub-mucosa of the mice using a 27-ga needle. All procedures in the animal study were approved by the Institutional Animal Care and Use Committee of the University of Kansas. The primary tumors with sizes between 50 to 150 mm³ were observed on the cheeks within 2 weeks after cell implantation. The tumor growth was monitored twice weekly by measurement with a digital caliper in two perpendicular dimensions, and the tumor volume was calculated using the equation: tumor volume=0.52×(width)²×(length). When the tumor reaches 50-100 mm³ in size, HA-GluH-Pt was administered subcutaneously next to the tumor at a dose of 20 mg/kg on the basis of cisplatin. The HA-GluH-Pt was given weekly for 5 weeks. The tumor response is shown in FIG. 19.

### Example 34

### HA-Lys-Pt Viscosity Reduction Using Mannitol

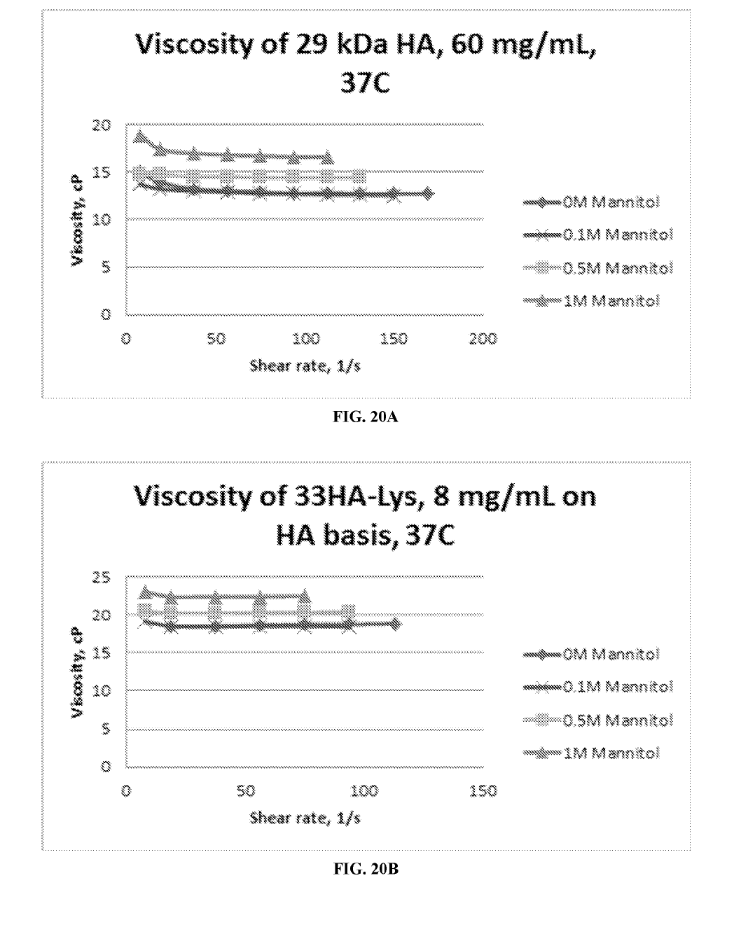

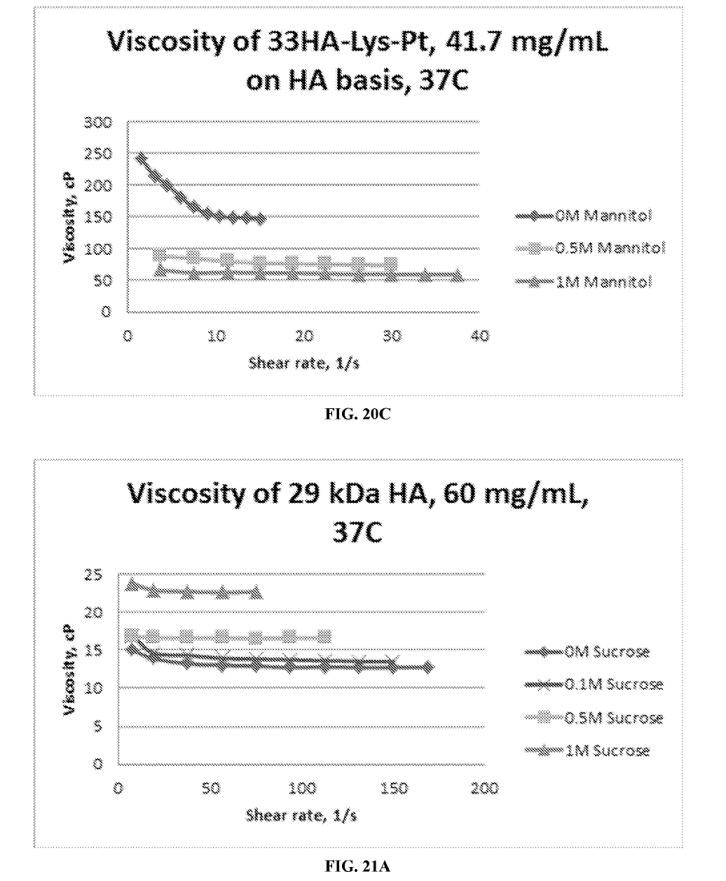

The viscosity of 29 kDa HA (60 mg/mL), 33HA-Lys (33 kDa HA with linker, 8 mg/mL on HA basis), and 33HA-Lys-Pt (41.7 mg/mL on HA basis) with increasing concentration of mannitol (0, 0.1, 0.5 and 1 M) were measured using a rheometer at 37° C. Mannitol (0.1, 0.5 and 1M) increased the viscosity of plain HA and 33HA-Lys, however, it unexpectedly reduced the viscosity of 33HA-Lys-Pt (FIGS. 20A, 20B, and 20C).

### Example 35

### HA-Lys-Pt Viscosity Reduction Using Sucrose

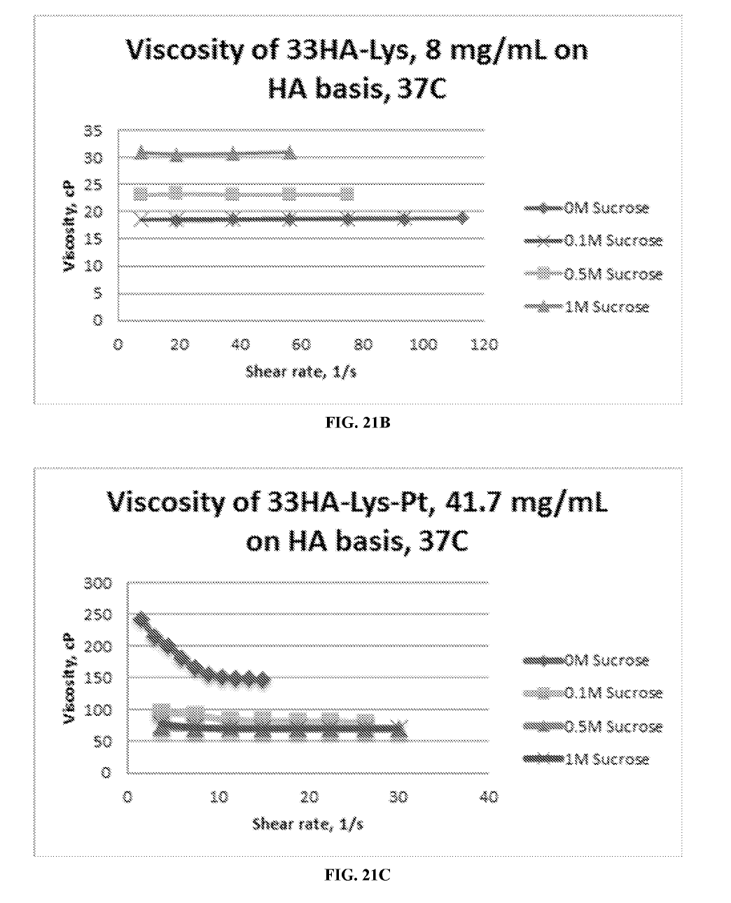

The viscosity of 29 kDa HA (60 mg/mL), 33HA-Lys (33 kDa HA with linker, 8 mg/mL on HA basis), and 33HA-Lys-Pt (41.7 mg/mL on HA basis) with increasing concentration of sucrose (0, 0.1, 0.5 and 1 M) were measured using a rheometer at 37° C. Sucrose (0.1, 0.5 and 1M) increased the viscosity of plain HA and 33HA-Lys, however, it unexpectedly reduced the viscosity of 33HA-Lys-Pt (FIGS. 21A, 21B, and 21C).

### Example 36

### HA-Lys-Pt Viscosity Reduction Using Trehalose

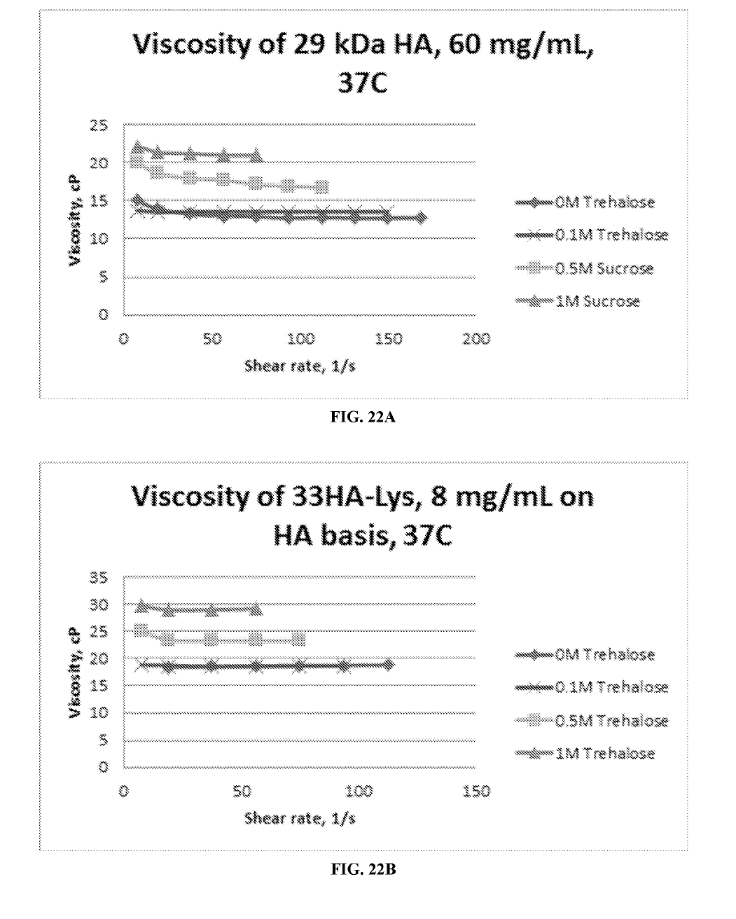

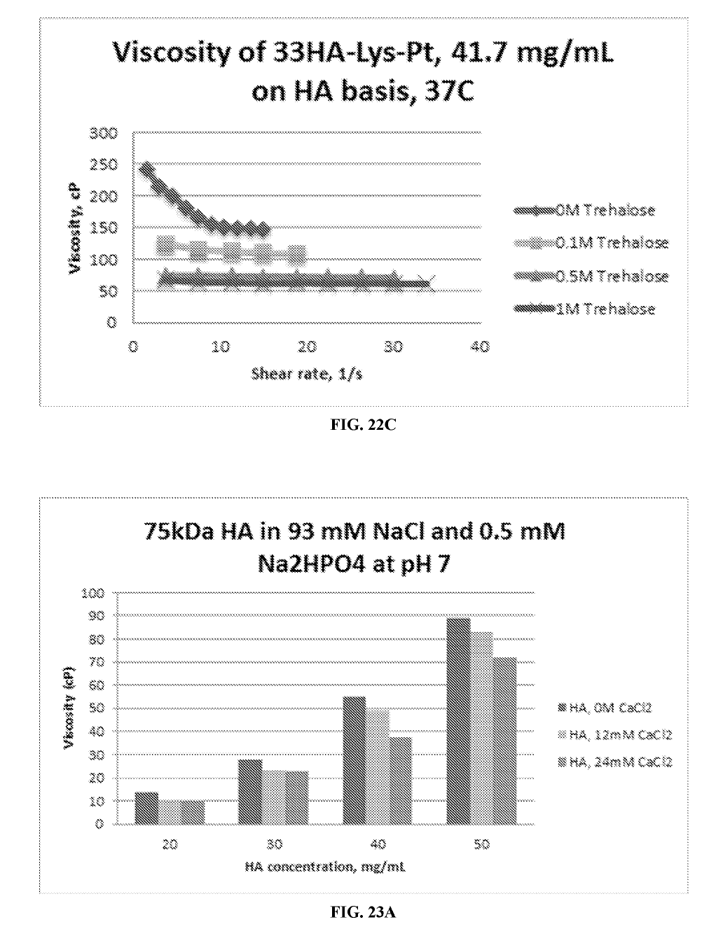

The viscosity of 29 kDa HA (60 mg/mL), 33HA-Lys (33 kDa HA with linker, 8 mg/mL on HA basis), and 33HA-Lys-Pt (41.7 mg/mL on HA basis) with increasing concentration of trehalose (0, 0.1, 0.5 and 1 M) were measured using a rheometer at 37° C. Trehalose (0.1, 0.5 and 1M) increased the viscosity of plain HA and 33HA-Lys, however, it unexpectedly reduced the viscosity of 33HA-Lys-Pt (FIGS. 22A, 22B, and 22C).

### Example 37

### HA-Lys-Pt Viscosity Reduction Using CaCl₂

The effect of calcium chloride in viscosity reduction was evaluated for various batches of HA-Lys-Pt. The viscosity of plain HA was determined in the presence of CaCl₂ and used as a control. The viscosity of HA-Lys-Pt in the presence of different concentrations of CaCl₂ and buffer conditions was measured and compared to plain HA.

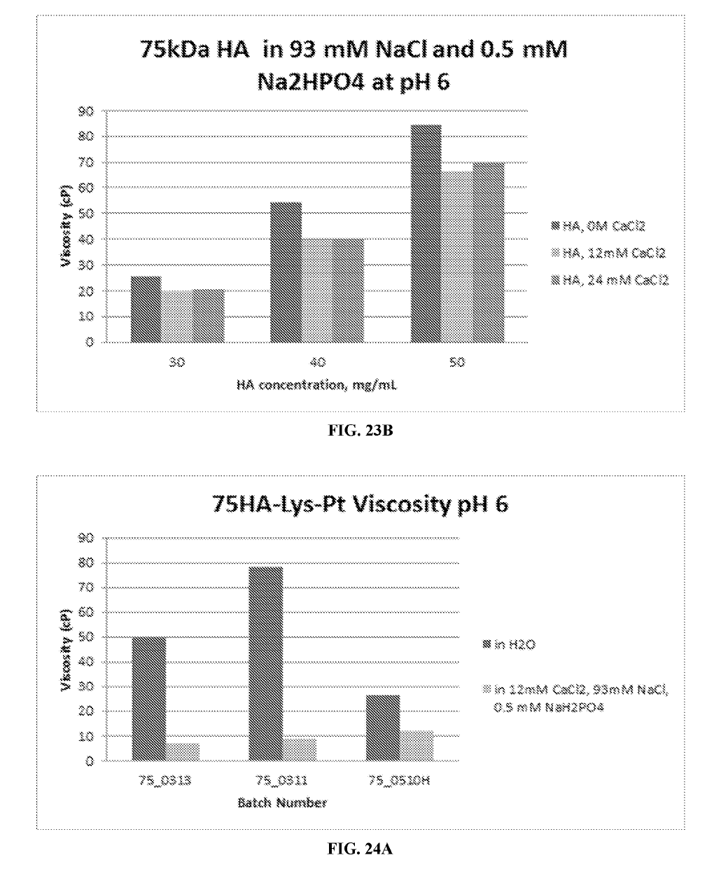

Five hundred microliters of a phosphate buffered saline solution (PBS, 373 mM NaCl, 2 mM Na₂HPO₄, pH 7.1) and 500 μL of CaCl₂ solution (48 and 96 mM, pH 9.0) were added to 1 mL of HA (75 kDa, 60, 90, 120 and 150 mg/mL). The solutions were incubated in the dark for 20 hours using a CLAY ADAMS® Brand Nutator. After 20 hours, the pH of the solutions were adjusted to pH 7.0 using 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 23A. CaCl₂ slightly reduced the viscosity of different concentrations of 75 kDa HA at pH 7.0.

Five hundred microliters of a phosphate buffered saline solution (PBS, 373 mM NaCl, 2 mM Na₂HPO₄, pH 7.1) and 500 μL of CaCl₂ solution (48 and 96 mM, pH 9.0) were added to 1 mL of HA (75 kDa, 90, 120 and 150 mg/mL). The solutions were incubated in the dark for 20 hours using a CLAY ADAMS® Brand Nutator. After 20 hours, the pH of the solutions were adjusted to pH 6.0 using 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 23B. CaCl₂ slightly reduced the viscosity of different concentrations of 75 kDa HA at pH 6.0.

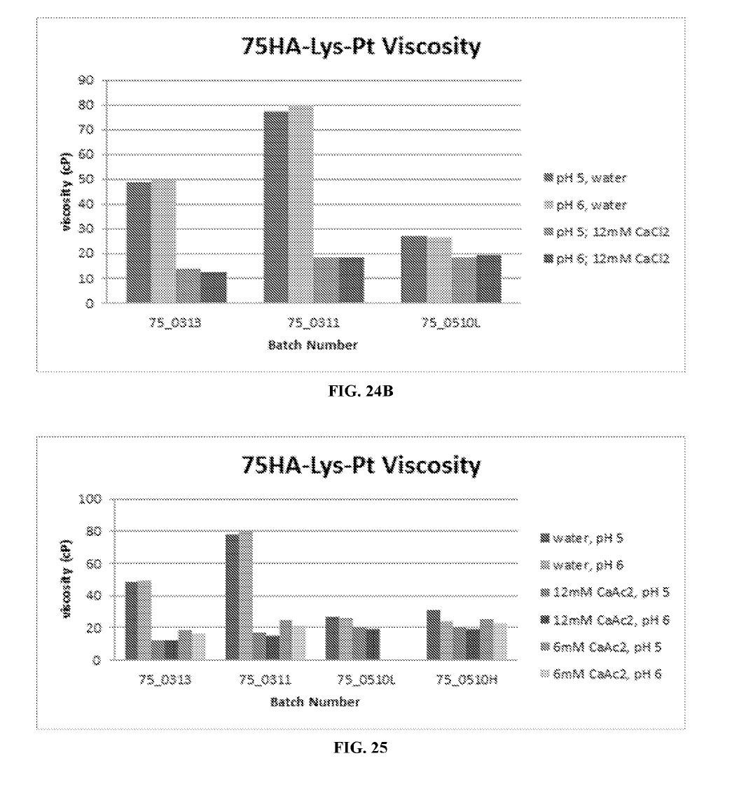

Five hundred microliters of a phosphate buffered saline solution (PBS, 373 mM NaCl, 2 mM Na₂HPO₄, pH 7.1) and 500 μL of CaCl₂ solution (48 mM, pH 9.0) were added to 1 mL of three different batches of 75HA-Lys-Pt. The solutions were incubated in the dark for 20 hours using a CLAY ADAMS® Brand Nutator. After 20 hours, the pH of the solutions were adjusted to pH 6.0 using 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 24A. The CaCl₂-containing buffer greatly reduced the viscosity of all three batches of 75HA-Lys-Pt at pH 6.0. The percent reduction ranged from 55.2-88.6%.

One milliliter of CaCl₂ solution (24 mM, pH 9.0) was added to 1 mL of three different batches of 75HA-Lys-Pt. The solutions were incubated in the dark for 20 hours using a CLAY ADAMS® Brand Nutator. After 20 hours, the pH of the solutions were adjusted to pH 5.0 or 6.0 using 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 24B. The buffer-free CaCl₂ solution greatly reduced the viscosity of all three batches of 75HA-Lys-Pt at pHs 5.0 and 6.0. The percent reduction ranged from 32.1-76.6% at pH 5.0 and 26.6-77.0% at pH 6.0.

### Example 38

### HA-Lys-Pt Viscosity Reduction Using Calcium Acetate

One milliliter of calcium acetate (CaAc₂) solution (12 and 24 mM, pH 9.0) was added to 1 mL of four different batches of 75HA-Lys-Pt. The solutions were incubated in the dark for 20 hours using a CLAY ADAMS® Brand Nutator. After 20 hours, the pH of the solutions were adjusted to pH 5.0 or 6.0 using 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 25. The 12 mM CaAc₂ solution greatly reduced the viscosity of all batches of 75HA-Lys-Pt at pHs 5.0 and 6.0. The percent reduction ranged from 25.1-78.0% at pH 5.0 and 21.4-81.0% at pH 6.0.

### Example 39

### HA-Lys-Pt Viscosity Reduction Using Calcium Lactate

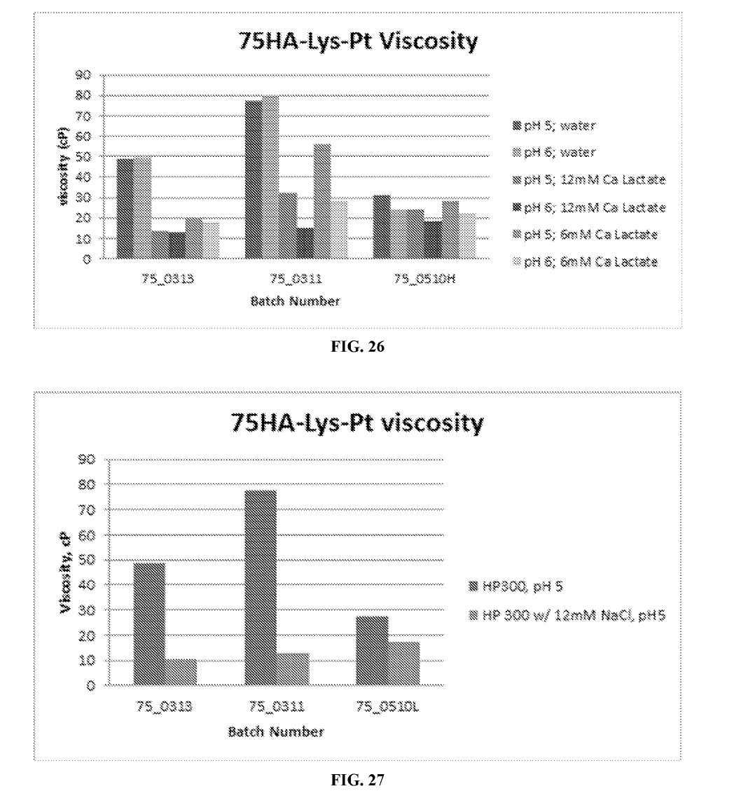

One milliliter of calcium lactate solution (12 and 24 mM, pH 9.0) was added to 1 mL of four different batches of 75HA-Lys-Pt. The solutions were incubated in the dark for 20 hours using a CLAY ADAMS® Brand Nutator. After 20 hours, the pH of the solutions were adjusted to pH 5.0 or 6.0 using 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 26. The 12 mM calcium lactate solution greatly reduced the viscosity of all batches of 75HA-Lys-Pt at pHs 5.0 and 6.0. The percent reduction ranged from 23.0-71.8% at pH 5.0 and 24.3-80.9% at pH 6.0.

### Example 40

### HA-Lys-Pt Viscosity Reduction Using Sodium Chloride

One milliliter of sodium chloride solution (24 mM) was added to 1 mL of three different batches of 75HA-Lys-Pt. The solutions containing 12 mM NaCl were incubated in the dark for 20 hours using a CLAY ADAMS® Brand Nutator. After 20 hours, the pHs of the solutions were adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 27. Sodium chloride greatly reduced the viscosity of all batches of 75HA-Lys-Pt at pH 5.0. The percent reduction ranged from 36.6-83.2% at pH 5.0.

### Example 41

### HA-Lys-Pt Viscosity Reduction Using Sodium Citrate

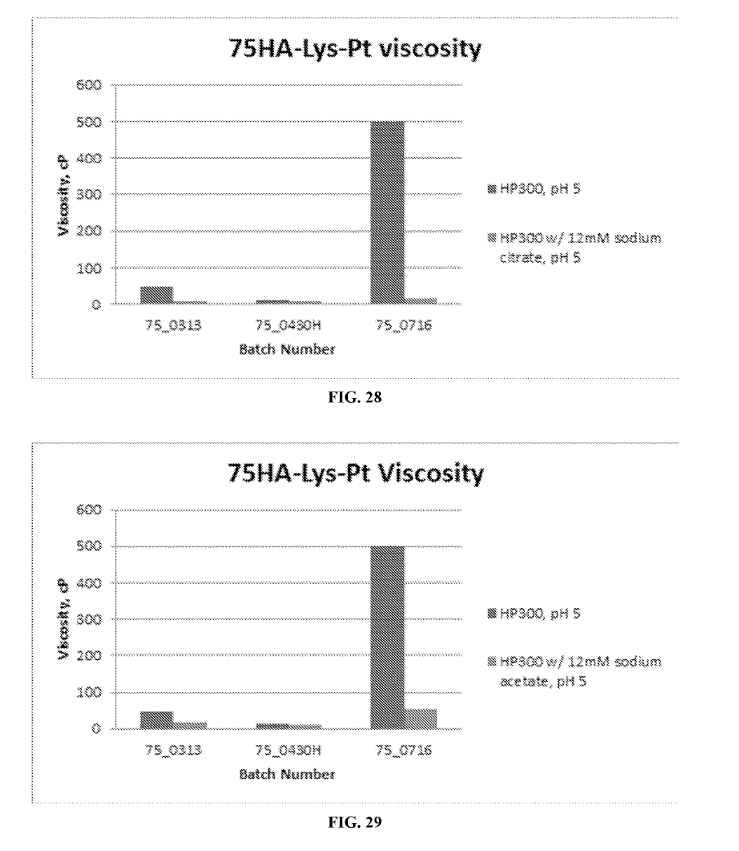

One milliliter of sodium citrate solution (24 mM) was added to 1 mL of three different batches of 75HA-Lys-Pt. The solutions containing 12 mM sodium citrate were incubated in the dark for 20 hours using a CLAY ADAMS® Brand Nutator. After 20 hours, the pH of the solution was adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 28. Sodium citrate greatly reduced the viscosity of all batches of 75HA-Lys-Pt at pH 5.0. The percent reduction ranged from 38.4-97.2% at pH 5.0.

### Example 42

### HA-Lys-Pt Viscosity Reduction Using Sodium Acetate

One milliliter of sodium acetate solution (24 mM) was added to 1 mL of three different batches of 75HA-Lys-Pt. The solutions containing 12 mM sodium acetate were incubated in the dark for 20 hours using a CLAY ADAMS® Brand Nutator. After 20 hours, the pH of the solution was adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 29. Sodium acetate greatly reduced the viscosity of all batches of 75HA-Lys-Pt at pH 5.0. The percent reduction ranged from 14.5-89.2% at pH 5.0.

### Example 43

### HA-Lys-Pt Viscosity Reduction Using Sodium Carbonate

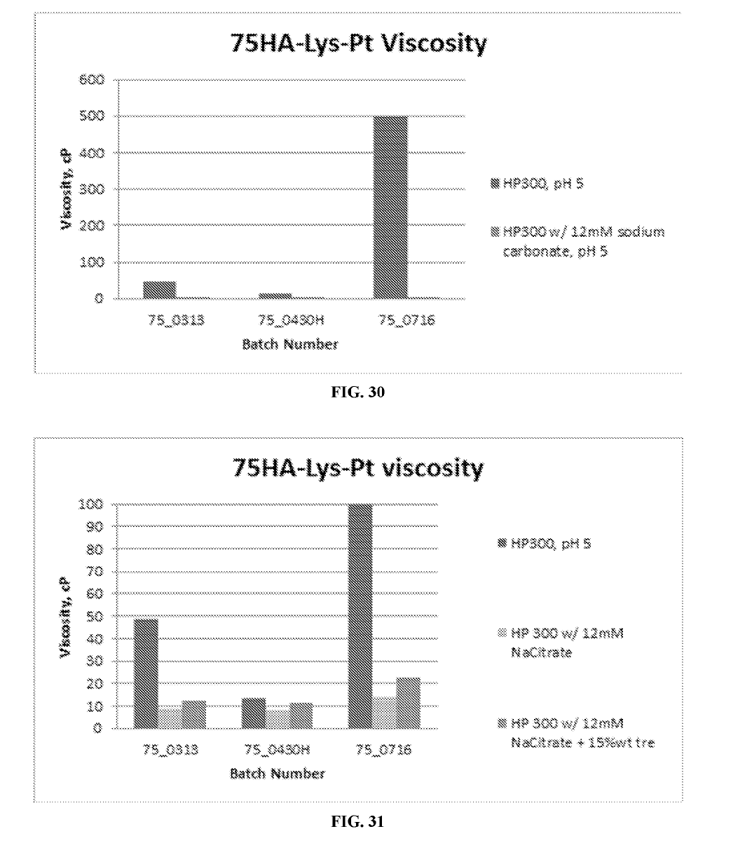

One milliliter of sodium carbonate solution (24 mM) was added to 1 mL of three different batches of 75HA-Lys-Pt. The solutions containing 12 mM sodium carbonate were incubated in the dark for 20 hours using a CLAY ADAMS® Brand Nutator. After 20 hours, the pH of the solution was adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 30. Sodium carbonate greatly reduced the viscosity of all batches of 75HA-Lys-Pt at pH 5.0. The percent reduction ranged from 60.6-99.2% at pH 5.0.

### Example 44

### HA-Lys-Pt Viscosity Reduction Using Sodium Citrate and Trehalose

One milliliter of sodium citrate solution (24 mM) was added to 1 mL of three different batches of 75HA-Lys-Pt. The solutions containing 12 mM sodium citrate were incubated in the dark for 19 hours using a CLAY ADAMS® Brand Nutator. After 19 hours, 300 mg of trehalose (15% wt) was added and the solution was nutated for another 30 min. The pH of the solution was adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 31. Sodium citrate greatly reduced the viscosity of all batches of 75HA-Lys-Pt at pH 5.0. The percent reduction ranged from 15.6-95.4% at pH 5.0.

### Example 45

### HA-Lys-Pt Viscosity Reduction Using Sodium Acetate and Trehalose

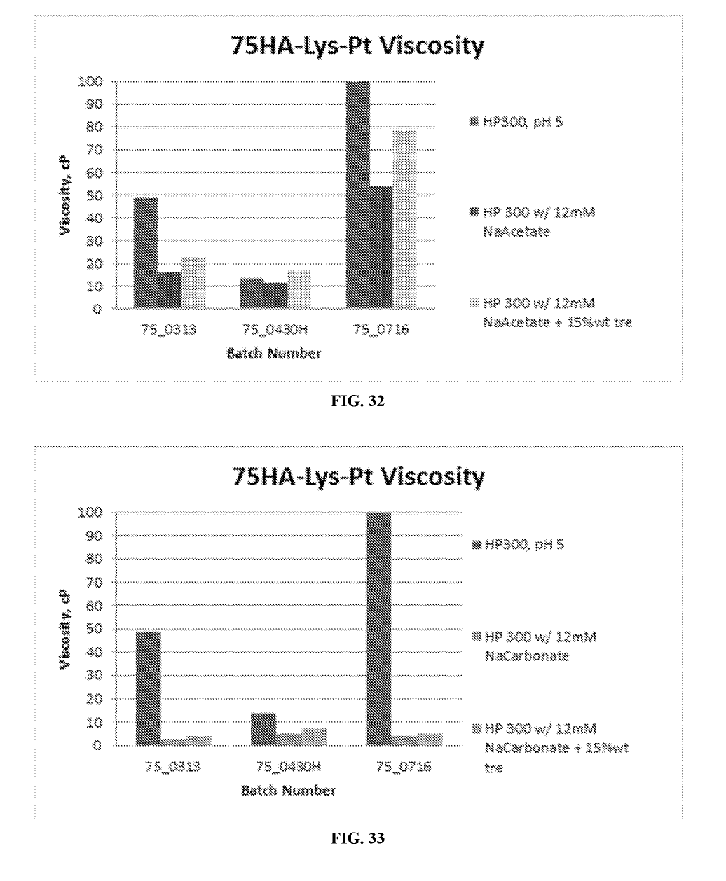

One milliliter of sodium acetate solution (24 mM) was added to 1 mL of two different batches of 75HA-Lys-Pt. The solutions containing 12 mM sodium acetate were incubated in the dark for 20 hours using a CLAY ADAMS® Brand Nutator. After 19 hours, 300 mg of trehalose (15% wt) was added and the solution was nutated for another 30 min. The pH of the solution was adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 32. Sodium acetate and trehalose reduced the viscosity of both batches of 75HA-Lys-Pt at pH 5.0. The percent reduction was 54.1% and 84.3% at pH 5.0.

### Example 46

### HA-Lys-Pt Viscosity Reduction Using Sodium Carbonate and Trehalose

One milliliter of sodium carbonate solution (24 mM) was added to 1 mL of three different batches of 75HA-Lys-Pt. The solutions containing 12 mM sodium carbonate were incubated in the dark for 20 hours using a CLAY ADAMS® Brand Nutator. After 19 hours, 300 mg of trehalose (15% wt) was added and the solution was nutated for another 30 min. The pH of the solution was adjusted to pH 5.0 1N NaOH. A Brookfield DVIII Ultra Programmable Rheometer was used to measure the viscosity of three 500-μL samples of each solution. Measurements were taken for each at three different RPMs, where the torque ranged from 10-100%. Values for viscosity were not recorded until at least 30 seconds after the motor was started, after the torque readings were stabilized. The result is shown in FIG. 33. Sodium carbonate and trehalose greatly reduced the viscosity of all batches of 75HA-Lys-Pt at pH 5.0. The percent reduction ranged from 48.5-99.0% at pH 5.0.

### Example 47

### HA-Lys-Pt Viscosity Reduction Using Sodium Chelex 100

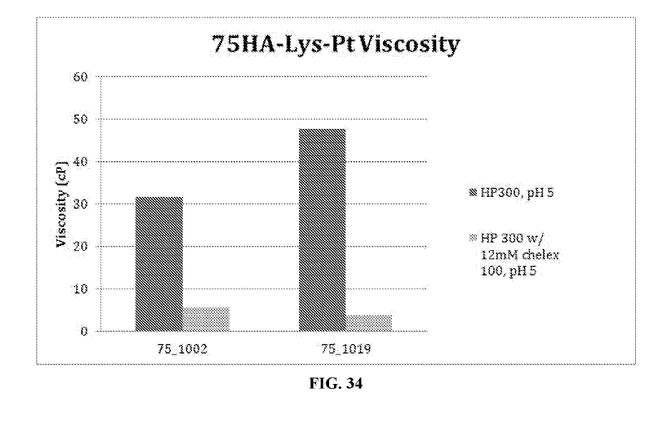

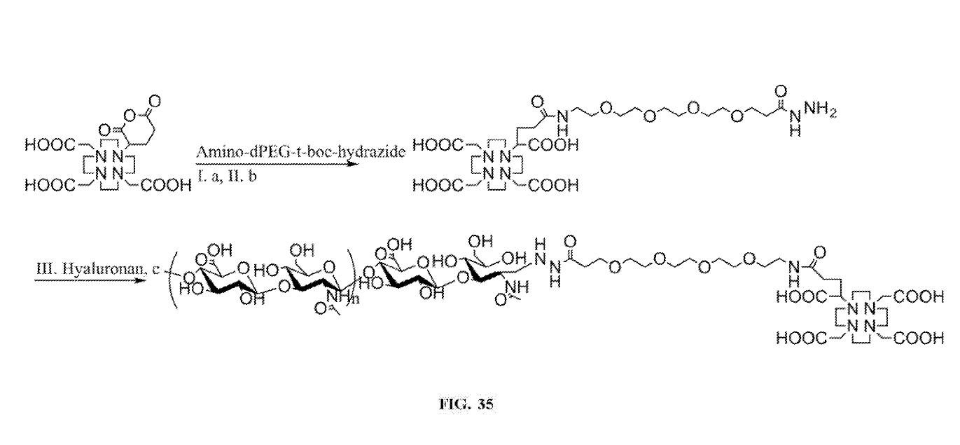

Forty milligrams of Sodium Chelex® 100 (0.024 meq) was added to 1 mL of two different batches of 75HA-Lys-Pt. The solutions were incubated in the dark for 20 hours using a CLAY ADAMS® Brand Nutator. The pH of the solution was measured to be pH 6.0, and the solution was diluted two-fold using water and pH was adjusted to pH 5.0 using 1-N NaOH. A Brookfield DVIII Ultra Programmable Rheometer was used to measure the viscosity of 500-μL samples of each solution. Measurements were taken for each at three different RPMs, where the torque ranged from 10-100%. Values for viscosity were not recorded until at least 30 seconds after the motor was started, after the torque readings were stabilized. Sodium Chelex® 100 reduced the viscosity of both batches of 75HA-Lys-Pt at pH 5.0. The percent reduction was 82.0% and 91.7% at pH 5.0. The result is shown in FIG. 34.

### Example 48

### Toxicity of HA-Lys-Pt w/24 mM Sodium Chelex® 100 in Mice

The toxicity of HA-Lys-Pt with 24 mM Sodium Chelex 100 was evaluated in blab/C mice. Two blab/C mice were injected subcutaneously with 40 mg/kg HP300w/24 mM Sodium Chelex® 100. The body weights of the animals were checked daily. Two mice were found dead 4 and 7 days post-injection. Acute toxic reactions including lethargy, paralysis, and respiratory depression were not observed.

### Example 49

### Preparation and Characterization of HA-PEG-DOTAGA Conjugate

A solution of DOTAGA anhydride (100 mg, 0.218 mmol) in 4-mL of anhydrous DMF was mixed with a solution of amino-PEG-t-boc-hydrazide (165.5 mg, 0.436 mmol, 2 eq.) in 1-mL of anhydrous DMF in the presence of triethylamine (121.6 μL, 4 eq.). The reaction was allowed to proceed at 50° C. under argon for 24 hours. The solvent was removed under reduced pressure to give the desired product a yellowish colored oil, which was used without further purification. HRMS (ESI): m/z: calculated for C₃₅H₆₃N₇O₁₆+H⁺/Na⁺: 838.4410/860.4229; found: 838.4356/860.4232. The de-protection of t-boc was completed in 2-mL of TFA at 0° C. in an ice bath for 3 hours. After the removal of TFA, the crude solid was purified by a C18 reverse-phase column (Discovery HS F5 and conventional C18, 15 cm×4.6 mm). Mobile phase A: ACN; B: H₂O+0.4% HCOOH (0-10 min, B: 90-60%); flow rate: 0.8 mL/min; column temperature: 40° C. The desired compound was detected using a UV detector at 250 nm. The fraction at 6.7 min was collected and dried. HRMS (ESI): m/z: calcd for C₃₀H₅₅N₇O₁₄+H⁺: 738.3815; found: 738.3856.

Sodium hyaluronate (35 or 75 kDa, 100 mg) was dissolved in 5-mL of acetate buffer (10 mM, pH 5.6) to a homogeneous solution. After DOTAGA-PEG (45 mg, 21 eq.) and sodium cyanoborohydride (NaBH₃CN, 13.5 mg, 75 eq.) were added, the mixture was stirred at 50° C. for two day under argon (FIG. 35). The resulting solution was filtered through a nylon syringe filter (0.22-μm) and purified by dialysis (MWCO10,000) against ddH₂O with four water changes in 2 days. The degree of substitution was determined to be 1.2% on molar basis by ¹H-NMR (400 MHz, D₂O) δ=4.46 (d, J=7.6 Hz, 1H), 4.38 (d, J=6.5 Hz, 1H), 3.74-3.25 (m, 10H), 2.55 (m, 2H), 1.93 (s, 3H).

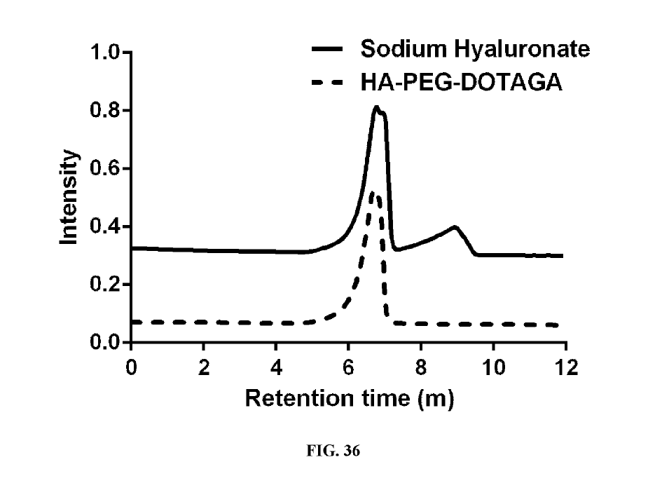

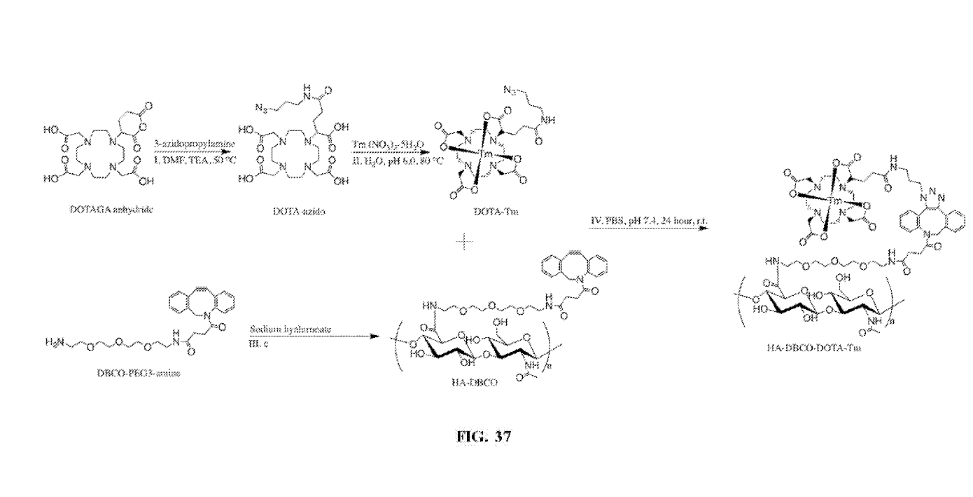

Due to previous reports that HA depolymerisation could be produced chemically by reducing agents, such as ascorbic acid with or without catalysts, the molecular weight distribution of the HA-PEG-DOTAGA nanoconjugate was characterized by GFC, and was compared to that of sodium hyaluronate, on a Shimadzu 2010CHT HPLC with a refractive index (RI) detector (Shimadzu RID-10A) and UV detector at 210 nm. GFC was performed with a Shodex OHpak SB-804 HQ column (Showa Denko America, Inc., New York, N.Y.) at 40° C. using ddH₂O as the mobile phase at a flow rate of 0.8 mL/min. The HA-PEG-DOTAGA conjugate exhibited the same retention time on the GFC column as that of sodium hyaluronate (FIG. 36), demonstrating that the use of NaBH₃CN did not induce any damage to HA backbone.

### Example 50

### Cu Labeling of HA-PEG-DOTA-GA

HA-PEG-DOTA-GA (35 or 75 kDa, 10 mg) was dissolved in 800 μL of sodium acetate buffer (0.1 M, pH 5.5). No less than 14 mCi or 518 MBq of ⁶⁴Cu in 0.1-N HCl solution was added. The reaction was allowed to proceed at 47° C. for 1 hour with stirring. After the reaction mixture cooled down to room temperature, it was first purified using a centrifugal filter unit (MWCO 10,000). Briefly, the reaction mixture was diluted with 1 mL of PBS and transferred to the filter unit, which was then put in a centrifuge and spun at 4,000 rpm for 10 min. The product solution was washed three times with 1-mL PBS each time by repeating centrifugal filtrations. HA-PEG-DOTA-⁶⁴Cu was further purified by passing through an equilibrated PD-10 desalting column using PBS as eluent. The PD-10 column was equilibrated with 25-mL PBS 24 hours before use. One milliliter of HA-PEG-DOTA-⁶⁴Cu solution post centrifugation was transferred to the column. After the solution flowed down the gel surface, another 10 mL of PBS was added to elute the HA-PEG-DOTA-⁶⁴Cu. The eluate was collected in 2-mL tubes with 500-μL aliquot in each tube.

A mock experiment was conducted using 75-kDa HA-PEG-DOTA-GA (25 mg) and CuCl₂ (0.25 mg). Copper concentration and polymer content in collected fractions from the PD-10 column were measured using ICP-MS and GFC (Table 9), respectively. Due to low copper concentration in fractions #6-7 and a significant decrease in polymer content in fraction #14, fractions #8-13 were combined to obtain HA-PEG-DOTA-Cu solution in PBS.

Table 9 shows the copper concentration and polymer content in fractions.

TABLE 9

| | Copper concentration, | |
|---|---|---|
| Fractions | μg/mL | Peak area in SEC |
| #6 | 0.066 | 44918 |
| #7 | 2.589 | 349227 |
| #8 | 8.91 | 7541475 |
| #9 | 11.94 | 7059930 |
| #10 | 9.462 | 6203673 |
| #11 | 10.242 | 5858601 |
| #12 | 11.247 | 5378168 |
| #13 | 11.901 | 4149445 |
| #14 | 9.777 | 450421 |

### Example 51

### Lyophilization of 33HA-Lys-Pt

33HA-Lys-Pt (MW of HA: 33 kDa) from batch 33_01015 was used to evaluate the impact of trehalose as an excipient and lyoprotectant. Increasing percent weights of trehalose (0, 5, 10, and 15% wt) was added into four 500-μL solution of 33HA-Lys-Pt (3.1 mg/mL on cisplatin basis), subsequently pH was adjusted to 6 or 7. The solution was freeze-dried using the following program. Samples were frozen in a −80° C. freezer for 4 hours. The vials were transferred to a freeze dryer and kept at −40° C. for 1.5 hours. The condenser point was set at −70° C. The vacuum setpoint was 100 mTorr. The parameters for primary drying were 30 hours at −35° C. and 10 mTorr. The parameters for secondary drying were 5 hours at 2° C. and 10 mTorr, followed by another 5 hours at 8° C. and 10 mTorr. Once the drying was complete, vials containing lyophilized 33HA-Lys-Pt samples were stored at −20° C. in the dark until rehydration test within 24 hours. During rehydration, 500-μL of water was added to the vial and the sample was gently shaken. The rehydration time was reported in Table 10. The small molecular weight platinum species such as mono-aquated and di-aquated platinum were separated from the reconstituted 33HA-Lys-Pt using a Centricon centrifugal filter unit (10,000 MWCO, 4000 rpm for 10 min at 4° C.) and analyzed by an HPLC assay. The mobile phase was sodium phosphate solution (10-mM, pH 3.0) at a flow rate of 0.25 mL/min on an Alltech® 250 mm×4.6 mm M/M RP8/cation column thermostated at 25° C. The internal standard, a mixture of platinum species containing cisplatin, monoaquated and diaquated platinum, was prepared by dissolving 10 mg of cisplatin in 10 mL of water and stirring for 48 hours. A series of cisplatin solutions in saline were used as calibration standards. The peaks of cisplatin, Pt-monoaqua, and Pt-diaqua were detected by a UV detector at 283 nm. Diaqua platinum was detected under this condition.

Table 10 reported the rehydration time of eight 33HA-Lys-Pt samples (3.1 mg/mL on cisplatin basis) with different pHs and trehalose contents.

TABLE 10

| | Trehalose | Rehydration Time, |
|---|---|---|
| pH | % wt | hour |
| 6 | 0 | Ca. 24 |
| | 5 | Ca. 12 |
| | 10 | 6 |
| | 15 | 6 |
| 7 | 0 | 7 |
| | 5 | 1 |
| | 10 | 0.5 |
| | 15 | 0.5 |

### Example 52

### Lyophilization of 75HA-Lys-Pt

75HA-Lys-Pt (MW of HA: 75 kDa) from batch 75_1205 was used to evaluate the impact of trehalose as an excipient and lyoprotectant. Increasing percent weights of trehalose (0, 5, 10, and 15% wt) was added into 500-μL solution of 75HA-Lys-Pt (1.46 mg/mL on cisplatin basis), subsequently pH was adjusted to 6 or 7. The solution was freeze-dried using the following program. Samples were frozen in a −80° C. freezer for 4 hours. The vials were transferred to a freeze dryer and kept at −40° C. for 1.5 hours. The condenser point was set at −70° C. The vacuum setpoint was 100 mTorr. The parameters for primary drying were 30 hours at −35° C. and 10 mTorr. The parameters for secondary drying were 5 hours at 2° C. and 10 mTorr, followed by another 5 hours at 8° C. and 10 mTorr. Once the drying was complete, vials containing lyophilized 75HA-Lys-Pt samples were stored at −20° C. in the dark until rehydration test within 24 hours. During rehydration, 500-μL of water was added to the vial and the sample was gently shaken. The rehydration time was reported in Table 11.

75HA-Lys-Pt (MW of HA: 75 kDa) with increasing cisplatin contents (from 0.86 to 3.00 mg/mL) from batches 75_0331 (cisplatin conc=0.86 mg/mL), 75_0419 (cisplatin conc=1.44 mg/mL), 75_0430 (cisplatin conc=1.99 mg/mL), 75_0510 (cisplatin conc=2.32 mg/mL), and 75_0510H (cisplatin conc=3.00 mg/mL) were evaluated for rehydration time and diaqua content in the presence of different amounts of trehalose and at different pHs. One milliliter of samples were used for all tests. The trehalose percent weights varied from 8-20% wt. The pHs included 4.5, 5, 6, and 7. The solution was freeze-dried using the aforementioned freeze-drying program. Once the drying was complete, vials containing lyophilized 75HA-Lys-Pt samples were stored at −20° C. in the dark until rehydration test within 24 hours. During rehydration, 1-mL of water was added to the vial and the sample was gently shaken. Diaquated platinum species were separated using the aforementioned Centricon units and analyzed using the aforementioned HPLC method. The rehydration time and the detection of diaqua were reported in Table 12.

Other sample freezing methods were also tested. For examples, samples were gradually frozen in ethanol bath at −80° C. and lyophilized. In addition, samples were flash frozen using liquid nitrogen and lyophilized. Results were summarized in Table 13.

Rehydration using one half of the original sample volume was tested. For example, a 75HA-Lys-Pt sample (2 mL) was freeze-dried and rehydrated using 1 mL of water. Result was reported in Table 13.

Besides trehalose, PEG 200 (2 and 5% wt) was tested as a second excipient to aid the rehydrate process. Result was reported in Table 13.

Table 11 reported the rehydration time of 75HA-Lys-Pt samples (1.46 mg/mL) with different pHs and trehalose contents.

TABLE 11

| | Trehalose | Rehydration Time, |
|---|---|---|
| pH | % wt | hour |
| 6 | 0 | >48 |
| | 5 | Ca. 12 |
| | 10 | 7 |
| | 15 | 6 |
| 7 | 5 | ca. 2.5 |
| | 10 | 1 |
| | 15 | 1 |

Table 12 reported the rehydration time and diaqua detection of 75HA-Lys-Pt samples (0.86-3.00 mg/mL) with different pHs and trehalose contents.

TABLE 12

| | Cisplatin | | Treha- | Rehy- | Presence of |
|---|---|---|---|---|---|
| Batch | Conc, | | lose | dration | diaqua? |
| No | mg/mL | pH | % wt | Time, h | (Y/N) |
| 75_0331 | 0.86 | 4.5 | 13 | 5 | N |
| | | 6 | 13 | 2.5 | Y |
| | | 7 | 13 | 2 | Y |
| 75_0419 | 1.44 | 4.5 | 8 | 8-20 | N |
| | | 6 | | 8-20 | N |
| | | 7 | | 8-20 | Y |
| | | 4.5 | 10 | 8-20 | N |
| | | 6 | | 8-20 | N |
| | | 7 | | 8-20 | Y |
| 75_0430 | 1.99 | 4.5 | 15 | 26 | N |
| | | 4.5 | 20 | 26 | N |
| | | 5 | 15 | 26 | N |
| | | 5 | 20 | 26 | N |
| | | Adjusted | 10 | 6-20 | N |
| | | to 5 at | | | |
| | | rehydration | | | |
| | | Adjusted | 10 | 6-20 | N |
| | | to 6 at | | | |
| | | rehydration | | | |
| | | Adjusted | 10 | 6-20 | Y |
| | | to 7 at | | | |
| | | rehydration | | | |
| 75_0510 | 2.32 | 5 | 15 | 24 | N |
| | | 5 | 20 | 24 | N |
| 75_0510H | 3 | 5 | 15 | >48 | Not |
| | | | | | tested |

Table 13 reported the rehydration time of 75HA-Lys-Pt samples (batch 75_0706) with different trehalose contents.

TABLE 13

| Cisplatin | | Trehalose, | Rehydration | |
|---|---|---|---|---|
| Conc, mg/mL | pH | % wt | Time, hours | Comment |
| 3.00 | 5 | 15 | >48 | N/A |
| 2.13 | 5 | 15 | >48 | Ethanol bath |
| | | 20 | >48 | Ethanol bath |
| | | 15 | >24 | Liquid nitrogen |
| | | 20 | >24 | Liquid nitrogen |
| 1.06 | 5 | 7.5 | 7 | Rehydration |
| | | 10 | 7 | using half the |
| | | | | original volume |
| 2.13 | 5 | 20 (PEG = 2% wt) | 48 | PEG200 |
| | | 20 (PEG = 5% wt) | 48 | PEG200 |

### Example 53

### Preparation of HA-DBCO-DOTA-Tm Conjugate

Fifty milligrams of sodium hyaluronate (75 kDa, 0.12 mmol) was dissolved in 2.5 mL of water, followed by addition of DibenzocyclooctyneBCO-PEG3-amine (3.2 mg, 6.6 Conju-Probe LLC., San Diego, Calif.) and DMTMM (2.5 mg, 9.0 μmol). The pH was adjusted to 4.5 using 1-N HCl, the mixture was stirred at 30° C. for 48 hours in the dark to form HA-DBCO with a DBCO substitution degree of 0.5 mol %. The resulting solution was neutralized using 1-N NaOH, filtered using a syringe filter (PES, 0.22-μm), dialyzed against water (Thermo Scientific™, SnakeSkin™ Dialysis Tubing, MWCO 10,000 Da) for 48 hours and freeze-dried (Labconco 2.5 Plus FreeZone, Kansas City, Mo.). ¹H-NMR (400 MHz, D₂O) δ (ppm): 7.44-7.28 (m, 8H), 5.10 (d, 1H), 4.46 (d, J=6.8 Hz, 1H), 4.36 (d, J=6.8 Hz, 1H), 3.74-3.25 (m, 10H), 2.54-2.44 (m, 2H), 1.92 (s, 3H).

A solution of DOTAGA anhydride (300 mg, 0.65 mmol) in 10 mL of anhydrous DMF was mixed with a solution of 3-azidopropylamine (131 mg, 1.3 mmol) in 1-mL anhydrous DMF in the presence of triethylamine (TEA, 365 μL, 2.6 mg). The reaction was allowed to proceed at 50° C. under argon for 24 hours. The solvent was removed under reduced pressure to obtain a yellowish oil of crude DOTA-azido, which was then purified by recrystallization in a mixture of methanol and diethyl ether. HRMS (ESI): m/z: calculated for C₃₅H₆₃N₇O₁₆+H⁺/Na⁺: 559.2840/581.2659; found: 559.2803/581.2632.

To synthesize DOTA-Tm complex, 100 mg of DOTA-azido (0.18 mmol) was dissolved in 2-mL water, and the solution was adjusted to pH 7.0 using 1-N NaOH. The Tm(III) (Tm(NO₃)₃.5H₂O, 178 mg, 0.40 mmol) solution in 1.5 mL water was added dropwise in 3 hours, after the pH of the solution was checked and adjusted to 6.0. The reaction mixture was then stirred at 80° C. for 4 hours and room temperature for 24 hours in the dark. The excess Tm(III) was removed by raising the pH to 9.0 to precipitate excess metal as hydroxide. The suspension was spun at 4,000 rpm for 20 min at 4° C., and the supernatant was collected and freeze-dried. The resulting yellowish solid was then recrystallized in a mixture of methanol and diethyl ether, and solvent residual was removed under reduced pressure. The product was finally re-dissolved in water and subsequently freeze-dried to yield DOTA-Tm as an off-white solid. FIRMS (ESI): m/z: calculated for C₂₂H₃₄N₈O₁₉Tm⁻: 723.1791; found: 723.1754. The synthesis scheme is depicted in FIG. 37.

The HA-DBCO-DOTA-Tm conjugate was prepared by incubating HA-DBCO (20 mg, 0.26-μmol DBCO) and DOTA-Tm (580 μg, 0.78 μmol) in PBS (Phosphate Buffer Saline) at room temperature for 24 hours. In the meanwhile, sodium hyaluronate (28 mg) and Tm(NO₃)₃.5H₂O (1.5 mg) were mixed in 1 mL of PBS at room temperature for 24 hours to prepare a HA-Tm conjugate. The products were purified by dialysis against sodium phosphate buffer (10 mM, pH 7.4) for 24 hours using 10 kDa MWCO dialysis tubing at room temperature. The loading degrees (LD) of Tm(III) on HA-DBCO-DOTA-Tm and HA-Tm was determined by the ICP-MS to be 0.04-0.06 wt % and 0.0004 wt %, respectively, on Tm(III) weight basis.

### Example 54

### Characterization of HA-DBCO-DOTA-Tm Conjugate

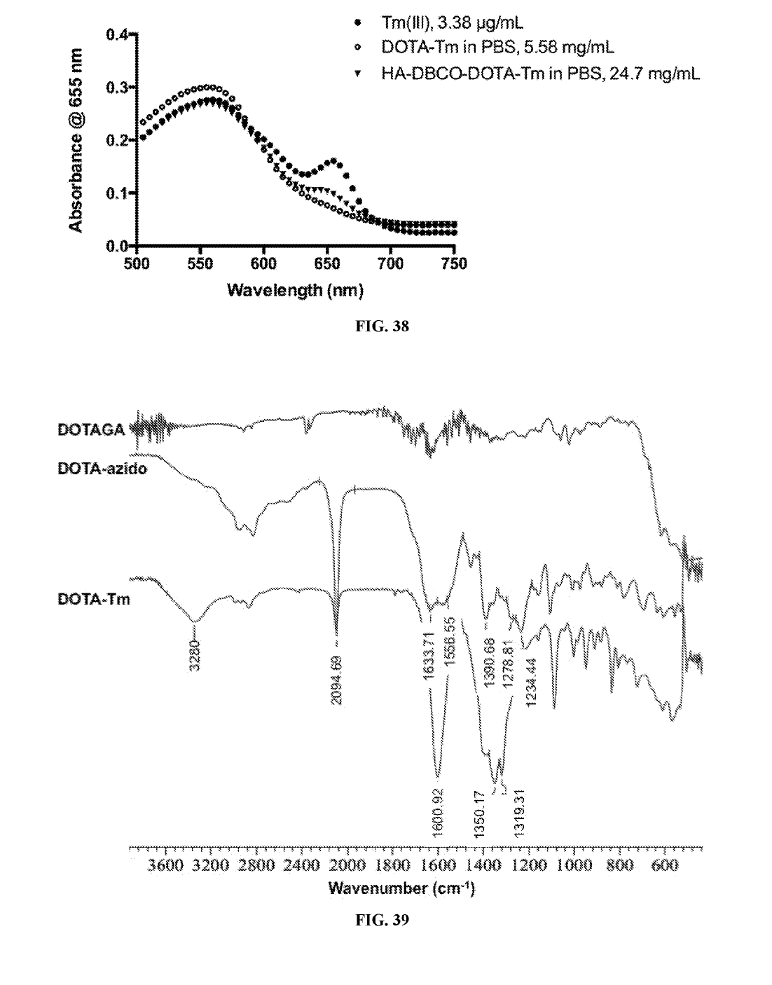

The intermediate, DOTA-Tm, and the product, HA-DBCO-DOTA-Tm, were evaluated for the presence of free Tm(III) ions with an Arsenazo III color test. A solution of Arsenazo III was prepared in 2-mM Tris/1-mM acetate buffer at a concentration of 25 nM. Standard solutions (0-8.45 μg/mL on Tm(III) basis) were prepared by dissolving Tm(NO₃)₃.5H₂O in PBS. For the assay, 25 μL of samples or standard solutions was added into each well of a 96-well plate. To each well, 100 μL of Arsenazo III solution (25 nM) was then added, followed by 5-min shaking at 100 rpm. Absorbance at 655 nm was measured to quantify the concentration of free Tm(III) ions. As shown in FIG. 38, no free Tm(III) was observed in DOTA-Tm solution with a Tm(III) concentration of 5.58 mg/mL. Moreover, in a solution of HA-DBCO-DOTA-Tm in PBS, small amount of free Tm(III) was detected at 0.18 μg/mL. However, the free Tm(III) was less than 0.8% of total Tm(III) in the HA-DBCO-DOTA-Tm solution (24.7 μg/mL).

Characteristic peaks in Fourier-transform infrared spectra (IRAffinity-1 FTIR Spectrophotometer, Shimadzu) confirmed the successful synthesis of DOTA-azido and DOTA-Tm. In FIG. 39, both DOTA-azido and DOTA-Tm showed expected characteristic peak for the azide functional groups at 2094 cm⁻¹. The peaks at 1633 and 1390 cm⁻¹ are assigned to the anti-symmetric and symmetric stretching modes, respectively, of ionized COOH groups of the DOTA-azido. Three bands at 1556, 1633 and 1390 cm⁻¹ are significantly lowered in the DOTA-Tm, in addition to the appearance of strong band at 1601 cm⁻¹, are ascribed to the complexation of Tm(III) ions with COO⁻ groups of the DOTA⁴⁻ ligand. The broad band centered at 3280 cm⁻¹ could be assigned to the coordinated water molecules, implying the presence of the [Tm(DOTA)H₂O] complex.

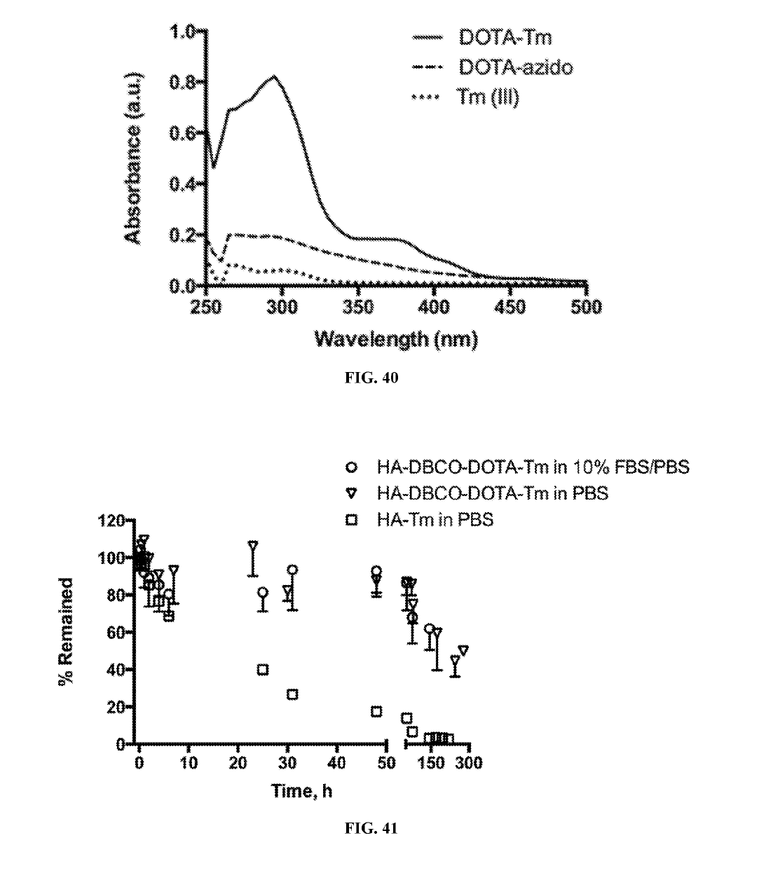

UV/Vis spectrophotometry (Molecular Devices SpectraMax Plus, Sunnyvale, Calif.) was used to verify the formation of DOTA-Tm complex. Sample solutions were prepare by dissolving DOTA-azido, DOTA-Tm and Tm(NO₃)₃.5H₂O [Tm(III)] in PBS, and placed in a 10 mm×10 mm quartz cuvette. The absorption spectra of DOTA-azido, DOTA-Tm and Tm(III) are shown in FIG. 40. Compared with DOTA-azido and Tm(III), DOTA-Tm complex exhibits characteristic peaks at 295 and 385 nm, which indicates that the “in cage” complex formed.

The in vitro release rates of Tm(III) from the HA-DBCO-DOTA-Tm and HA-Tm conjugates were studied using a dialysis method. Typically, 3 mL of conjugate solution was added into the dialysis tubing (MWCO 10,000 Da) and then placed in a 4.0-L PBS (pH 7.4) or a 2-L 10% FBS in PBS solution at 37° C. with stirring at a speed of 250 rpm. The bath volume was replaced every 24 hours to maintain the sink condition. A 100-μL aliquot was withdrawn from the dialysis tubing at the predetermined timepoints. The Tm(III) concentrations in each sample were determined by the ICP-MS analysis. As shown in FIG. 41, Tm(III) was released sustainably from the HA-DBCO-DOTA-Tm conjugate in both PBS and 10% FBS, and its releasing pattern could be fit into a firs-order release kinetics with release half-lives of approximately 6.8 and 2.5 days in PBS and 10% FBS, respectively. In comparison, HA-Tm complex exhibited a relatively faster release of Tm(III) in PBS with a release half-life of approximately 15 hours.

Sprague Dawley rats (300-350 g body weight) were randomly divided to two groups, including a group treated with HA-DBCO-DOTA-Tm (17.84 μg/mL, N=1 per timepoint) and a group treated with DOTA-Tm (107.1 μg/mL, N=1 per timepoint). At each timepoint, drugs were administered subcutaneously into lower cheeks with a single dose of 0.03 mg/kg body weight on Tm(III) basis under anesthesia with 2% isoflurane in oxygen. The animals were euthanized at 2, 6, 24, 48 and 72 hours post injection. Whole blood was drawn and centrifuged at 2,000 g for 5 minutes to collect the plasma. To determine the Tm(III) level in plasma, the plasma samples were diluted using 1%-HNO₃ and analyzed by the ICP-MS. In the plasma samples, peak concentration of Tm(III) in HA-DBCO-DOTA-Tm treated group following the subcutaneous administration occurred at 6 hours, and the concentration in the plasma decreased gradually to near-baseline level within 72 hours post-injection (FIG. 42). In comparison, the concentrations of Tm(III) in the plasma of rats treated with DOTA-Tm decreased in a much faster rate, in which Tm(III) was completely cleared from the blood circulation within 4 hours post-injection.

### Example 55

### Large Scale HA-Lys-Pt Synthesis: Synthesis of 5-Bromoacetyl-2-hydroxybenzoic Acid Methyl Ester

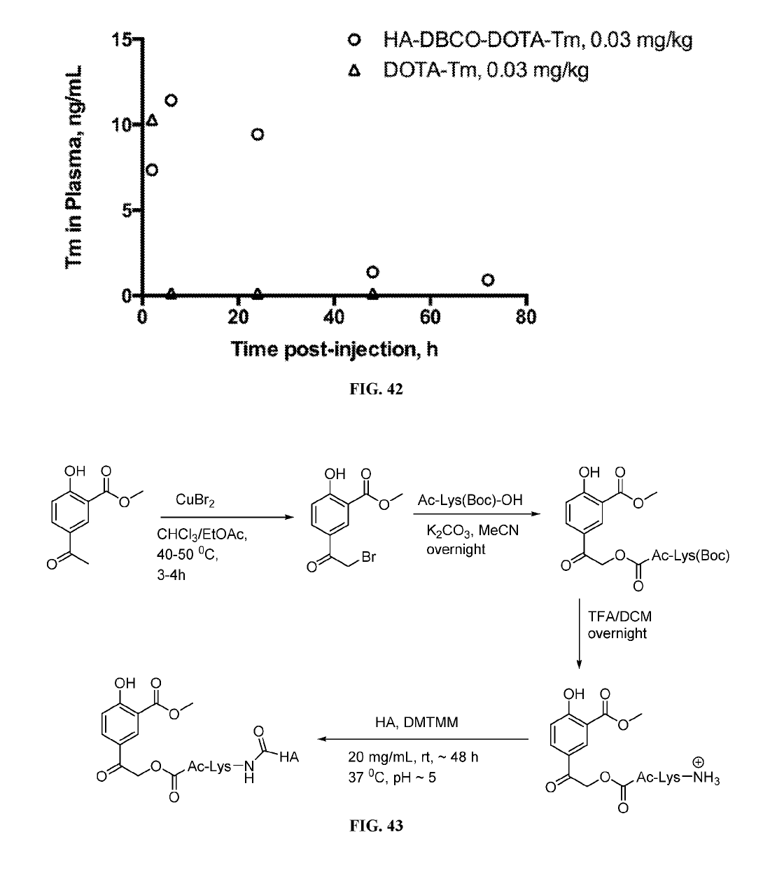

To a stirred solution of methyl 5-acetylsalicylate (1.0 g, 5.1 mmol) in CHCl₃/EtOAc (40 mL) was added copper (II) bromide (2.4 g, 10.8 mmol). The reaction mixture was gently refluxed for 4 hours at 40-50° C. The progress of the reaction was monitored by TLC. Upon completion of the reaction, the mixture was filtered, water (50 mL) and EtOAc (40 mL) were added to the reaction mixture. The EtOAc layer was separated and aqueous layer was re-extracted with EtOAc (20 mL×2). The EtOAc fractions were combined, dried over anhydrous MgSO₄, filtered, and the supernatant concentrated under reduced pressure to afford the crude 5-bromoacetyl-2-hydroxybenzoic acid methyl ester as a yellowish white solid. Crude product was recrystallized using methylene chloride and hexane to afford the title compound as a white solid (1.2 g, 85%). Spectroscopic data are in strong agreement with reported data. ¹H NMR (400 MHz, CDCl3) δ (ppm): 11.35 (s, 1H), 8.53 (d, J=2.3 Hz, 1H), 8.12 (dd, J=8.8, 2.2 Hz, 1H), 7.08 (d, J=8.8 Hz, 1H), 4.41 (s, 2H), 4.02 (s, 3H). FIG. 43 shows the reaction scheme for Examples 55-57.

### Example 56

### Large Scale HA-Lys-Pt Synthesis: Synthesis of Methyl 5-(2-((acetyllysyl)oxy)acetyl)-2-hydroxybenzoate (pHP-Ac-Lys)

To a stirred solution of Ac-Lys(Boc)-OH (1.0 g, 3.5 mmol) in MeCN (40 mL) was added K₂CO₃ (1.4 g, 10.5 mmol). The reaction mixture was stirred for 10 minutes at room temperature. A solution of 5-bromoacetyl-2-hydroxybenzoic acid methyl ester (0.96 g, 3.5 mmol) in MeCN (10 mL) was added dropwise to the reaction mixture. The reaction mixture was stirred under ambient conditions. The progress of the reaction was monitored by TLC. After completion of the reaction, the reaction mixture was filtered and water (50 mL) and EtOAc (40 mL) were added to the reaction mixture. EtOAc layer was separated and aqueous layer was re-extracted with EtOAc (20 mL×2). The EtOAc fractions were combined, dried over anhydrous MgSO₄, filtered, and the supernatant concentrated under reduced pressure to afford the crude methyl 5-(2-((N²-acetyl-N⁶-(tert-butoxycarbonyl)lysyl)oxy)acetyl)-2-hydroxybenzoate as a yellowish white solid. This crude product was used in the next step without any purification. Crude methyl 5-(2-((N²-acetyl-N⁶-(tert-butoxycarbonyl)lysyl)oxy)acetyl)-2-hydroxybenzoate was dissolved in TFA/DCM (20 mL) and the resulted solution was stirred under ambient conditions overnight. After completion of the reaction (Boc group removal) confirmed by TLC, the reaction mixture was concentrated under reduced pressure. The residue was dissolved in water and EtOAc (40 mL, 1:1). The water layer was separated, frozen, and then lyophilized to afford Methyl 5-(2-((acetyllysyl)oxy)acetyl)-2-hydroxybenzoate as a light pink colored solid (1.1 g, 85%). ¹H NMR (400 MHz, D₂O), acetic acid as an internal standard, δ (ppm): 8.36 (s, 1H), 8.17-7.71 (m, 1H), 7.04 (d, J=8.8 Hz, 1H), 5.80-5.16 (m, 2H), 4.53 (dd, J=8.9, 5.2 Hz, 1H), 3.94 (s, 3H), 2.99 (t, J=7.6 Hz, 2H), 2.02 (s, 3H), 2.01-1.94 (m, 1H), 1.91-1.77 (m, 1H), 1.76-1.64 (m, 2H), 1.56-1.43 (m, 2H). ¹³C NMR (126 MHz, D₂O) δ (ppm): 192.77, 173.86, 172.72, 169.17, 163.94, 162.35, 134.53, 131.20, 124.82, 117.68, 112.50, 66.60, 52.43, 51.94, 38.67, 29.54, 25.71, 21.47. HRMS (M+H) calculated for C₁₈H₂₅N₂O₇ 380.16, found 381.1643.

### Example 57

### Large Scale HA-Lys-Pt Synthesis: Synthesis of Hyaluronic Acid-pHP-Lys Conjugate

A 250 mL round bottomed flask was charged with hyaluronic acid (75 kDa) sodium salt (1.0 g, 2.5 mmol based on COOH groups per disaccharide unit) in H₂O (40 mL) and 4-(4,6-Dimethoxy-1,3,5-triazin-2-yl)-4-methylmorpholin-4-ium chloride (DMTMM, 2.1 g, 7.5 mmol) was added. The resulted solution was gently stirred for 15 minutes at room temperature. An aqueous solution (10 mL) of methyl 5-(2-((acetyllysyl)oxy)acetyl)-2-hydroxybenzoate (0.95 g, 2.5 mmol) was then added to the reaction mixture and pH of the resulted solution was immediately adjusted to pH 5 using aqueous NaOH solution. The resulted reaction mixture was then gently stirred for 48 hours at 35-37° C. The reaction mixture was then dialyzed (10000 MWC) against NaCl (3×) and ultrapure water (3×) for 48 hours. The resulted solution was then filtered (0.2 μM filter) and lyophilized to afford the title conjugate as a white fluffy solid. The synthetic scheme is shown in FIG. 43. Product was analyzed by ¹H NMR in deuterated water. Degree of substitution (DS) was calculated using the peaks at 3.35 ppm (1H, HA) and 1.60 ppm (2H, Lys). DS was found to be about 20%.

### Example 58

### Large Scale HA-Lys-Pt Synthesis: Synthesis of Hyaluronic Acid-Lys Conjugate

Hyaluronic acid-pHP-Lys conjugate from the above reaction was dissolved in H₂O (40 mL) and photolyzed using a 450 W UV lamp (which emits 40% near UV light) for 4 hours. The small amount was lyophilized and ¹H NMR was performed to check the completion of the photolysis. After completion the photolysis, resulted solution was dialyzed (10000 MWC) against NaCl (3×) and ultrapure water (3×) for 48 hours and filtered (0.2 μM filter) and lyophilized to afford title conjugate as white fluffy solid.

### Example 59

### Large Scale HA-Lys-Pt Synthesis: Synthesis of HA-Lys-Pt Conjugate

Hyaluronic acid-Lys conjugate (100 mg, 0.035 mmol based on Lys) was dissolved in H₂O (12 mL) and stirred in dark. To this solution Pt(NH₃)₂(OH)₂ (11.1 mg, 0.042 mmol) was added in H₂O. The pH of the resulted solution was adjusted to ˜5 using NaOH solution and reaction mixture was gently stirred at 50° C. for 48 hours in the dark. The pH of the solution was maintained around pH 5 throughout the reaction using aqueous NaOH solution. The pH of the reaction mixture dropped significantly within first six hours. During the next 12-24 hours, pH dropped slightly. The pH did not change significantly over the next 18 hours of the reaction. The color of the reaction mixture changed to yellowish green color after 24 hours and intensity of the color increased with time. The change in pH and color indicates the progression of the chelation of cisplatin with lysine linker. After completion, reaction mixture was filtered (0.2 μM filter) and dialyzed (10000 MWC) against NaCl (1× for 6 hours), NaH₂PO₄ (1× for overnight) and ultrapure water (3×) for 24 hours in the dark. The resulted aqueous hyaluronic acid-Lys-Pt conjugate was stored at 4° C. in the dark. Pt substitution (% substitution to Lys residues on hyaluronic acid) was determined by ICP-MS (70%). The degree of Pt loading was determined by drying aqueous hyaluronic acid-Lys-Pt (500 μL) and it was found to be about 6%.

### Example 60

### HA-Homo-Lys-Pt Synthesis: Synthesis and NMR of Methyl 5-(2-((3-((3-((((8aH-fluoren-9-yl)methoxy)carbonyl)amino)-7-((tert butoxycarbonyl)amino) heptanoyl)oxy) acetyl)-2-hydroxybenzoate, 2

To a stirred solution of methyl Fmoc-Homo-Lys-Boc-OH (350 mg, 0.72 mmol) in MeCN (40 mL) was added K₂CO₃ (300 mg, 2.2 mmol). The reaction mixture was stirred for 10 minutes. A solution of 5-bromoacetyl-2-hydroxybenzoic acid methyl ester (198 mg, 0.72 mmol) in MeCN (10 mL) was added dropwise to the reaction mixture. The reaction mixture was stirred under ambient conditions. The progress of the reaction was monitored by TLC. After completion of the reaction, the reaction mixture was filtered, and water (50 mL) and EtOAc (40 mL) were added to the reaction mixture. EtOAc layer was separated and aqueous layer was re-extracted with EtOAc (20 mL×2). The EtOAc fractions were combined, dried over anhydrous MgSO₄, filtered, and the supernatant concentrated under reduced pressure to afford the crude title compound, 2 as a yellowish white solid. Synthetic scheme is shown in FIG. 44. Crude product was purified over silica gel using 2% EtOAc in hexane to afford pure product as a white solid (420 mg, 86%). ¹H NMR (400 MHz, CDCl₃) δ (ppm): 11.36 (s, 1H), 8.47 (d, J=2.0 Hz, 1H), 8.07 (dd, J=8.8, 2.0 Hz, 1H), 7.75 (d, J=7.5 Hz, 2H), 7.63 (dd, J=7.3, 4.0 Hz, 2H), 7.38 (td, J=7.4, 3.9 Hz, 2H), 7.32-7.19 (m, 2H), 7.09 (d, J=8.8 Hz, 1H), 5.86 (d, J=9.4 Hz, 1H), 5.38 (dd, J=101.0, 16.3 Hz, 2H), 4.63 (s, 1H), 4.48-4.32 (m, 2H), 4.24 (t, J=7.2 Hz, 1H), 4.09 (d, J=8.9 Hz, 1H), 4.00 (s, 3H), 3.13 (s, 2H), 2.80 (dd, J=14.6, 5.3 Hz, 1H), 2.68 (dd, J=14.6, 4.7 Hz, 1H), 1.78-1.71 (m, 1H), 1.67 (dt, J=14.5, 7.1 Hz, 2H), 1.53 (dt, J=12.4, 6.7 Hz, 2H), 1.43 (s, 9H), 1.33-1.16 (m, 1H); ¹³C NMR (126 MHz, D₂O) δ (ppm): 190.34, 170.93, 170.01, 166.21, 156.33, 144.26, 141.48, 135.18, 131.17, 127.81, 127.19, 125.81, 125.43, 120.11, 118.80, 112.57, 66.86, 65.93, 53.05, 48.33, 47.46, 39.10, 33.81, 29.82, 28.62, 23.60; HRMS (M+H) calculated for C₃₇H₄₂N₂O₁₀Na 697.2737, found 697.2766. FIG. 44 shows the reaction scheme for examples 60-65.

### Example 61

### HA-Homo-Lys-Pt Synthesis: Synthesis and NMR of Methyl 5-(2-((3-acetamido-7-((tert-butoxycarbonyl)amino)heptanoyl)oxy)acetyl)-2-acetoxybenzoate, 4

A 10 mL round bottomed flask was charged with 2 (410 mg, 0.61 mmol) in dimethylformamide (DMF, 2 mL). While stirring the reaction mixture, piperidine (400 μL) was added and resulted solution was stirred at room temperature for 3 hours. Solvents were removed under reduced pressure and water and EtOAc (10 mL) was added to the flask. EtOAc layer was separated and aqueous layer was re-extracted with EtOAc (5 mL×3). EtOAc fractions were combined, dried over anhydrous MgSO₄, filtered, and the supernatant concentrated under reduced pressure to afford the crude title compound, Methyl 5-(2-((3-amino-7-((tert-butoxycarbonyl)amino) heptanoyl)oxy)acetyl)-2-hydroxybenzoate, 3 as a yellowish brown solid. Crude product was purified over a pad of silica gel using gradient solvent system (EtOAc in hexane, 0 to 100%) to get rid of Fmoc byproducts. Resulted semi-crude crude product was utilized in the next step without further purification. HRMS (M+H) calculated for C₂₂H₃₃N₂O₈ 453.2237, found 453.2256; To a stirred solution of 3 (240 mg, 0.53 mmol) in methylene chloride (DCM, 10 mL) in an ice bath was added triethylamine (148 μL, 1.1 mmol). The reaction mixture was stirred for 10 minutes at 0° C. Acetyl chloride (76 μL, 1.1 mmol) was added dropwise to the reaction mixture. The reaction mixture was stirred for 2 hours at 0° C. The progress of the reaction was monitored by TLC. After completion of the reaction, the reaction mixture was concentrated under reduced pressure followed by addition of water and EtOAc (20 mL) was added to the flask. EtOAc layer was separated and aqueous layer was re-extracted with EtOAc (10 mL×2). The EtOAc fractions were combined, dried over anhydrous MgSO₄, filtered, and the supernatant concentrated under reduced pressure to afford the crude title compound, 4 as a yellowish oily product. Crude product was purified over silica gel using 10% EtOAc in hexane to afford pure product as a yellowish white solid (260 mg, 92%). ¹H NMR (400 MHz, CDCl₃) δ (ppm): 8.57 (d, J=2.1 Hz, 1H), 8.15 (dd, J=8.4, 2.2 Hz, 1H), 6.57 (d, J=8.9 Hz, 1H), 5.42 (dd, J=84.9, 16.6 Hz, 2H), 4.62 (s, 1H), 4.44-4.26 (m, 2H), 3.93 (s, 3H), 3.12 (s, 3H), 2.77 (dd, J=14.7, 5.1 Hz, 1H), 2.66 (dd, J=14.7, 4.3 Hz, 1H), 2.39 (s, 3H), 2.04 (s, 3H), 1.80 (s, 3H), 1.70-1.60 (m, 2H), 1.57-1.47 (m, 2H), 1.44 (s, 9H), 1.43-1.40 (m, 1H); ¹³C NMR (126 MHz, D₂O) δ (ppm): 191.14, 170.33, 169.28, 164.00, 156.33, 155.23, 133.38, 131.86, 131.77, 125.22, 6 6.18, 52.88, 46.12, 38.53, 33.50, 29.87, 28.64, 23.67, 23.56, 21.18; HRMS (M+H) calculated for C₂₆H₃₆N₂O₁₀Na 559.2268, found 559.2263.

### Example 62

### HA-Homo-Lys-Pt Synthesis: Synthesis and NMR of Methyl 5-(2-((3-acetamido-7-aminoheptanoyl)oxy)acetyl)-2-acetoxybenzoate, 5 and Methyl 5-(2-((3-acetamido-7-aminoheptanoyl)oxy)acetyl)-2-hydroxybenzoate, 6

A round bottomed flask (100 mL) was charged with 4 (250 mg, 0.47 mmol) in TFA/DCM (20 mL) and resulted solution was stirred under ambient conditions overnight. After completion of the reaction (Boc group removal) confirmed by TLC, the reaction mixture was concentrated under reduced pressure. The residue was dissolved in water and EtOAc (40 mL, 1:1). The water layer was separated, frozen, and then lyophilized to afford mixture of methyl 5-(2-((3-acetamido-7-aminoheptanoyl)oxy)acetyl)-2-acetoxybenzoate, 5 (major) and methyl 5-(2-((3-acetamido-7-aminoheptanoyl)oxy)acetyl)-2-hydroxybenzoate, 6 (minor) and as a light pink colored oily solid (165 mg, ˜80%). ¹H NMR for 5 (400 MHz, D₂O), acetic acid as an internal standard, δ (ppm): 8.44 (d, J=2.3 Hz, 1H), 8.05 (dd, J=8.8, 2.3 Hz, 1H), 7.08 (d, J=8.8 Hz, 1H), 5.43 (d, J=8.6 Hz, 2H), 4.22 (dq, J=9.0, 4.7 Hz, 1H), 3.96 (s, 3H), 2.96 (t, J=7.5 Hz, 3H), 2.78 (dt, J=15.5, 5.5 Hz, 1H), 2.68-2.63 (m, 1H), 1.97 (s, 3H), 1.71-160 (m, 5H), 1.60-1.49 (m, 2H), 1.47-1.30 (m, 4H); ¹³C NMR (126 MHz, D₂O) δ (ppm): 193.67, 173.58, 172.66, 172.58, 169.68, 164.36, 163.10, 162.81, 134.98, 133.96, 131.67, 125.36, 118.09, 113.00, 66.65, 52.83, 46.18, 39.21, 38.88, 33.00, 26.23, 22.06, 21.86; HRMS (M+H) calculated for C₂₁H₂₈N₂O₈ 436.18, found 437.20.

### Example 63

### HA-Homo-Lys-Pt Synthesis: Synthesis and NMR of Hyaluronic Acid-pHP-Homo-Lys Conjugate

A 100 mL round bottomed flask was charged with hyaluronic acid (75 kDa) sodium salt (140 mg, 0.35 mmol based on COOH groups per disaccharide unit) in H₂O (5 mL) and 4-(4,6-Dimethoxy-1,3,5-triazin-2-yl)-4-methylmorpholin-4-ium chloride (DMTMM, 289 mg, 1.04 mmol) was added. The resulted solution was gently stirred for 15 minutes at room temperature. An aqueous solution (2 mL) of methyl 5-(2-((3-acetamido-7-aminoheptanoyl)oxy)acetyl)-2-acetoxybenzoate, 5 (major) and methyl 5-(2-((3-acetamido-7-aminoheptanoyl)oxy)acetyl)-2-hydroxybenzoate, 6 (140 mg, 0.32 mmol) was then added to the reaction mixture and pH of the resulted solution was immediately adjusted to 5 using aqueous NaOH solution. The resulted reaction mixture was gently stirred for 48 hours at 37° C. The reaction mixture was dialyzed (10000 MWC) against NaCl (3×) and ultrapure water (3×) for 48 hours. The resulted solution was then filtered (0.2 μM filter) and lyophilized to afford the title conjugate as a white fluffy solid. Product was analyzed by ¹H NMR in deuterated water. Degree of substitution (DS) was calculated using the peaks at 3.35 ppm (1H, HA) and 5.60 ppm (2H, p-acetoxyphenacyl CH₂). DS was found to be ˜20%.

### Example 64

### HA-Homo-Lys-Pt Synthesis: Synthesis of Hyaluronic Acid-Homo-Lys Conjugate

Hyaluronic acid-pHP-Homo-Lys conjugate from the above reaction was dissolved in H₂O (20 mL) and photolyzed using a 450 W UV lamp (which emits 40% near UV light) for 2 hours. The small amount was lyophilized and ¹H NMR was performed to check the completion of the photolysis. After completion the photolysis, resulted solution was dialyzed (10000 MWC) against NaCl (3×) and ultrapure water (3×) for 48 h and filtered (0.2 μM filter) and lyophilized to afford title conjugate as white fluffy solid.

### Example 65

HA-Homo-Lys-Pt Synthesis: Synthesis and Characterization of Hyaluronic acid-Homo-Lys-Pt Conjugate

Hyaluronic acid-homo-Lys conjugate (125 mg, 0.041 mmol based on Lys) was dissolved in H₂O (15 mL) and stirred in dark. To this solution Pt(NH₃)₂(OH)₂ (13.1 mg, 0.050 mmol) was added in H₂O. The pH of the resulted solution was adjusted to ˜5 using NaOH solution and reaction mixture was gently stirred at 50° C. for 48 hours in the dark. The pH of the solution was maintained around 5 throughout the reaction using aqueous NaOH solution. The pH of the reaction mixture dropped significantly within the first 24 hours. Over the next 24 hours, pH dropped slightly. The color of the reaction mixture changed to yellowish green color overnight and intensity of the color increased with time. The color change indicates the chelation of cisplatin with the homo lysine linker. The rate of change and chelation of the homo lysine linker was substantially more rapid than the acetyl lysine linker. After completion, the reaction mixture was filtered (0.2 μM filter) and dialyzed (10000 MWC) against NaCl (1× for 6 h), NaH₂PO₄ (1× for overnight) and ultrapure water (3×) for 24 hours in the dark. The resulted aqueous hyaluronic acid-homo-Lys-Pt conjugate was stored at 4° C. in the dark. Pt substitution (% substitution to homo-Lys residues on hyaluronic acid) was determined by ICP-MS (˜65%). The degree of Pt loading was determined by drying aqueous hyaluronic acid-homo-Lys-Pt (500 μL) and it was found to be ˜6%.

### Example 66

### Molecular Modeling of Cisplatin Chelates with Acetyl Lysine and Acetyl Homolysine

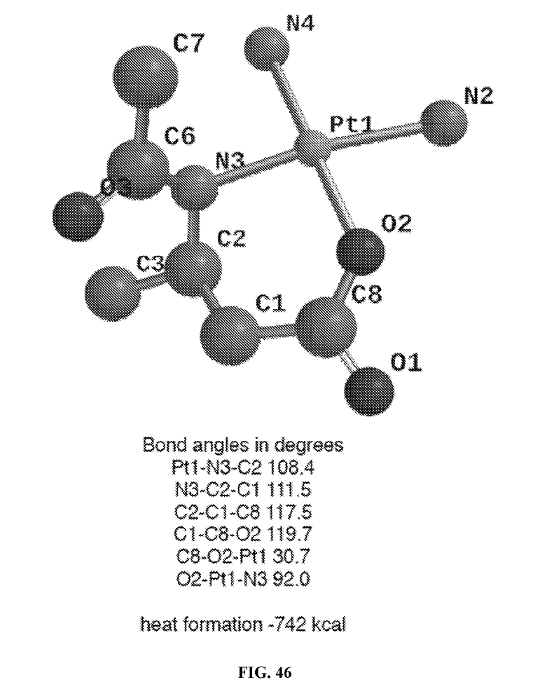

It is a usual assumption that 5 member rings are more stable than 6 member rings, which would result in potentially faster chelation and more slow release. Although, both 6 and 5 member rings generally have similar enthalpies in most organics, the 6 member rings have more degrees of freedom, so the 5 member ring is preferred. However, the Pt in the ring conforms to about 90 degs, which could alter the bond conformation of the ring compared to a similar carbon ring. Spartan'14 molecular modeling of the 5 member ring (FIG. 45) and 6 member ring (FIG. 46) gave a predicted enthalpy of formation of about 40 kcal less for the 6 member ring than the 5 member.

### Example 67

### Stability Testing

A series of formulation screening experiments were conducted to evaluate the stability of HA-Pt in the presence of different excipients (e.g., NaCl, mannitol, and excess HA). The concentration of NaCl varies from 0-9 mg/mL. Concentration of HA-Pt was between 9.6 to 10 mg/mL on cisplatin basis. pH values varies from 6.5 to 7.4. Storage temperature varied from 4 to 40° C. Concentration of mannitol varied from 0 to 290 mOsm. Concentration of excess HA (35 kDa) varied from 0 to 33.6 mg/mL. Samples were monitored for the formation of precipitate and coloration over time. Representative examples are listed below as sub-examples 67-1 to 67-156.

### Example 67-1

HA-Pt (9.6 mg/mL on cisplatin basis) was mixed with NaCl (1.95 mg/mL) at pH 6.5 and incubated at 4° C. Yellowish precipitate was observed on day 5.

### Example 67-2

HA-Pt (9.6 mg/mL) was mixed with NaCl (1.95 mg/mL) at pH 6.5 and incubated at 25° C. Yellowish precipitate was observed on day 6.

### Example 67-3

HA-Pt (9.6 mg/mL) was mixed with NaCl (1.95 mg/mL) at pH 6.5 and incubated at 40° C. No precipitate was observed within 5 months.

### Example 67-4

HA-Pt (9.6 mg/mL) was mixed with NaCl (1.95 mg/mL) at pH 7 and incubated at 4° C. Yellowish precipitate was observed on day 3.

### Example 67-5

HA-Pt (9.6 mg/mL) was mixed with NaCl (1.95 mg/mL) at pH 7 and incubated at 25° C. Yellowish precipitate was observed on day 6.

### Example 67-6

HA-Pt (9.6 mg/mL) was mixed with NaCl (1.95 mg/mL) at pH 7 and incubated at 40° C. No precipitate was observed within 5 months.

### Example 67-7

HA-Pt (9.6 mg/mL) was mixed with NaCl (1.95 mg/mL) at pH 7.4 and incubated at 4° C. Yellowish precipitate was observed on day 5.

### Example 67-8

HA-Pt (9.6 mg/mL) was mixed with NaCl (1.95 mg/mL) at pH 7.4 and incubated at 25° C. Yellowish precipitate was observed on day 6.

### Example 67-9

HA-Pt (9.6 mg/mL) was mixed with NaCl (1.95 mg/mL) at pH 7.4 and incubated at 40° C. No precipitate was observed within 5 months.

### Example 67-10

HA-Pt (9.6 mg/mL) was mixed with NaCl (2.9 mg/mL) at pH 6.5 and incubated at 4° C. No precipitate was observed within 5 months.

### Example 67-11

HA-Pt (9.6 mg/mL) was mixed with NaCl (2.9 mg/mL) at pH 6.5 and incubated at 25° C. No precipitate was observed within 5 months.

### Example 67-12

HA-Pt (9.6 mg/mL) was mixed with NaCl (2.9 mg/mL) at pH 6.5 and incubated at 40° C. No precipitate was observed within 5 months.

### Example 67-13

HA-Pt (9.6 mg/mL) was mixed with NaCl (2.9 mg/mL) at pH 7 and incubated at 4° C. Yellowish precipitate was observed on day 2.

### Example 67-14

HA-Pt (9.6 mg/mL) was mixed with NaCl (2.9 mg/mL) at pH 7 and incubated at 25° C. Yellowish precipitate was observed on day 2.

### Example 67-15

HA-Pt (9.6 mg/mL) was mixed with NaCl (2.9 mg/mL) at pH 7 and incubated at 40° C. Yellowish precipitate was observed on day 1.

### Example 67-16

HA-Pt (9.6 mg/mL) was mixed with NaCl (2.9 mg/mL) at pH 7.4 and incubated at 4° C. No precipitate was observed within 5 months.

### Example 67-17

HA-Pt (9.6 mg/mL) was mixed with NaCl (2.9 mg/mL) at pH 7.4 and incubated at 25° C. No precipitate was observed within 5 months.

### Example 67-18

HA-Pt (9.6 mg/mL) was mixed with NaCl (2.9 mg/mL) at pH 7.4 and incubated at 40° C. No precipitate was observed within 5 months.

### Example 76-19

HA-Pt (9.6 mg/mL) was mixed with NaCl (3.9 mg/mL) at pH 6.5 and incubated at 4° C. Yellowish precipitate was observed on day 2.

### Example 67-20

HA-Pt (9.6 mg/mL) was mixed with NaCl (3.9 mg/mL) at pH 6.5 and incubated at 25° C. Yellowish precipitate was observed on day 2.

### Example 67-21

HA-Pt (9.6 mg/mL) was mixed with NaCl (3.9 mg/mL) at pH 6.5 and incubated at 40° C. Yellowish precipitate was observed on day 1.

### Example 67-22

HA-Pt (9.6 mg/mL) was mixed with NaCl (3.9 mg/mL) at pH 7 and incubated at 4° C. Yellowish precipitate was observed on day 2.

### Example 67-23

HA-Pt (9.6 mg/mL) was mixed with NaCl (3.9 mg/mL) at pH 7 and incubated at 25° C. Yellowish precipitate was observed on day 1.

### Example 67-24

HA-Pt (9.6 mg/mL) was mixed with NaCl (3.9 mg/mL) at pH 7 and incubated at 40° C. Yellowish precipitate was observed on day 1.

### Example 67-25

HA-Pt (9.6 mg/mL) was mixed with NaCl (3.9 mg/mL) at pH 7.4 and incubated at 4° C. Yellowish precipitate was observed on day 2.

### Example 67-26

HA-Pt (9.6 mg/mL) was mixed with NaCl (3.9 mg/mL) at pH 7.4 and incubated at 25° C. Yellowish precipitate was observed on day 2.

### Example 67-27

HA-Pt (9.6 mg/mL) was mixed with NaCl (3.9 mg/mL) at pH 7.4 and incubated at 40° C. Yellowish precipitate was observed on day 1.

### Example 67-28

HA-Pt (10 mg/mL) was mixed with 35 kDa HA (5.6 mg/mL) at pH 6.5 and incubated at 4° C. No precipitate was observed within 5 months.

### Example 67-29

HA-Pt (10 mg/mL) was mixed with 35 kDa HA (5.6 mg/mL) at pH 7 and incubated at 4° C. No precipitate was observed within 5 months.

### Example 67-30

HA-Pt (10 mg/mL) was mixed with 35 kDa HA (5.6 mg/mL) at pH 7.4 and incubated at 4° C. No precipitate was observed within 5 months.

### Example 67-31

HA-Pt (10 mg/mL) was mixed with 35 kDa HA (16.4 mg/mL) at pH 6.5 and incubated at 4° C. No precipitate was observed within 5 months.

### Example 67-32

HA-Pt (10 mg/mL) was mixed with 35 kDa HA (16.4 mg/mL) at pH 7 and incubated at 4° C. No precipitate was observed within 5 months.

### Example 67-33

HA-Pt (10 mg/mL) was mixed with 35 kDa HA (16.4 mg/mL) at pH 7.4 and incubated at 4° C. No precipitate was observed within 5 months.

### Example 67-34

HA-Pt (10 mg/mL) was mixed with 35 kDa HA (33.6 mg/mL) at pH 6.5 and incubated at 4° C. No precipitate was observed within 5 months.

### Example 67-35

HA-Pt (10 mg/mL) was mixed with 35 kDa HA (33.6 mg/mL) at pH 7 and incubated at 4° C. No precipitate was observed within 5 months.

### Example 67-36

HA-Pt (10 mg/mL) was mixed with 35 kDa HA (33.6 mg/mL) at pH 7.4 and incubated at 4° C. No precipitate was observed within 5 months.

### Example 67-37

HA-Pt (10 mg/mL) was mixed with NaCl (0.98 mg/mL) at pH 6.5 and incubated at 4° C. Yellowish precipitate was observed on day 5.

### Example 67-38

HA-Pt (10 mg/mL) was mixed with NaCl (0.98 mg/mL) at pH 7 and incubated at 4° C. Yellowish precipitate was observed on day 11.

### Example 67-39

HA-Pt (10 mg/mL) was mixed with NaCl (0.98 mg/mL) at pH 7.4 and incubated at 4° C. Yellowish precipitate was observed on day 11.

### Example 67-40

HA-Pt (10 mg/mL) was mixed with sodium citrate (1.88% wt) at pH 6.5 and incubated at 4° C. Yellowish precipitate was observed on day 3.

### Example 67-41

HA-Pt (10 mg/mL) was mixed with sodium citrate (1% wt) at pH 6.5 and incubated at 4° C. Yellowish precipitate was observed on day 4.

### Example 67-42

HA-Pt (10 mg/mL) was mixed with 35 kDa HA (5.6 mg/mL) at pH 6.5 and incubated at 25° C. No precipitate was observed within 5 months.

### Example 67-43

HA-Pt (10 mg/mL) was mixed with 35 kDa HA (5.6 mg/mL) at pH 7 and incubated at 25° C. No precipitate was observed within 5 months.

### Example 67-44

HA-Pt (10 mg/mL) was mixed with 35 kDa HA (5.6 mg/mL) at pH 7.4 and incubated at 25° C. No precipitate was observed within 5 months.

### Example 67-45

HA-Pt (10 mg/mL) was mixed with 35 kDa HA (16.4 mg/mL) at pH 6.5 and incubated at 25° C. No precipitate was observed within 5 months.

### Example 67-46

HA-Pt (10 mg/mL) was mixed with 35 kDa HA (16.4 mg/mL) at pH 7 and incubated at 25° C. No precipitate was observed within 5 months.

### Example 67-47

HA-Pt (10 mg/mL) was mixed with 35 kDa HA (16.4 mg/mL) at pH 7.4 and incubated at 25° C. No precipitate was observed within 5 months.

### Example 67-48

HA-Pt (10 mg/mL) was mixed with 35 kDa HA (33.6 mg/mL) at pH 6.5 and incubated at 25° C. No precipitate was observed within 5 months.

### Example 67-49

HA-Pt (10 mg/mL) was mixed with 35 kDa HA (33.6 mg/mL) at pH 7 and incubated at 25° C. No precipitate was observed within 5 months.

### Example 67-50

HA-Pt (10 mg/mL) was mixed with 35 kDa HA (33.6 mg/mL) at pH 7.4 and incubated at 25° C. No precipitate was observed within 5 months.

### Example 67-51

HA-Pt (10 mg/mL) was mixed with NaCl (0.98 mg/mL) at pH 6.5 and incubated at 25° C. No precipitate was observed within 5 months.

### Example 67-52

HA-Pt (10 mg/mL) was mixed with NaCl (0.98 mg/mL) at pH 7 and incubated at 25° C. No precipitate was observed within 5 months.

### Example 67-53

HA-Pt (10 mg/mL) was mixed with NaCl (0.98 mg/mL) at pH 7.4 and incubated at 25° C. No precipitate was observed within 5 months.

### Example 67-54

HA-Pt (10 mg/mL) was mixed with sodium citrate (1.88% wt) at pH 6.5 and incubated at 25° C. Yellowish precipitate was observed on day 1.

### Example 67-55

HA-Pt (10 mg/mL) was mixed with sodium citrate (1% wt) at pH 6.5 and incubated at 25° C. Yellowish precipitate was observed on day 3.

### Example 67-56

HA-Pt (10 mg/mL) was mixed with 35 kDa HA (5.6 mg/mL) at pH 6.5 and incubated at 40° C. No precipitate was observed within 5 months. Coloration (e.g. dark green) was observed since week 6.

### Example 67-57

HA-Pt (10 mg/mL) was mixed with 35 kDa HA (5.6 mg/mL) at pH 7 and incubated at 40° C. No precipitate was observed within 5 months. Coloration (e.g. dark green) was observed since week 6.

### Example 67-58

HA-Pt (10 mg/mL) was mixed with 35 kDa HA (5.6 mg/mL) at pH 7.4 and incubated at 40° C. No precipitate was observed within 5 months. Coloration (e.g. dark green) was observed since week 6.

### Example 67-59

HA-Pt (10 mg/mL) was mixed with 35 kDa HA (16.4 mg/mL) at pH 6.5 and incubated at 40° C. No precipitate was observed within 5 months. Coloration (e.g. dark green) was observed since week 6.

### Example 67-60

HA-Pt (10 mg/mL) was mixed with 35 kDa HA (16.4 mg/mL) at pH 7 and incubated at 40° C. No precipitate was observed within 5 months. Coloration (e.g. dark green) was observed since week 6.

### Example 67-61

HA-Pt (10 mg/mL) was mixed with 35 kDa HA (16.4 mg/mL) at pH 7.4 and incubated at 40° C. No precipitate was observed within 5 months. Coloration (e.g. dark green) was observed since week 6.

### Example 67-62

HA-Pt (10 mg/mL) was mixed with 35 kDa HA (33.6 mg/mL) at pH 6.5 and incubated at 40° C. No precipitate was observed within 5 months. Coloration (e.g. dark green) was observed since week 6.

### Example 67-63

HA-Pt (10 mg/mL) was mixed with 35 kDa HA (33.6 mg/mL) at pH 7 and incubated at 40° C. No precipitate was observed within 5 months. Coloration (e.g. dark green) was observed since week 6.

### Example 67-64

HA-Pt (10 mg/mL) was mixed with 35 kDa HA (33.6 mg/mL) at pH 7.4 and incubated at 40° C. No precipitate was observed within 5 months. Coloration (e.g. dark green) was observed since week 6.

### Example 67-65

HA-Pt (10 mg/mL) was mixed with NaCl (0.98 mg/mL) at pH 6.5 and incubated at 40° C. No precipitate was observed within 5 months. Coloration (e.g. dark green) was observed since week 6.

### Example 67-66

HA-Pt (10 mg/mL) was mixed with NaCl (0.98 mg/mL) at pH 7 and incubated at 40° C. No precipitate was observed within 5 months. Coloration (e.g. dark green) was observed since week 6.

### Example 67-67

HA-Pt (10 mg/mL) was mixed with NaCl (0.98 mg/mL) at pH 7.4 and incubated at 40° C. No precipitate was observed within 5 months. Coloration (e.g. dark green) was observed since week 6.

### Example 67-68

HA-Pt (10 mg/mL) was mixed with sodium citrate (1.88% wt) at pH 6.5 and incubated at 40° C. No precipitate was observed within 5 months.

### Example 67-69

HA-Pt (10 mg/mL) was mixed with sodium citrate (1% wt) at pH 6.5 and incubated at 40° C. No precipitate was observed within 5 months.

### Example 67-70

HA-Pt (10 mg/mL) was mixed with NaCl (0.03 mg/mL) at pH 6.5 and incubated at 4° C. No precipitate was observed within 5 months.

### Example 67-71

HA-Pt (10 mg/mL) was mixed with NaCl (0.03 mg/mL) and mannitol (290 mOsm) at pH 6.5 and incubated at 4° C. No precipitate was observed within 5 months.

### Example 67-72

HA-Pt (10 mg/mL) was mixed with NaCl (0.03 mg/mL) and mannitol (145 mOsm) at pH 6.5 and incubated at 4° C. No precipitate was observed within 5 months.

### Example 67-73

HA-Pt (10 mg/mL) was mixed with NaCl (0.06 mg/mL) at pH 6.5 and incubated at 4° C. No precipitate was observed within 5 months.

### Example 67-74

HA-Pt (10 mg/mL) was mixed with NaCl (0.06 mg/mL) and mannitol (290 mOsm) at pH 6.5 and incubated at 4° C. No precipitate was observed within 5 months.

### Example 67-75

HA-Pt (10 mg/mL) was mixed with NaCl (0.06 mg/mL) and mannitol (145 mOsm) at pH 6.5 and incubated at 4° C. No precipitate was observed within 5 months.

### Example 67-76

HA-Pt (10 mg/mL) was mixed with NaCl (0.12 mg/mL) at pH 6.5 and incubated at 4° C. No precipitate was observed within 5 months.

### Example 67-77

HA-Pt (10 mg/mL) was mixed with NaCl (0.12 mg/mL) and mannitol (290 mOsm) at pH 6.5 and incubated at 4° C. No precipitate was observed within 5 months.

### Example 67-78

HA-Pt (10 mg/mL) was mixed with NaCl (0.12 mg/mL) and mannitol (145 mOsm) at pH 6.5 and incubated at 4° C. No precipitate was observed within 5 months.

### Example 67-79

HA-Pt (10 mg/mL) was mixed with NaCl (0.03 mg/mL) at pH 6.5 and incubated at 25° C. No precipitate was observed within 5 months.

### Example 67-80

HA-Pt (10 mg/mL) was mixed with NaCl (0.03 mg/mL) and mannitol (290 mOsm) at pH 6.5 and incubated at 25° C. No precipitate was observed within 5 months.

### Example 67-81

HA-Pt (10 mg/mL) was mixed with NaCl (0.03 mg/mL) and mannitol (145 mOsm) at pH 6.5 and incubated at 25° C. No precipitate was observed within 5 months.

### Example 67-82

HA-Pt (10 mg/mL) was mixed with NaCl (0.06 mg/mL) at pH 6.5 and incubated at 25° C. No precipitate was observed within 5 months.

### Example 67-83

HA-Pt (10 mg/mL) was mixed with NaCl (0.06 mg/mL) and mannitol (290 mOsm) at pH 6.5 and incubated at 25° C. No precipitate was observed within 5 months.

### Example 67-84

HA-Pt (10 mg/mL) was mixed with NaCl (0.06 mg/mL) and mannitol (145 mOsm) at pH 6.5 and incubated at 25° C. No precipitate was observed within 5 months.

### Example 67-85

HA-Pt (10 mg/mL) was mixed with NaCl (0.12 mg/mL) at pH 6.5 and incubated at 25° C. No precipitate was observed within 5 months.

### Example 67-86

HA-Pt (10 mg/mL) was mixed with NaCl (0.12 mg/mL) and mannitol (290 mOsm) at pH 6.5 and incubated at 25° C. No precipitate was observed within 5 months.

### Example 67-87

HA-Pt (10 mg/mL) was mixed with NaCl (0.12 mg/mL) and mannitol (145 mOsm) at pH 6.5 and incubated at 25° C. No precipitate was observed within 5 months.

### Example 67-88

HA-Pt (10 mg/mL) was mixed with NaCl (0.03 mg/mL) at pH 6.5 and incubated at 40° C. No precipitate was observed within 5 months. Coloration (e.g. dark green) was observed since week 6.

### Example 67-89

HA-Pt (10 mg/mL) was mixed with NaCl (0.03 mg/mL) and mannitol (290 mOsm) at pH 6.5 and incubated at 40° C. No precipitate was observed within 5 months. Coloration (e.g. dark green) was observed since week 6.

### Example 67-90

HA-Pt (10 mg/mL) was mixed with NaCl (0.03 mg/mL) and mannitol (145 mOsm) at pH 6.5 and incubated at 40° C. No precipitate was observed within 5 months. Coloration (e.g. dark green) was observed since week 6.

### Example 67-91

HA-Pt (10 mg/mL) was mixed with NaCl (0.06 mg/mL) at pH 6.5 and incubated at 40° C. No precipitate was observed within 5 months. Coloration (e.g. dark green) was observed since week 6.

### Example 67-92

HA-Pt (10 mg/mL) was mixed with NaCl (0.06 mg/mL) and mannitol (290 mOsm) at pH 6.5 and incubated at 40° C. No precipitate was observed within 5 months. Coloration (e.g. dark green) was observed since week 6.

### Example 67-93

HA-Pt (10 mg/mL) was mixed with NaCl (0.06 mg/mL) and mannitol (145 mOsm) at pH 6.5 and incubated at 40° C. No precipitate was observed within 5 months. Coloration (e.g. dark green) was observed since week 6.

### Example 67-94

HA-Pt (10 mg/mL) was mixed with NaCl (0.12 mg/mL) at pH 6.5 and incubated at 40° C. No precipitate was observed within 5 months. Coloration (e.g. dark green) was observed since week 6.

### Example 67-95

HA-Pt (10 mg/mL) was mixed with NaCl (0.12 mg/mL) and mannitol (290 mOsm) at pH 6.5 and incubated at 40° C. No precipitate was observed within 5 months. Coloration (e.g. dark green) was observed since week 6.

### Example 67-96

HA-Pt (10 mg/mL) was mixed with NaCl (0.12 mg/mL) and mannitol (145 mOsm) at pH 6.5 and incubated at 40° C. No precipitate was observed within 5 months. Coloration (e.g. dark green) was observed since week 6.

### Example 67-97

HA-Pt (10 mg/mL) was mixed with NaCl (0.25 mg/mL) and mannitol (290 mOsm) at pH 7.4 and incubated at 4° C. No precipitate was observed within 4.5 months.

### Example 67-98

HA-Pt (10 mg/mL) was mixed with NaCl (0.5 mg/mL) and mannitol (290 mOsm) at pH 7.4 and incubated at 4° C. No precipitate was observed within 4.5 months.

### Example 67-99

HA-Pt (10 mg/mL) was mixed with NaCl (0.75 mg/mL) and mannitol (290 mOsm) at pH 7.4 and incubated at 4° C. No precipitate was observed within 4.5 months.

### Example 67-100

HA-Pt (10 mg/mL) was mixed with NaCl (1 mg/mL) and mannitol (290 mOsm) at pH 7.4 and incubated at 4° C. No precipitate was observed within 4.5 months.

### Example 67-101

HA-Pt (10 mg/mL) was mixed with NaCl (1.25 mg/mL) and mannitol (290 mOsm) at pH 7.4 and incubated at 4° C. No precipitate was observed within 4.5 months.

### Example 67-102

HA-Pt (10 mg/mL) was mixed with NaCl (1.5 mg/mL) and mannitol (290 mOsm) at pH 7.4 and incubated at 4° C. Yellowish precipitate was observed on day 30.

### Example 67-103

HA-Pt (10 mg/mL) was mixed with NaCl (1.75 mg/mL) and mannitol (290 mOsm) at pH 7.4 and incubated at 4° C. Yellowish precipitate was observed on day 25.

### Example 67-104

HA-Pt (10 mg/mL) was mixed with NaCl (2 mg/mL) and mannitol (290 mOsm) at pH 7.4 and incubated at 4° C. Yellowish precipitate was observed on day 20.

### Example 67-105

HA-Pt (10 mg/mL) was mixed with NaCl (2.25 mg/mL) and mannitol (290 mOsm) at pH 7.4 and incubated at 4° C. Yellowish precipitate was observed on day 20.

### Example 67-106

HA-Pt (10 mg/mL) was mixed with NaCl (2.5 mg/mL) and mannitol (290 mOsm) at pH 7.4 and incubated at 4° C. Yellowish precipitate was observed on day 19.

### Example 67-107

HA-Pt (10 mg/mL) was mixed with NaCl (2.75 mg/mL) and mannitol (290 mOsm) at pH 7.4 and incubated at 4° C. Yellowish precipitate was observed on day 19.

### Example 67-108

HA-Pt (10 mg/mL) was mixed with NaCl (3 mg/mL) and mannitol (290 mOsm) at pH 7.4 and incubated at 4° C. Yellowish precipitate was observed on day 19.

### Example 67-109

HA-Pt (10 mg/mL) was mixed with NaCl (0.25 mg/mL) and mannitol (290 mOsm) at pH 7.4 and incubated at 25° C. No precipitate was observed within 4.5 months.

### Example 67-110

HA-Pt (10 mg/mL) was mixed with NaCl (0.5 mg/mL) and mannitol (290 mOsm) at pH 7.4 and incubated at 25° C. No precipitate was observed within 4.5 months.

### Example 67-111

HA-Pt (10 mg/mL) was mixed with NaCl (0.75 mg/mL) and mannitol (290 mOsm) at pH 7.4 and incubated at 25° C. No precipitate was observed within 4.5 months.

### Example 67-112

HA-Pt (10 mg/mL) was mixed with NaCl (1 mg/mL) and mannitol (290 mOsm) at pH 7.4 and incubated at 25° C. No precipitate was observed within 4.5 months.

### Example 67-113

HA-Pt (10 mg/mL) was mixed with NaCl (1.25 mg/mL) and mannitol (290 mOsm) at pH 7.4 and incubated at 25° C. No precipitate was observed within 4.5 months.

### Example 67-114

HA-Pt (10 mg/mL) was mixed with NaCl (1.5 mg/mL) and mannitol (290 mOsm) at pH 7.4 and incubated at 25° C. No precipitate was observed within 4.5 months.

### Example 67-115

HA-Pt (10 mg/mL) was mixed with NaCl (1.75 mg/mL) and mannitol (290 mOsm) at pH 7.4 and incubated at 25° C. No precipitate was observed within 4.5 months.

### Example 67-116

HA-Pt (10 mg/mL) was mixed with NaCl (2 mg/mL) and mannitol (290 mOsm) at pH 7.4 and incubated at 25° C. No precipitate was observed within 4.5 months.

### Example 67-117

HA-Pt (10 mg/mL) was mixed with NaCl (2.25 mg/mL) and mannitol (290 mOsm) at pH 7.4 and incubated at 25° C. No precipitate was observed within 4.5 months.

### Example 67-118

HA-Pt (10 mg/mL) was mixed with NaCl (2.5 mg/mL) and mannitol (290 mOsm) at pH 7.4 and incubated at 25° C. No precipitate was observed within 4.5 months.

### Example 67-119

HA-Pt (10 mg/mL) was mixed with NaCl (2.75 mg/mL) and mannitol (290 mOsm) at pH 7.4 and incubated at 25° C. No precipitate was observed within 4.5 months.

### Example 67-120

HA-Pt (10 mg/mL) was mixed with NaCl (3 mg/mL) and mannitol (290 mOsm) at pH 7.4 and incubated at 25° C. No precipitate was observed within 4.5 months.

### Example 67-121

HA-Pt (10 mg/mL) was mixed with NaCl (0.25 mg/mL) and mannitol (290 mOsm) at pH 7.4 and incubated at 40° C. No precipitate was observed within 4.5 months.

Coloration (e.g. dark green) was observed since week 6.

### Example 67-122

HA-Pt (10 mg/mL) was mixed with NaCl (0.5 mg/mL) and mannitol (290 mOsm) at pH 7.4 and incubated at 40° C. No precipitate was observed within 4.5 months. Coloration (e.g. dark green) was observed since week 6.

### Example 67-123

HA-Pt (10 mg/mL) was mixed with NaCl (0.75 mg/mL) and mannitol (290 mOsm) at pH 7.4 and incubated at 40° C. No precipitate was observed within 4.5 months. Coloration (e.g. dark green) was observed since week 6.

### Example 67-124

HA-Pt (10 mg/mL) was mixed with NaCl (1 mg/mL) and mannitol (290 mOsm) at pH 7.4 and incubated at 40° C. No precipitate was observed within 4.5 months. Coloration (e.g. dark green) was observed since week 6.

### Example 67-125

HA-Pt (10 mg/mL) was mixed with NaCl (1.25 mg/mL) and mannitol (290 mOsm) at pH 7.4 and incubated at 25° C. No precipitate was observed within 4.5 months.

### Example 67-126

HA-Pt (10 mg/mL) was mixed with NaCl (1.5 mg/mL) and mannitol (290 mOsm) at pH 7.4 and incubated at 40° C. No precipitate was observed within 4.5 months. Coloration (e.g. dark green) was observed since week 6.

### Example 67-127

HA-Pt (10 mg/mL) was mixed with NaCl (1.75 mg/mL) and mannitol (290 mOsm) at pH 7.4 and incubated at 40° C. No precipitate was observed within 4.5 months. Coloration (e.g. dark green) was observed since week 6.

### Example 67-128

HA-Pt (10 mg/mL) was mixed with NaCl (2 mg/mL) and mannitol (290 mOsm) at pH 7.4 and incubated at 40° C. No precipitate was observed within 4.5 months. Coloration (e.g. dark green) was observed since week 6.

### Example 67-129

HA-Pt (10 mg/mL) was mixed with NaCl (2.25 mg/mL) and mannitol (290 mOsm) at pH 7.4 and incubated at 40° C. No precipitate was observed within 4.5 months.

Coloration (e.g. dark green) was observed since week 6.

### Example 67-130

HA-Pt (10 mg/mL) was mixed with NaCl (2.5 mg/mL) and mannitol (290 mOsm) at pH 7.4 and incubated at 40° C. No precipitate was observed within 4.5 months. Coloration (e.g. dark green) was observed since week 6.

### Example 67-131

HA-Pt (10 mg/mL) was mixed with NaCl (2.75 mg/mL) and mannitol (290 mOsm) at pH 7.4 and incubated at 40° C. No precipitate was observed within 4.5 months. Coloration (e.g. dark green) was observed since week 6.

### Example 67-132

HA-Pt (10 mg/mL) was mixed with NaCl (3 mg/mL) and mannitol (290 mOsm) at pH 7.4 and incubated at 40° C. Yellowish precipitate was observed on day 66. Coloration (e.g. dark green) was observed since week 4.

### Example 67-133

HA-Pt (10 mg/mL) was mixed with NaCl (3.5 mg/mL) and mannitol (290 mOsm) at pH 7.4 and incubated at 4° C. Yellowish precipitate was observed on day 6.

### Example 67-134

HA-Pt (10 mg/mL) was mixed with NaCl (4 mg/mL) and mannitol (290 mOsm) at pH 7.4 and incubated at 4° C. Yellowish precipitate was observed on day 5.

### Example 67-135

HA-Pt (10 mg/mL) was mixed with NaCl (4.5 mg/mL) and mannitol (290 mOsm) at pH 7.4 and incubated at 4° C. Yellowish precipitate was observed on day 3.

### Example 67-136

HA-Pt (10 mg/mL) was mixed with NaCl (5 mg/mL) and mannitol (290 mOsm) at pH 7.4 and incubated at 4° C. Yellowish precipitate was observed on day 3.

### Example 67-137

HA-Pt (10 mg/mL) was mixed with NaCl (5.5 mg/mL) and mannitol (290 mOsm) at pH 7.4 and incubated at 4° C. Yellowish precipitate was observed on day 3.

### Example 67-138

HA-Pt (10 mg/mL) was mixed with NaCl (6 mg/mL) and mannitol (290 mOsm) at pH 7.4 and incubated at 4° C. Yellowish precipitate was observed on day 3.

### Example 67-139

HA-Pt (10 mg/mL) was mixed with NaCl (6.5 mg/mL) and mannitol (290 mOsm) at pH 7.4 and incubated at 4° C. Yellowish precipitate was observed on day 3.

### Example 67-140

HA-Pt (10 mg/mL) was mixed with NaCl (7 mg/mL) and mannitol (290 mOsm) at pH 7.4 and incubated at 4° C. Yellowish precipitate was observed on day 3.

### Example 67-141

HA-Pt (10 mg/mL) was mixed with NaCl (7.5 mg/mL) and mannitol (290 mOsm) at pH 7.4 and incubated at 4° C. Yellowish precipitate was observed on day 3.

### Example 67-142

HA-Pt (10 mg/mL) was mixed with NaCl (8 mg/mL) and mannitol (290 mOsm) at pH 7.4 and incubated at 4° C. Yellowish precipitate was observed on day 3.

### Example 67-143

HA-Pt (10 mg/mL) was mixed with NaCl (8.5 mg/mL) and mannitol (290 mOsm) at pH 7.4 and incubated at 4° C. Yellowish precipitate was observed on day 3.

### Example 67-144

HA-Pt (10 mg/mL) was mixed with NaCl (9 mg/mL) and mannitol (290 mOsm) at pH 7.4 and incubated at 4° C. Yellowish precipitate was observed on day 3.

### Example 67-145

HA-Pt (10 mg/mL) was mixed with NaCl (3.5 mg/mL) and mannitol (290 mOsm) at pH 7.4 and incubated at 25° C. No precipitate was observed within 4 months.

### Example 67-146

HA-Pt (10 mg/mL) was mixed with NaCl (4 mg/mL) and mannitol (290 mOsm) at pH 7.4 and incubated at 25° C. No precipitate was observed within 4 months.

### Example 67-147

HA-Pt (10 mg/mL) was mixed with NaCl (4.5 mg/mL) and mannitol (290 mOsm) at pH 7.4 and incubated at 25° C. Yellowish precipitate was observed on day 7.

### Example 67-148

HA-Pt (10 mg/mL) was mixed with NaCl (5 mg/mL) and mannitol (290 mOsm) at pH 7.4 and incubated at 25° C. Yellowish precipitate was observed on day 30.

### Example 67-149

HA-Pt (10 mg/mL) was mixed with NaCl (5.5 mg/mL) and mannitol (290 mOsm) at pH 7.4 and incubated at 25° C. Yellowish precipitate was observed on day 6.

### Example 67-150

HA-Pt (10 mg/mL) was mixed with NaCl (6 mg/mL) and mannitol (290 mOsm) at pH 7.4 and incubated at 25° C. Yellowish precipitate was observed on day 6.

### Example 67-151

HA-Pt (10 mg/mL) was mixed with NaCl (6.5 mg/mL) and mannitol (290 mOsm) at pH 7.4 and incubated at 25° C. Yellowish precipitate was observed on day 5.

### Example 67-152

HA-Pt (10 mg/mL) was mixed with NaCl (7 mg/mL) and mannitol (290 mOsm) at pH 7.4 and incubated at 25° C. Yellowish precipitate was observed on day 4.

### Example 67-153

HA-Pt (10 mg/mL) was mixed with NaCl (7.5 mg/mL) and mannitol (290 mOsm) at pH 7.4 and incubated at 25° C. Yellowish precipitate was observed on day 4.

### Example 67-154

HA-Pt (10 mg/mL) was mixed with NaCl (8 mg/mL) and mannitol (290 mOsm) at pH 7.4 and incubated at 25° C. Yellowish precipitate was observed on day 3.

### Example 67-155

HA-Pt (10 mg/mL) was mixed with NaCl (8.5 mg/mL) and mannitol (290 mOsm) at pH 7.4 and incubated at 25° C. Yellowish precipitate was observed on day 3.

### Example 67-156

HA-Pt (10 mg/mL) was mixed with NaCl (9 mg/mL) and mannitol (290 mOsm) at pH 7.4 and incubated at 25° C. Yellowish precipitate was observed on day 3.

### Example 68

### Lyophilization Using Trehalose and Mannitol Formulation

The HA-Pt (29-39 kDa) was lyophilized using a VirTis Advantage benchtop freeze dryer (SP Scientific, Warminster, Pa.). Prior to lyophilizing, 2.5% wt each trehalose or mannitol was added to the HA-Pt as a cryoprotectant. The pH was adjusted to 6.8±0.1 or 7.4±0.1 using 1 N NaOH. One hundred milliliters of the HA-Pt was aliquoted into twenty 5-mL glass vials and was frozen in a −80° C. freezer for 4 hours. The vials were transferred to the freeze dryer and kept at −40° C. for 1.5 hours. The condenser point was set at −70° C. The vacuum setpoint was 100 mTorr. The parameters for primary drying were 30 hours at −35° C. and 10 mTorr. The parameters for secondary drying were 5 hours at 2° C. and 10 mTorr, followed by another 5 hours at 8° C. and 10 mTorr. Once the drying was complete, vials containing the lyophilized HA-Pt were stored at −20° C. in the dark. The concentration of the lyophilized formulation was 2-8 mg/mL on cisplatin basis. The lyophilized formulation is a white or light yellow colored cake. The lyophilized HA-Pt may be rehydrated using either water or 0.9% wt saline.

### Example 69

### Lyophilization Using Trehalose and Sodium Chloride Formulation

The HA-Pt (29-39 kDa) was lyophilized using a VirTis Advantage benchtop freeze dryer (SP Scientific, Warminster, Pa.). Prior to lyophilizing, 10% wt trehalose was added to the HA-Pt as a cryoprotectant and 0.9% wt NaCl was also added to prevent the formation of free platinums. The drug solution was filtered through a 0.22 um PES filter. Two to five milliliters of the HA-Pt was aliquoted into 10-mL glass vials and was frozen in a -80° C. freezer for 16 hours. The condenser point was set at −70° C. The vacuum setpoint was 100 mTorr. The parameters for primary drying were 4320 min at −40° C. and 100 mTorr. The parameters for secondary drying were 300 mins at 2° C. and 100 mTorr, followed by another 300 mins at 8° C. and 100 mTorr. Once the drying was complete, vials containing the drug will be degassed at 0-2° C., <100 mtorr, for 30-60 min. Subsequently, vials containing the lyophilized HA-Pt were stored at −20° C. in the dark. The concentration of the lyophilized formulation was 2-8 mg/mL on cisplatin basis. The lyophilized formulation is a white or light yellow colored cake.

### Example 70

### Rehydration of Lyophilized HA-Pt

The lyophilized HA-Pt may be rehydrated using either water or high molecular weight HA (e.g. 0.8, 1.3 and 2 MDa). It takes less than 30 seconds t rehydrate HA-Pt using water. It takes approximately 10 min to rehydrate HA-Pt using diluents containing high molecular weight HA. The concentration of high molecular weight HA was 1% wt for 0.8 MDa, 1% wt for 1.3 MDa, and 1 and 0.5% wt for 2 MDa.

### Example 71

### Mouse MTD Study of Lyophilized HA-Pt

Maximum Tolerated Dose (MTD) study was conducted in healthy Blab/C mice at 2 and 4 mg/kg using either HA-Pt alone or HA-Pt in the presence of high molecular weight HA (HMW). At the lower dose, 100% of animals survived in either HA-Pt alone or HA-Pt+HMW group. At the higher dose, 100% of animals deceased in the HA-Pt alone arm, whereas 100% of animals survived in the HA-Pt+HWM arm though weight loss was observed for the majority of the animals. 17 days post chemo, the number of mice that are still below prestudy weight are 1/6 for 2 MDa groups, 2/6 for 1.3 MDa group and 3/6 for 0.8 MDa group. Based on the result, HA-Pt+HWM is more tolerable than HA-Pt alone in mice. Higher molecular weight HA may be protective against toxicity than medium molecular weight HA.

### Example 72

### Rabbit Toxicity Study of Lyophilized HA-Pt

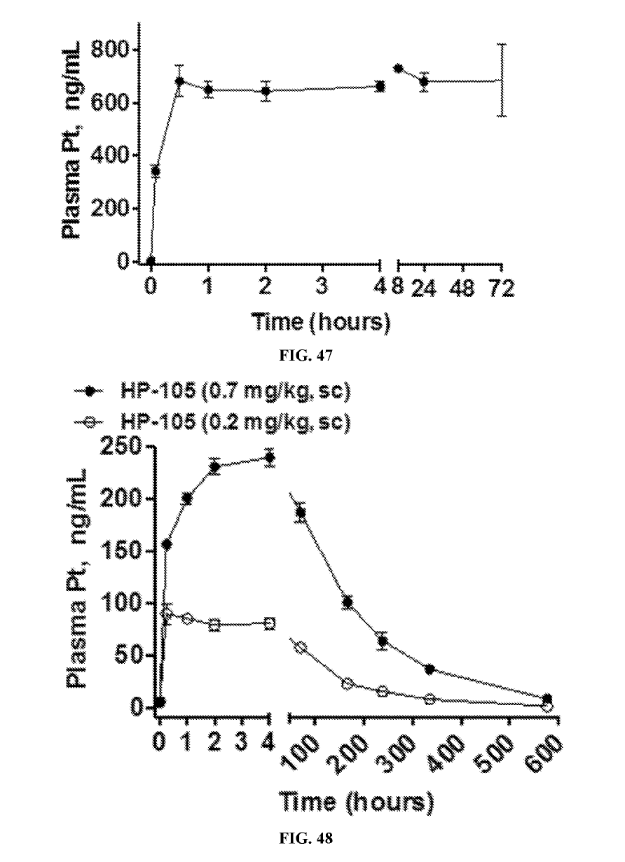

Rabbit toxicity study was conducted in healthy rabbits using either HA-Pt alone or HA-Pt+HWM at 0.2, 0.7, 1 and 2 mg/kg. Blood samples were collected for PK, Complete Blood Count (CBC) and Chemistry. The highest dose, 2 mg/kg HA-Pt alone, was found to be lethal. 4/4 died within 5 days. Blood work indicated kidney and liver failure in all rabbits. PK did not demonstrate an elimination phase possibly due to kidney failure. PK result is shown in FIG. 47 for rabbits dosed with 2 mg/kg HA-Pt alone.

### Example 73

### Rabbit Toxicity Study of Lyophilized HA-Pt

A second toxicity study was conducted at two lower doses that were 0.2 and 0.7 mg/kg. 4/4 rabbits survived. The doses were found to be insufficient to induce changes in blood work. 2/2 in 0.2 mg/kg group had no changes in blood work. 1/2 in 0.7 mg/kg group had very mild change and 1/2 had no changes. PK result showed linear pharmacokinetics between the two lower doses. See FIG. 48 for PK of low dose HA-Pt.

### Example 74

### Rabbit Toxicity Study of Lyophilized HA-Pt

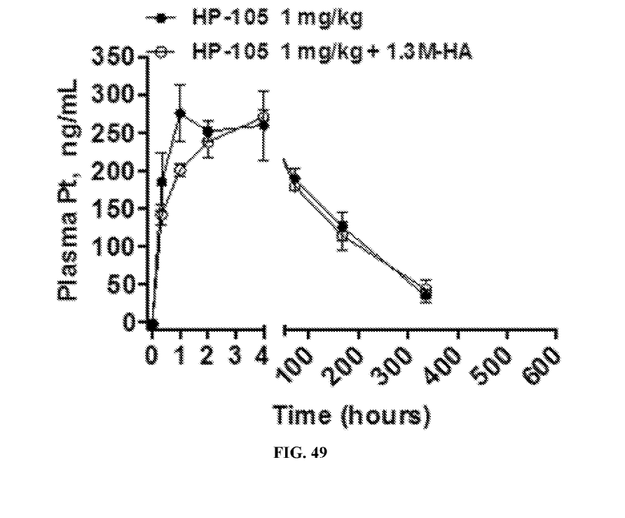

A third toxicity and PK study was performed at a medium dose, 1 mg/kg, using either HA-Pt alone or HA-Pt+HMW. Neither HA-Pt nor HA-Pt+HMW caused animal death in 10 days. The dose was sufficient to induce changes of liver enzymes and platelet counts. See FIG. 49 for PK. HA-Pt alone caused transient thrombocytopenia in both rabbits. The platelet counts of both rabbits returned to prestudy values within 10 days. HMW did not cause thrombocytopenia in either rabbits over 10 days. In addition, HA-Pt alone caused mild elevation of liver enzymes in one rabbit and severe elevation of liver enzymes in the other rabbit. Both rabbits' liver enzymes normalized by the end of 10 days. In comparison, HWM only caused mild elevation of liver enzyme in one rabbit. The other rabbit had no change in liver enzymes. Result was based on N=2 per drug. In summary, HA-Pt+HWM treatment demonstrated delayed Tmax compared to HA-Pt alone. Biphasic PK indicated sustained release over 10 days. HA-Pt+HMW did not result in thrombocytopenia in rabbits whereas HA-Pt alone did. HA-Pt+HMW resulted in milder elevations in liver enzymes compared to HA-Pt alone.

### Example 75

### Dog Study of Lyophilized HA-Pt

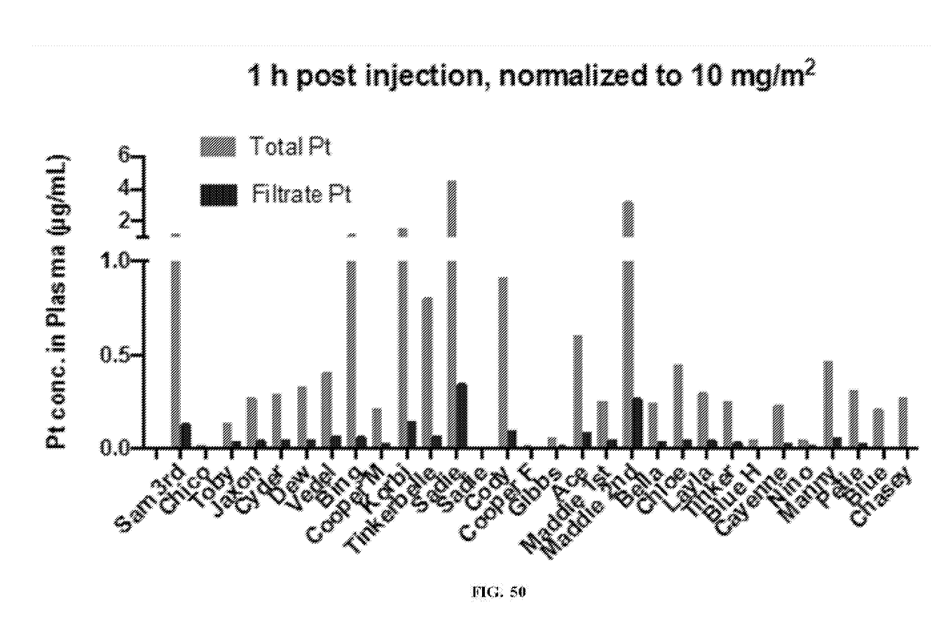

Total and free Pt levels were determined in study dogs at 1-hr post chemo. See FIG. 50. The last four dogs received HA-Pt+HMW, and all other dogs received HA-Pt alone. Pt levels in dogs treated with HA-Pt alone were very variable. In comparison, HA-Pt+HMW resulted in a narrow range of Pt blood levels though 4 dogs had different cancers of variable sizes and vascularity. This interesting observation will be confirmed after more dogs are treated with HA-Pt+HMW. In summary, PK of HA-Pt+HMW may be less variable than HA-Pt alone in dogs, which may translate into improved overall safety as high peak plasma levels of Pt is associated with toxicity.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

We claim:

1. A composition comprising:
a drug conjugate, the drug conjugate comprising:
hyaluronan;
a chelated platinum; and
a linker joining the hyaluronan and the platinum,
wherein the linker comprises an amine, amide or carboxyl group capable of chelating the platinum.

2. The composition of claim 1, wherein the linker comprises at least two groups selected from the group consisting of amine, amide and carboxyl, wherein each of the two groups may be the same or different.

3. The composition of claim 1, wherein the linker comprises a modified amino acid capable of chelating the platinum.

4. The composition of claim 3, wherein the linker comprises one or two modified amino acids.

5. The composition of claim 4, wherein the linker comprises a derivative of lysine.

6. The composition of claim 5, wherein the derivative of lysine is N-acetyl lysine.

7. The composition of claim 1, wherein the linker comprises a ligand selected from the group consisting of diglycine, dicarboxylato, maloyl, and diamino.

8. The composition of claim 1, wherein the linker comprises HOOC—X—NH—Y,
wherein X is selected from the group consisting of CH₂, CH₂Z, and CHZCH₂,
wherein Z is an aliphatic group, and
wherein Y is a moiety selected from the group consisting of acetyl, ethyl and an aliphatic chain.

9. The composition of any of claims 1-8, wherein the linker further comprises a hydrazide.

10. The composition of any of claims 1-9, wherein the platinum is in the II oxidative state.

11. The composition of any of claims 1-10, wherein the linker and the platinum form a ring selected from the group consisting of 5 member rings and 6 member rings.

12. The composition of claim 11, wherein the linker and the platinum form a 6 member ring.

13. The composition of any of claims 1-12, wherein the platinum forms part of a drug selected from the group consisting of cisplatin, carboplatin and oxaliplatin.

14. The composition of any of claims 1-13, wherein the hyaluronan has a molecular weight from 6 to 300 kDa.

15. The composition of claim 14, wherein the composition further comprises in addition to the drug conjugate, a second hyaluronan having a molecular weight greater than 600 kDa.

16. The composition of any of claims 1-15, wherein the composition further comprises an excipient selected from the group consisting of a sugar, a monovalent or polyvalent cation, and combinations thereof.

17. The composition of claim 16, wherein the sugar is mannitol, sucrose, or trehalose.

18. The composition of claim 16, wherein the cation is a calcium salt or sodium salt.

19. A method for treating and/or inhibiting cancer, the method comprising:
administering the composition of any of claims 1-18 to a patient in need thereof.

20. A process for lyophilizing a hyaluronan-drug conjugate comprising:
forming a lyophilization formulation comprising:
a hyaluronan-drug conjugate; and
an excipient selected from the group consisting of a salt having a concentration from 0.01 to 0.9 wt %, a sugar having a concentration between 1 and 20 wt % and combinations thereof; and
lyophilizing the conjugate.

21. The process of claim 20, wherein the lyophilizing step comprises a freezing step following by a multistep drying process.

22. The process of claim 20 or 21, wherein the salt is selected from the group consisting of calcium and sodium salts and the sugar is selected from the group consisting of mannitol, sucrose, or trehalose.

23. The process of any of claims 20-22, further comprising rehydrating the conjugate with a diluent comprising a hyaluronan with a molecular weight greater than 600 kDa.

24. The process of any of claims 20-23, wherein the hyaluronan-drug conjugate is a hyaluronan-platinum conjugate.

25. The process of claim 24, wherein the hyaluronan-platinum conjugate comprises:
a hyaluronan;
a chelated platinum; and
a linker joining the hyaluronan and the platinum,
wherein the linker comprises an amine, amide or carboxyl group capable of chelating the platinum.
